(19) United States
(12) Patent Application Publication
(10) Pub. No.: US 2026/0008865 A1
(43) Pub. Date: Jan. 8, 2026

(54) ANTIBODIES, COMPOSITIONS, AND METHODS OF TREATMENT

(71) Applicant: Tetherex Pharmaceuticals Corporation, Oklahoma City, OK (US)

(72) Inventor: Russell ROTHER, Oklahoma City, OK (US)

(21) Appl. No.: 19/111,279

(22) PCT Filed: Sep. 13, 2023
(86) PCT No.: PCT/IB2023/059093

§ 371 Date: Mar. 12, 2025

Related U.S. Application Data
(60) Provisional application No. 63/406,069, filed on Sep. 13, 2022.
(60) Provisional application No. 63/406,545, filed on Sep. 14, 2022.
(60) Provisional application No. 63/452,896, filed on Mar. 17, 2023.

(51) Int. Cl. C07K 16/28; A61K 39/00; A61P 11/00; A61P 37/02

(52) U.S. Cl. CPC C07K 16/2896; A61P 11/00; A61P 37/02; A61K 2039/505; C07K 2317/24; C07K 2317/565; C07K 2317/76; C07K 2317/92

(57) ABSTRACT

The invention provides anti-PSGL-1 antibodies, and their therapeutic uses for the prevention and or treatment of dis-cases. In particular, the diseases to be prevented and or treated may be inflammatory diseases (particularly involving accumulation of eosinophils and/or neutrophils). Treatments may be effective in respect of asthma or COPD. The antibodies may be humanised, and may be defined with reference to sequences set out in the specification. The antibodies may have high affinities for PSGL-1.

### FIELD OF INVENTION

The present invention relates to anti-human PSGL-1 antibodies. The invention also relates to a method of treatment of a variety of diseases and conditions, by decreasing inflammation.

### BACKGROUND

P-selectin glycoprotein ligand-1 (PSGL-1) is a component of the innate immune system and is found on leukocytes and endothelial cells. PSGL-1 binds to members of the selectin family of adhesion molecules including P-, E-, and L-selectin, but binds with highest affinity to P-selectin. PSGL-1 also binds key chemokines known to induce chemotaxis of cells into areas of inflammation.

PSGL-1 facilitates binding between leukocytes, platelets and the endothelium at sites of hypoxia, endothelial cell damage, and infection. Normally a beneficial process, when activation of these cells occurs unnecessarily or is excessive, PSGL-1-mediated cell-cell interactions can cause a cascade of events culminating in inflammatory cell extravasation into tissues such as the lung parenchyma and bronchoalveolar space resulting in pathologic inflammation.

Asthma and chronic obstructive pulmonary disease (COPD) are common pulmonary inflammatory diseases characterized by excessive leukocyte infiltration into the lung parenchyma and bronchoalveolar space, resulting in tissue damage. Asthma is a common chronic inflammatory disease of the lungs characterized by episodes of wheezing, breathlessness, chest tightness, and cough, and the disease is often associated with eosinophil-rich airway inflammation. Airway eosinophilia is associated with asthma exacerbations and has been shown to play a role in airway remodelling. COPD represents one of the leading causes of death worldwide, and there are few effective therapies. Foreign particulate matter such as that produced from cigarette smoke leads to the recruitment of inflammatory cells to the bronchial wall and lumen resulting in progressive loss of lung function. Accumulation of neutrophils at sites of inflammation occurring in COPD may contribute to the pathophysiology of this disease, for example by secreting proteases that cause tissue destruction, or releasing mediators that further increase or prolong the inflammatory response.

Current therapies for moderate to severe asthma include inhaled corticosteroids, inhaled long- or short acting beta-agonists, inhaled long- or short-acting anticholinergics, leukotriene modifiers, cromolyn sodium, theophylline, or oral corticosteroids. Five biologics are also currently approved to treat severe asthma, including reslizumab (for eosinophilic asthma; anti-IL-5), mepolizumab (for eosinophilic asthma; anti-IL-5), omalizumab (for allergic asthma; anti-IgE), benralizumab (for eosinophilic asthma; anti-IL-5 receptor), and tezepelumab (for severe asthma; thymic stromal lymphopoietin).

Current therapies for COPD include short acting and long-acting 32 agonists and inhaled corticosteroid combination therapy, theophylline and antibiotics. These therapies are effective in some individuals, but exacerbations still occur and there are currently no disease-modifying therapies. Although neutrophil-mediated inflammation in COPD is thought to play a key role in the pathophysiology of the disease, there is still a lack of effective anti-inflammatory therapies. The anti-inflammatory phosphodiesterase-4 PDE4 inhibitor roflumilast was approved by the FDA in 2011 for patients with COPD. PDE4 inhibition has shown some promise but it appears to be effective in only a subset of patients and is associated with nausea, diarrhea, headaches and weight loss.

There is still a strong unmet clinical need for therapies in pulmonary diseases, such as asthma or COPD, and in particular for pulmonary diseases characterized by airway eosinophilia and/or neutrophilia.

Sickle cell disease (SCD) is a genetic blood disorder characterized by the presence of abnormal haemoglobin known as haemoglobin S (HbS). This abnormal haemoglobin causes red blood cells to take on a characteristic sickle or crescent shape. This in turn leads to pathological features including vaso-occlusion (where sickle-shaped red blood cells clump together and block small blood vessels), and chronic inflammation. These processes may lead to symptoms of SCD such as pain crises and tissue damage.

There is a strong unmet clinical need for therapies in SCD, and its symptoms such as pain crisis and chronic inflammation.

Indeed, there remains an unmet need for therapies for inflammatory diseases or disorders.

### SUMMARY OF THE INVENTION

According to a first aspect of the invention, there is provided a humanised anti-PSGL-1 antibody, or antigen binding fragment thereof, comprising at least one CDR of the antibody heavy chain set out in SEQ ID NO: 1 and at least one CDR of the antibody light chain set out in SEQ ID NO: 2.

In a suitable embodiment a humanised anti-PSGL-1 antibody, or antigen binding fragment thereof, in accordance with the first aspect of the invention comprises all three CDRs of the antibody heavy chain set out in SEQ ID NO: 1 and all three CDRs of the antibody light chain set out in SEQ ID NO: 2.

In a second aspect, the invention provides an anti-PSGL-1 antibody, or antigen binding fragment thereof:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2.

According to a third aspect of the invention, there is provided a pharmaceutical composition comprising a humanised anti-PSGL-1 antibody, or antigen binding fragment thereof, comprising at least one CDR of the antibody heavy chain set out in SEQ ID NO: 1 and at least one CDR of the antibody light chain set out in SEQ ID NO: 2 and a pharmaceutically acceptable excipient.

In a suitable embodiment the humanised anti-PSGL-1 antibody, or antigen binding fragment thereof, present in a pharmaceutical composition in accordance with the third aspect of the invention comprises all three CDRs of the antibody heavy chain set out in SEQ ID NO: 1 and all three CDRs of the antibody light chain set out in SEQ ID NO: 2.

According to a fourth aspect of the invention, there is provided a polynucleotide sequence encoding an immunoglobulin chain of a humanised anti-PSGL-1 antibody, or an antigen binding fragment thereof, comprising at least one CDR of the antibody heavy chain set out in SEQ ID NO: 1 and at least one CDR of the antibody light chain set out in SEQ ID NO: 2.

According to a fifth aspect of the invention, there is provided a polynucleotide sequence encoding a humanised anti-PSGL-1 antibody, or antigen binding fragment thereof, comprising an immunoglobulin chain comprising at least one CDR of the antibody heavy chain set out in SEQ ID NO: 1 and an immunoglobulin chain comprising at least one CDR of the antibody light chain set out in SEQ ID NO: 2.

In a suitable embodiment a polynucleotide sequence in accordance with the fourth or fifth aspect of the invention encodes an immunoglobulin chain that comprises all three CDRs of the antibody heavy chain set out in SEQ ID NO: 1. In a suitable embodiment a polynucleotide sequence in accordance with the fourth or fifth aspect of the invention encodes an immunoglobulin chain that comprises all three CDRs of the antibody light chain set out in SEQ ID NO: 2. In a suitable embodiment a polynucleotide sequence in accordance with the fourth aspect of the invention encodes an immunoglobulin chain that comprises all three CDRs of the antibody heavy chain set out in SEQ ID NO: 1 and all three CDRs of the antibody light chain set out in SEQ ID NO: 2. In a suitable embodiment a polynucleotide sequence in accordance with the fifth aspect of the invention encodes an immunoglobulin chain that comprises all three CDRs of the antibody heavy chain set out in SEQ ID NO: 1 and an immunoglobulin chain that comprises all three CDRs of the antibody light chain set out in SEQ ID NO: 2.

According to a sixth aspect of the invention, there is provided a cell comprising a polynucleotide sequence encoding an immunoglobulin chain of a humanised anti-PSGL-1 antibody, or antigen binding fragment thereof, comprising at least one CDR of the antibody heavy chain set out in SEQ ID NO: 1 and at least one CDR of the antibody light chain set out in SEQ ID NO: 2.

According to a seventh aspect of the invention, there is provided a cell comprising a polynucleotide sequence encoding a humanised anti-PSGL-1 antibody, or antigen binding fragment thereof, comprising an immunoglobulin chain comprising at least one CDR of the antibody heavy chain set out in SEQ ID NO: 1 and an immunoglobulin chain comprising at least one CDR of the antibody light chain set out in SEQ ID NO: 2.

In a suitable embodiment a polynucleotide sequence present in a cell in accordance with the sixth or seventh aspect of the invention encodes an immunoglobulin chain that comprises all three CDRs of the antibody heavy chain set out in SEQ ID NO: 1. In a suitable embodiment a polynucleotide sequence present in a cell in accordance with the sixth or seventh aspect of the invention encodes an immunoglobulin chain that comprises all three CDRs of the antibody light chain set out in SEQ ID NO: 2. In a suitable embodiment a polynucleotide sequence present in a cell in accordance with the sixth aspect of the invention encodes an immunoglobulin chain that comprises all three CDRs of the antibody heavy chain set out in SEQ ID NO: 1 and all three CDRs of the antibody light chain set out in SEQ ID NO: 2. In a suitable embodiment a polynucleotide sequence present in a cell in accordance with the seventh aspect of the invention encodes an immunoglobulin chain that comprises all three CDRs of the antibody heavy chain set out in SEQ ID NO: 1 and an immunoglobulin chain that comprises all three CDRs of the antibody light chain set out in SEQ ID NO: 2.

According to a eighth aspect of the invention, there is provided a method of manufacturing a humanised anti-PSGL-1 antibody, or an antigen binding fragment thereof, comprising at least one CDR of the antibody heavy chain set out in SEQ ID NO: 1 and at least one CDR of the antibody light chain set out in SEQ ID NO: 2, the method comprising:
expressing a first polynucleotide sequence encoding an immunoglobulin chain of a humanised anti-PSGL-1 antibody, or an antigen binding fragment thereof, comprising at least one CDR of the antibody heavy chain set out in SEQ ID NO: 1; and
expressing a second polynucleotide sequence encoding an immunoglobulin chain of a humanised anti-PSGL-1 antibody, or an antigen binding fragment thereof, comprising at least one CDR of the antibody light chain set out in SEQ ID NO: 2.

In a suitable embodiment, a first polynucleotide sequence expressed in a method in accordance with the eighth aspect of the invention encodes an immunoglobulin chain that comprises all three CDRs of the antibody heavy chain set out in SEQ ID NO: 1. In a suitable embodiment a second polynucleotide sequence expressed in a method in accordance with the eighth aspect of the invention encodes an immunoglobulin chain that comprises all three CDRs of the antibody light chain set out in SEQ ID NO: 2. In a suitable embodiment, a first polynucleotide sequence expressed in a method in accordance with the eighth aspect of the invention encodes an immunoglobulin chain that comprises all three CDRs of the antibody heavy chain set out in SEQ ID NO: 1, and a second polynucleotide expressed in a method in accordance with the eighth aspect of the invention encodes an immunoglobulin chain that comprises all three CDRs of the antibody light chain set out in SEQ ID NO: 2.

According to a ninth aspect of the invention, there is provided a method of preventing or treating a disease or condition in a subject in need thereof, the method comprising providing a therapeutically effective amount of an anti-PSGL-1 antibody, or antigen binding fragment thereof, to the subject, wherein the antibody comprises at least one CDR of the antibody heavy chain set out in SEQ ID NO: 1 and at least one CDR of the antibody light chain set out in SEQ ID NO: 2. The antibody may be an anti-PSGL-1 antibody, or antigen binding fragment thereof, in accordance with any embodiment of the first aspect of the invention, or an anti-PSGL-1 antibody or antigen binding fragment thereof in accordance with the second aspect of the invention. In a suitable embodiment, the antibody, or antigen binding fragment thereof, comprises all three CDRs of the antibody heavy chain set out in SEQ ID NO: 1 and all three CDRs of the antibody light chain set out in SEQ ID NO: 2.

According to an tenth aspect of the invention, there is provided an anti-PSGL-1 antibody, or antigen binding fragment thereof, comprising at least one CDR of the antibody heavy chain set out in SEQ ID NO: 1 and at least one CDR of the antibody light chain set out in SEQ ID NO: 2, for use as a medicament. The antibody may be an anti-PSGL-1 antibody, or antigen binding fragment thereof, in accordance with any embodiment of the first aspect of the invention, or an anti-PSGL-1 antibody, or antigen binding fragment thereof, in accordance with the second aspect of the invention. In a suitable embodiment, the antibody, or antigen binding fragment thereof, comprises all three CDRs of the antibody heavy chain set out in SEQ ID NO: 1 and all three CDRs of the antibody light chain set out in SEQ ID NO: 2.

Antibodies of the invention in accordance with the first or second aspect of the invention, or antigen binding fragments thereof, are suitable for use in the methods of treatment of the invention or in the medical uses of the invention of the ninth or tenth aspects of the invention. They may also be incorporated in the pharmaceutical compositions of the third aspect of the invention.

Methods of treatment in accordance with the ninth aspect of the invention, or antibodies, or antigen binding fragments thereof, for use in accordance with the tenth aspect of the invention, may be for use in the prevention or treatment of diseases or conditions associated with binding of PSGL-1 to its ligands.

Antibodies, or antigen binding fragments thereof, for use in accordance with the tenth aspect of the invention may be used in the form of a pharmaceutical composition comprising the antibody, or antigen binding fragment thereof, and a pharmaceutically acceptable excipient, such as a composition of the third aspect of the invention.

In an eleventh aspect of the invention there is provided a polynucleotide sequence encoding the heavy chain variable sequence set out in SEQ ID NO: 26.

In a twelfth aspect of the invention there is provided a polynucleotide sequence encoding the light chain variable sequence set out in SEQ ID NO: 29.

In a thirteenth aspect of the invention, there is provided a cell comprising a polynucleotide sequence encoding the heavy chain sequence set out in SEQ ID NO: 1 and a polynucleotide sequence encoding the light chain sequence set out in SEQ ID NO: 2.

For the purposes of the present disclosure, a therapeutically effective amount of an antibody, or an antigen binding fragment thereof, or of a pharmaceutical composition, may be taken as referring to a quantity or dose of the antibody or fragment, or of a pharmaceutical composition comprising the antibody or fragment, that is sufficient to produce a desired therapeutic or beneficial effect in a patient or individual. A therapeutically effective amount may be an amount sufficient to reduce or delay the onset of a disease or condition, or an amount sufficient to reduce or delay the onset of one or more symptoms of a disease or condition. A therapeutically effective amount may be an amount sufficient to provide at least partial relief in respect of a disease or condition, or an amount sufficient to provide at least partial relief in respect of one or more symptoms of a disease or condition. A therapeutically effective amount may be an amount sufficient to entirely prevent the onset of a disease or condition, or an amount sufficient to entirely prevent the onset of one or more symptoms of a disease or condition. A therapeutically effective amount may be an amount sufficient to entirely alleviate a disease or condition, or an amount sufficient to entirely alleviate one or more symptoms of a disease or condition. Other specific considerations regarding therapeutically effective amounts antibodies, antibody fragments, or pharmaceutical compositions, taking into account particular therapeutic outcomes of interest, are set out elsewhere in this specification.

### BRIEF DESCRIPTION OF THE FIGS

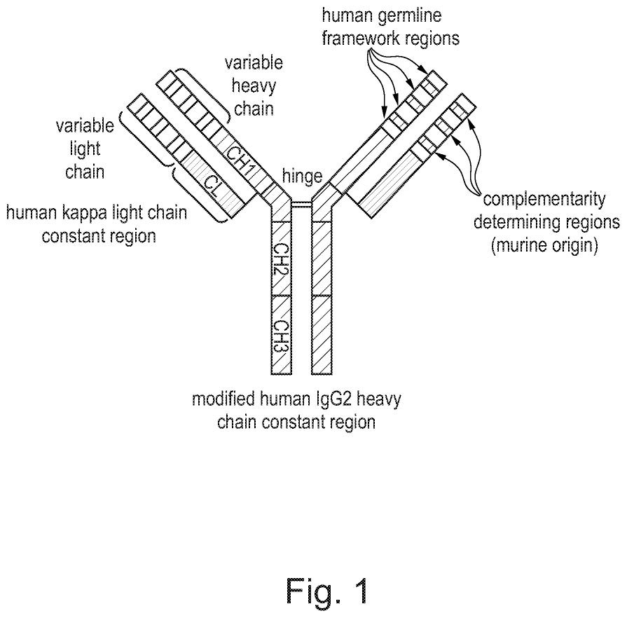

FIG. 1 shows a schematic drawing of SelK2 antibody and demonstrates the arrangement and linkage of the antibody domains.

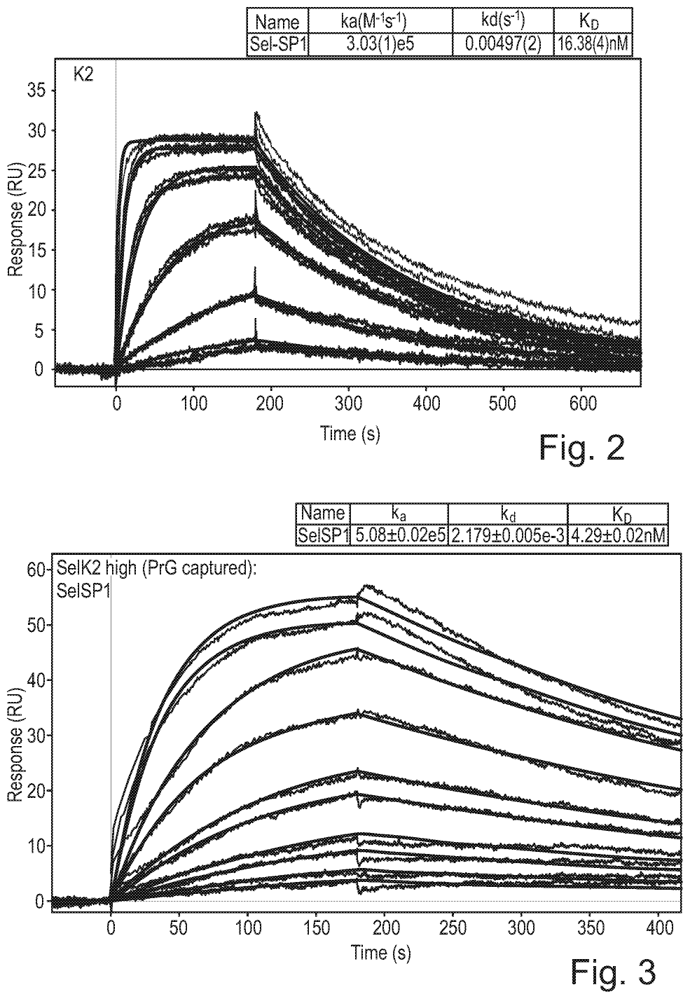

FIG. 2 shows the results of a kinetic assay of SelSP1 binding to captured parental antibody.

FIG. 3 shows the results of a kinetic assay of SelSP1 binding to captured SelK2 (high density surface).

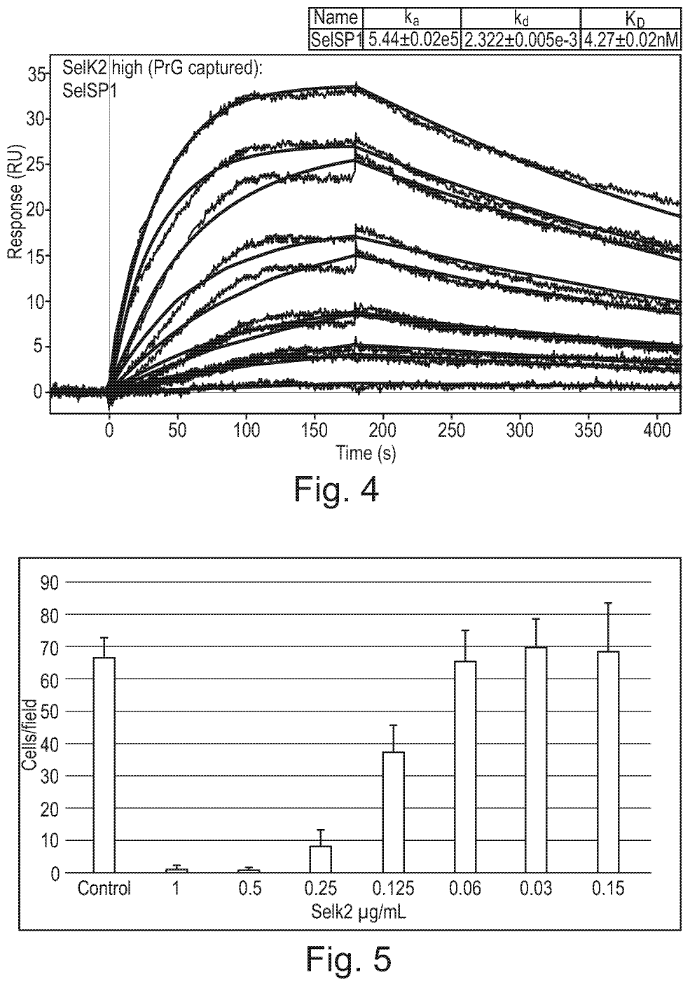

FIG. 4 shows the results of a kinetic assay of SelSP1 binding to captured SelK2 (low density surface).

FIG. 5 shows the results of a study demonstrating the ability of SelK2 to inhibit neutrophil rolling.

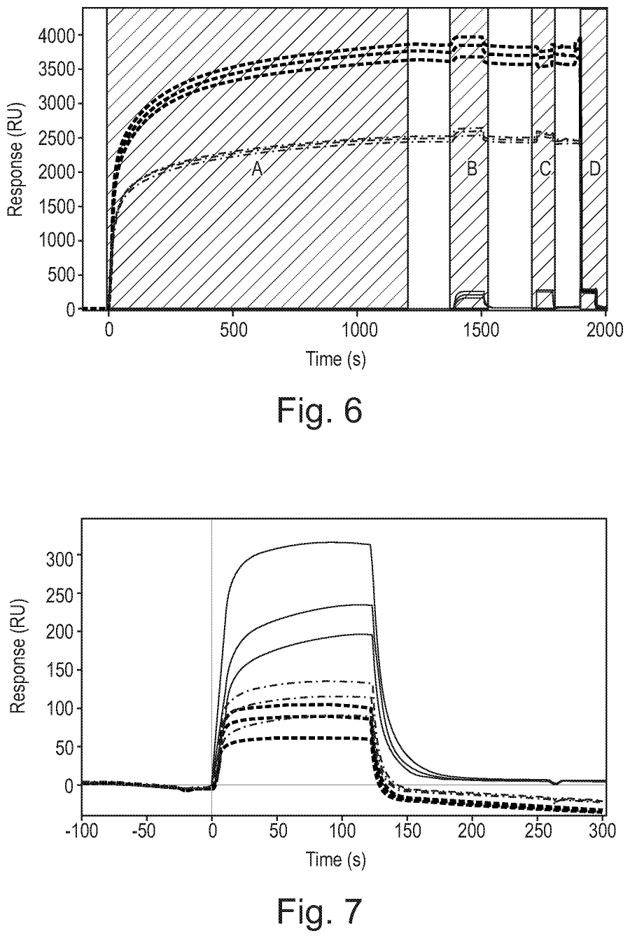

FIG. 6 illustrates parental antibody and SelK2 antibody of the invention inhibiting chemokine CCL27 binding and interaction with PSGL-1. CCL27 complete assay cycle. Injection ‘A’ is surface blocking, injection ‘B’ is CCL27, injection ‘C’ is SDS, and injection ‘D’ is NaOH.

FIG. 7 illustrates the blocking of parental and SelK2 antibody zoomed into injection ‘B’ and re-normalized before injection. CCL27 (990 nM) injection. Solid line curves represent buffer blocked surface, dot-dash line curves represent K2 blocked surface and dashed line curves represent SelK2 blocked surface.

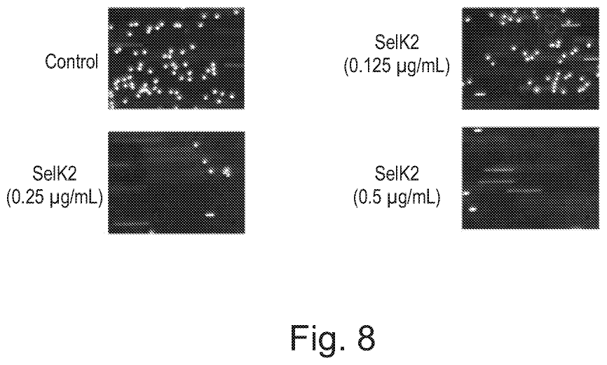

FIG. 8 illustrates SelK2 inhibition of human neutrophil binding to P-selectin under flow.

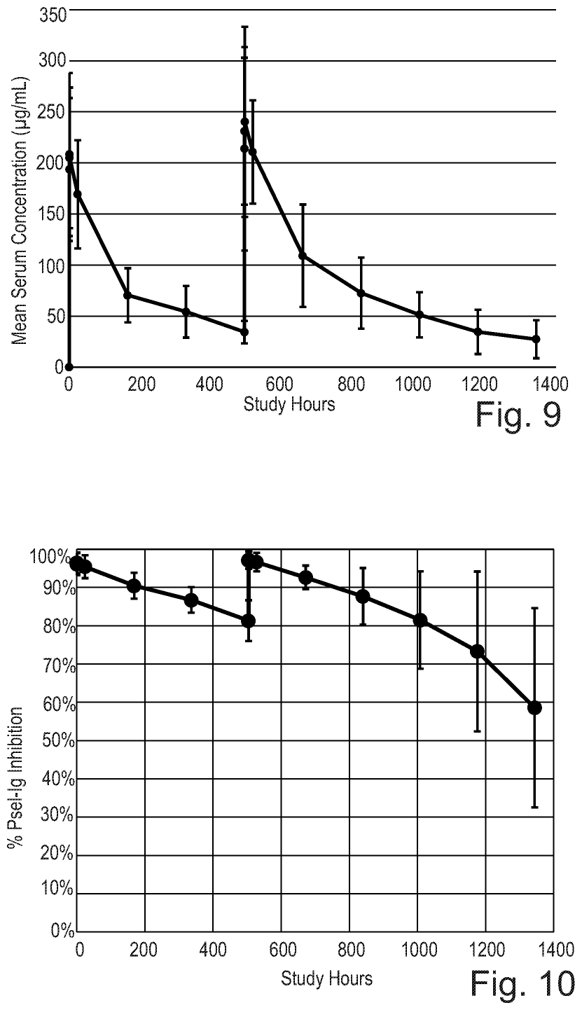

FIG. 9 shows the change in mean serum concentration of SelK2 over time (PK) after administration of two 7.5 mg/kg doses 21 days apart.

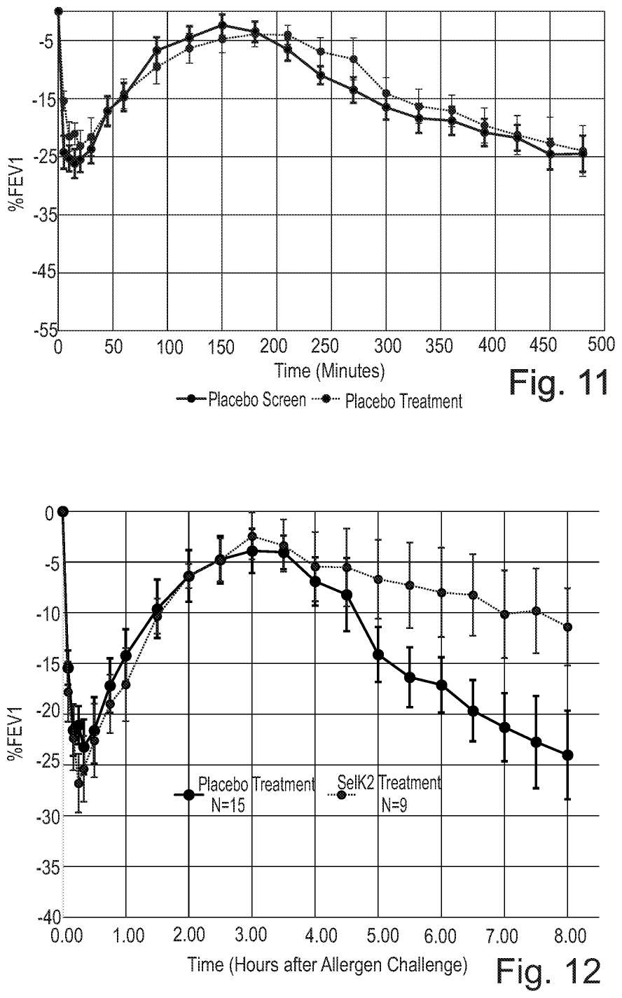

FIG. 10 illustrates the change in mean % Psel-Ig Inhibition in response to SelK2 serum concentration over time (PD) after administration of two 7.5 mg/kg doses 21 days apart.

FIG. 11 shows the % Fall in FEV1 in placebo subjects during screening versus during treatment following allergen challenge (Mean±SE).

FIG. 12 shows the % Fall in FEV1 at Day 36 (after 5 weeks of treatment) following allergen challenge in asthmatic subjects receiving placebo or SelK2 treatment in accordance with the invention (Mean±SE).

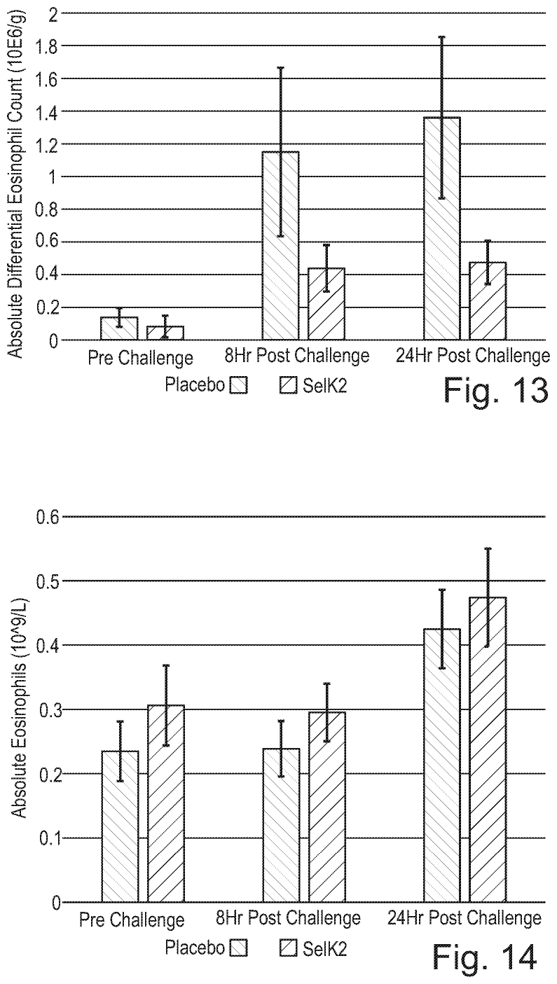

FIG. 13 shows mean (±SE) absolute differential eosinophil count (10E6/g) in sputum of asthmatic patients treated with placebo or SelK2 following allergen challenge.

FIG. 14 shows mean (±SE) absolute eosinophils count (10E9/L) in blood of asthmatic patients treated with placebo or SelK2 following allergen challenge.

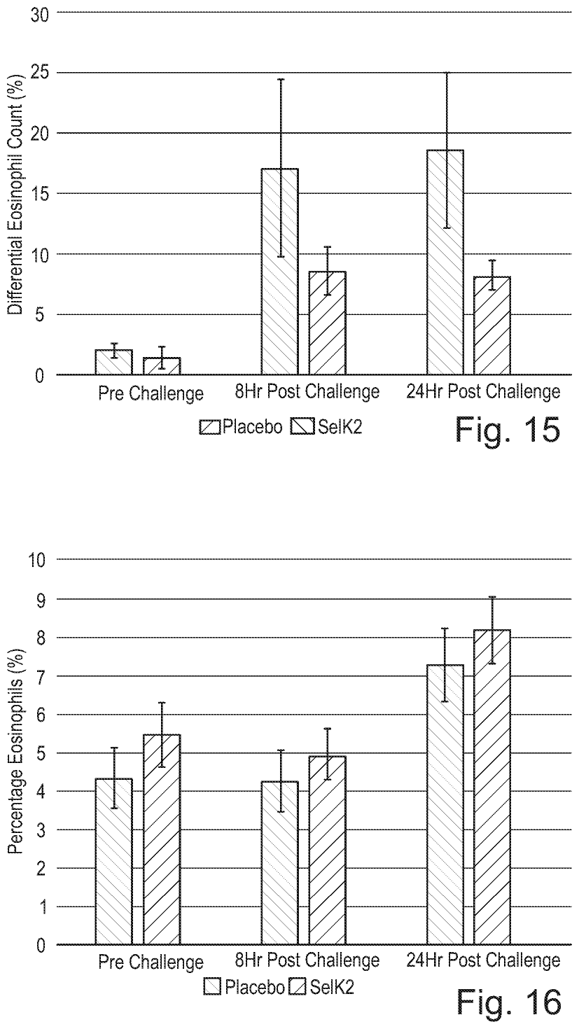

FIG. 15 illustrates the mean (±SE) differential eosinophil count (%) in sputum of asthmatic patients treated with placebo or SelK2 following allergen challenge.

FIG. 16 illustrates the mean (±SE) differential eosinophil count (%) in blood of asthmatic patients treated with placebo or SelK2 following allergen challenge.

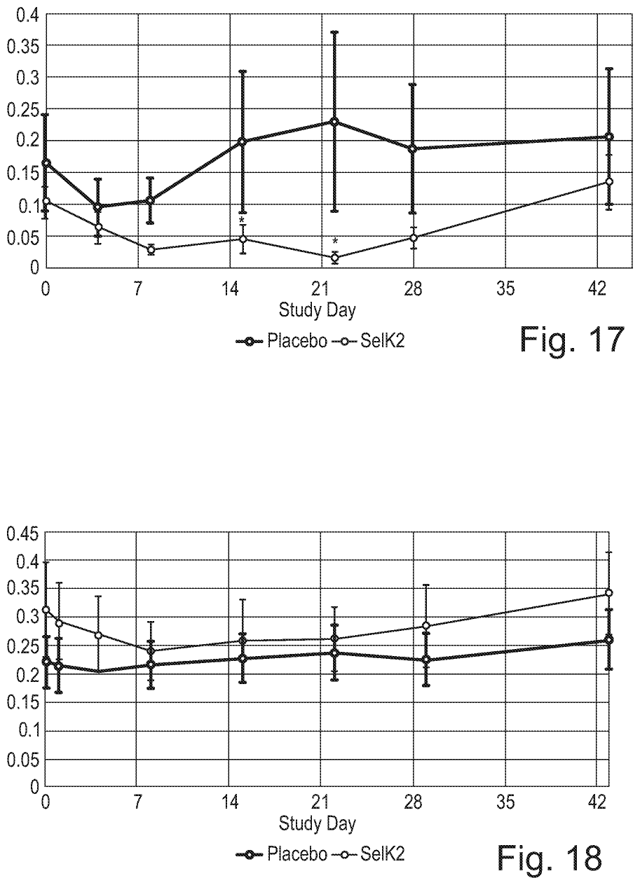

FIG. 17 shows mean (±SE) absolute differential eosinophil count (10E6/g) in sputum of COPD patients treated with placebo or SelK2. Day 15, p=0.0494, Day 22, p=0.0181.

FIG. 18 shows mean (±SE) absolute differential eosinophil count (10E6/L) in blood of COPD patients treated with placebo or SelK2.

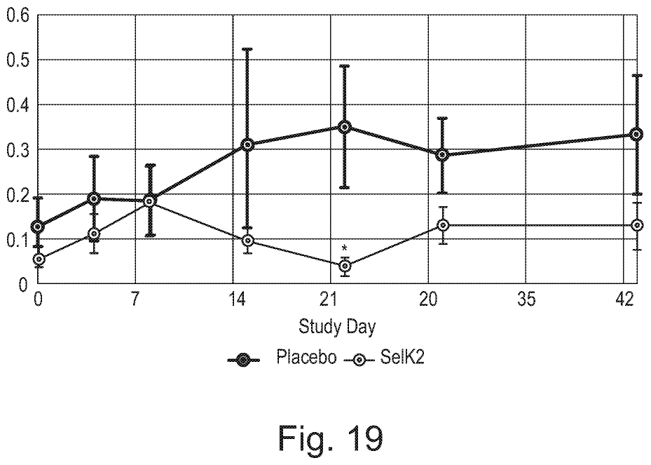

FIG. 19 shows mean (±SE) absolute differential epithelial cell count (10E6/g) in sputum of COPD patients treated with placebo or SelK2. P=0.0167

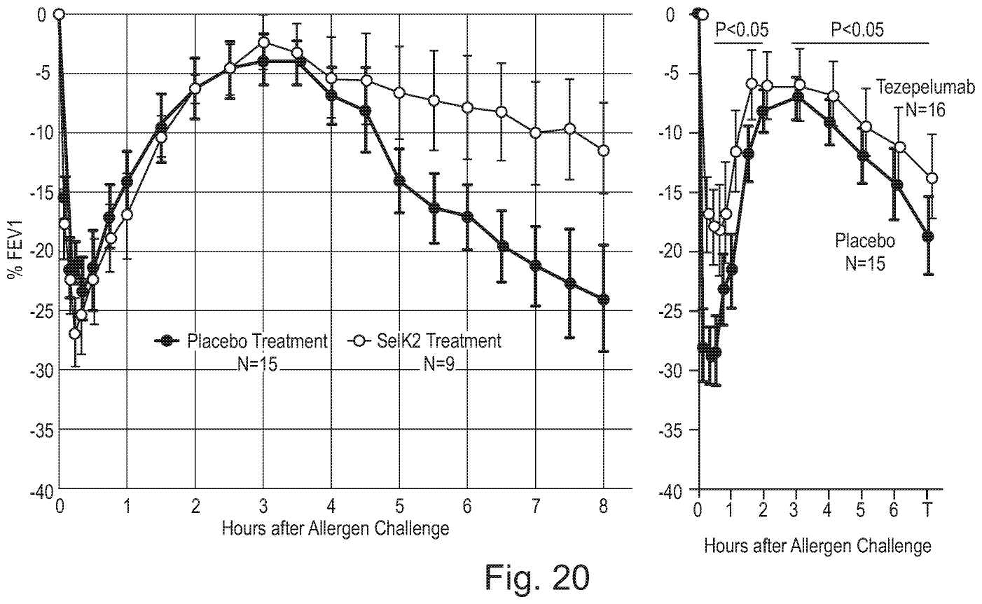

FIG. 20 Comparison of % Fall in FEV1 following allergen challenge in patients with mild asthma treated with SelK2 versus Tezepelumab.

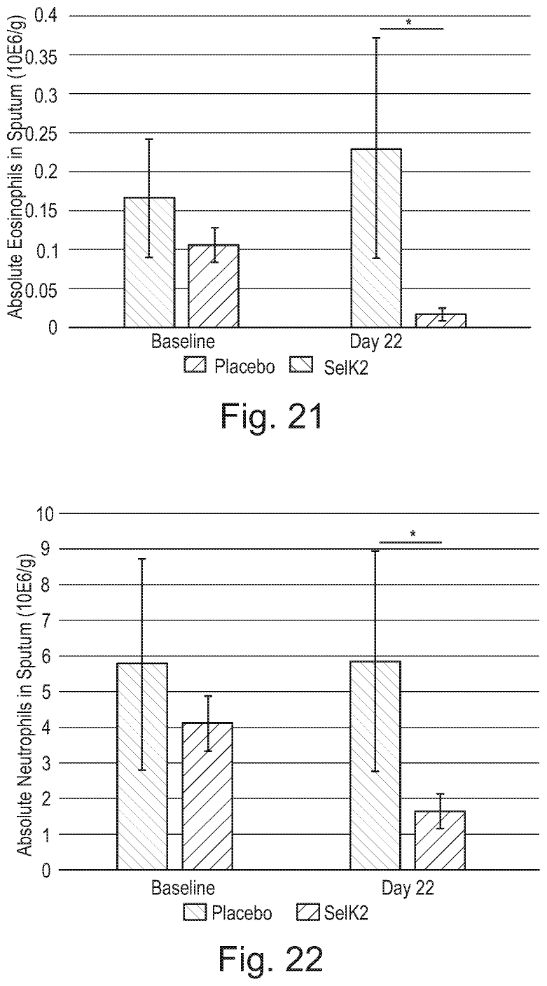

FIG. 21 shows mean (±SE) absolute numbers of eosinophils in sputum at baseline and day 22 of COPD patients treated with placebo or SelK2.

FIG. 22 shows mean (±SE) absolute numbers of neutrophils in sputum at baseline and day 22 of COPD patients treated with placebo or SelK2.

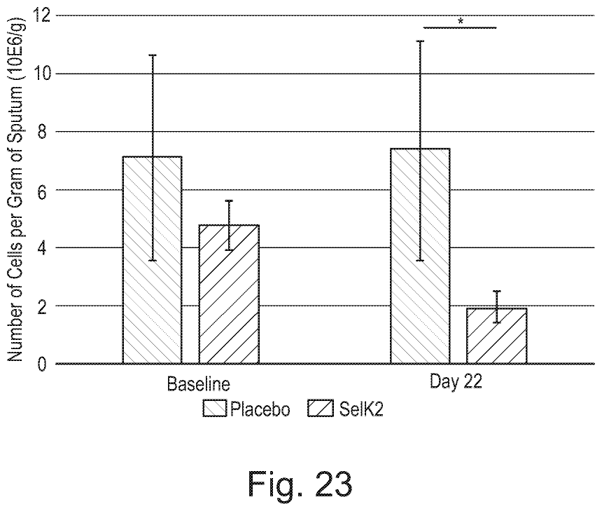

FIG. 23 shows mean (±SE) total number of cells per gram of sputum at baseline and day 22 of COPD patients treated with placebo or SelK2.

### DETAILED DESCRIPTION OF THE INVENTION

Embodiments of the present disclosure provide humanised antibodies, and antigen binding fragments thereof, that bind to PSGL-1. Potential benefits of these antibodies include the ability to bind to PSGL-1 with high affinity and the potential to provide beneficial therapeutic activity. Benefits of the disclosed antibodies may include an ability to function as blocking antibodies, which may be able to inhibit formation of complexes between PSGL-1 and P-selectin bound to one another. The antibodies disclosed may alternatively or additionally be able to disrupt complexes between PSGL-1 and P-selectin that have already formed. It will be readily appreciated that these properties may confer therapeutic utility on the antibodies disclosed, and this gives rise to methods of treatment and medical uses employing the antibodies of the invention (or antigen binding fragments thereof), and pharmaceutical compositions comprising the antibodies of the invention (or antigen binding fragments thereof).

Humanisation of antibodies is a well-established technique. However, the reduction in immunogenicity exhibited by humanised antibodies often comes at the expense of lessened affinity as compared to the parent antibody from which they are derived. Surprisingly, the antibodies of the invention demonstrate improved affinity as compared to their parent antibody, while also exhibiting low immunogenicity of the sort required for therapeutic use.

Moreover, clinical trials using the antibodies of the invention have proven that they provide surprisingly effective treatment in respect of respiratory conditions, such as asthma and COPD.

Treatments using the antibodies of the invention reduced eosinophil counts in the sputum of asthmatic patients. Moreover, lung function of asthma patients was significantly improved on treatment using the antibodies disclosed herein. Indeed, the therapeutic effect achieved was greater than that obtained using alternative agents that are currently provided for the clinical treatment of asthma, indicating that treatments using these new antibodies may constitute an improvement upon the treatments currently available.

In the case of patients with COPD, treatments using the antibodies of the invention resulted in significant decreases in the numbers of eosinophils, neutrophils and epithelial cells in sputum samples.

Furthermore, the properties of the antibodies of the invention clearly indicate that these benefits may be provided in respect of many other conditions characterised by the presence of undesirable inflammation.

Excessive or aberrant recruitment of leukocytes lies at the heart of many disorders, and particularly inflammatory diseases or conditions. Extravasation of white blood cells, such as eosinophils or neutrophils, allows their accumulation within tissues, where their presence gives rise to damaging effects. Attachment and rolling of white blood cells is a required step prior to extravasation, and PSGL-1 plays a key role in these actions. PSGL-1 on eosinophils may also bind to P-selectin on the surface of activated platelets, and this binding is able to initiate upregulation of further adhesion molecules on the eosinophils that also increase their binding to blood vessel walls and extravasation.

Up-regulation of P-selectin on endothelial cells and on platelets of patients with sickle cell disease contributes to cell-to-cell interactions involved in the pathogenesis of vaso-occlusion and pain crisis associated with this disease. Extravasation of white blood cells also plays a role in excessive inflammation that may occur in SCD.

Without wishing to be bound by any hypothesis, the ability of the antibodies of the invention, or antibodies suitable for use in the methods of treatment or medical uses of the invention, to inhibit or disrupt binding of PSGL-1 to its ligands may facilitate their ability to reduce or reverse attachment and/or rolling activity of white blood cells, thus preventing leukocyte extravasation. This impact upon a central process within the inflammatory pathway indicates that these treatments have the potential to provide therapies for a broad range of diseases or conditions associated with leukocyte recruitment and inflammation.

Similarly, the antibodies' ability to inhibit or disrupt binding of PSGL-1 to its ligands may enable these antibodies to reduce or reverse cell-to-cell interactions involved in vaso-occlusion and/or pain crisis associated with SCD. Thus, treatments using such antibodies may provide effective prevention of, or relief from, these symptoms of SCD.

The invention will now be further described with reference to the following terms and definitions.

### Antibodies of the Invention

The first aspect of the invention provides a humanised anti-PSGL-1 antibody, or an antigen binding fragment thereof, comprising at least one CDR of the antibody heavy chain set out in SEQ ID NO: 1 and at least one CDR of the antibody light chain set out in SEQ ID NO: 2.

An antibody, or an antigen binding fragment thereof, of the invention may comprise at least two CDRs of the antibody heavy chain set out in SEQ ID NO: 1. An antibody, or an antigen binding fragment thereof, of the invention may comprise the three CDRs of the antibody heavy chain set out in SEQ ID NO: 1.

An antibody, or an antigen binding fragment thereof, of the invention may comprise at least two CDRs of the antibody light chain set out in SEQ ID NO: 2. An antibody, or an antigen binding fragment thereof, of the invention may comprise the three CDRs of the antibody light chain set out in SEQ ID NO: 2.

An antibody, or an antigen binding fragment thereof, of the invention may comprise the three CDRs of the antibody heavy chain set out in SEQ ID NO: 1 and the three CDRs of the antibody light chain set out in SEQ ID NO: 2.

These antibodies of the invention, or antigen binding fragments thereof, may be employed in the pharmaceutical compositions of the third aspect of the invention. Their immunoglobulin chains may also be encoded by the polynucleotides of the fourth or fifth aspects of the invention.

An anti-PSGL-1 antibody, or an antigen binding fragment thereof, of the invention may be defined as comprising the heavy chain variable sequence set out in SEQ ID NO: 26 and the light chain variable sequence set out in SEQ ID NO: 29. In another suitable example, an anti-PSGL-1 antibody or an antigen binding fragment thereof of the invention may be defined as comprising the heavy chain sequence set out in SEQ ID NO: 1 and the light chain sequence set out in SEQ ID NO: 2.

An exemplary anti-PSGL-1 antibody, or an antigen binding fragment thereof, of the invention is defined by the heavy chain amino acid sequence set out in SEQ ID NO: 1 and the light chain amino acid sequence set out in SEQ ID NO: 2.

The heavy and light chain sequences set out in SEQ ID NO: 1 and SEQ ID NO:2 respectively contain the variable region sequences set out in SEQ ID NO: 26 and SEQ ID NO: 29.

This gives rise to the second aspect of the invention, which provides an anti-PSGL-1 antibody or an antigen binding fragment thereof:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2.

An antibody, or an antigen binding fragment thereof, in accordance with this second aspect of the invention may comprise the heavy chain variable sequence set out in SEQ ID NO: 26 and the light chain variable sequence set out in SEQ ID NO: 29, without the constant regions set out in SEQ ID NOs: 1 and 2.

An antibody, or an antigen binding fragment thereof, in accordance with this second aspect of the invention may comprise the heavy chain amino acid sequence set out in SEQ ID NO: 1 and the light chain amino acid sequence set out in SEQ ID NO: 2. An antibody in accordance with this aspect of the invention may consist of the heavy chain amino acid sequence set out in SEQ ID NO: 1 and the light chain amino acid sequence set out in SEQ ID NO: 2.

In a suitable embodiment, an antibody in accordance with the second aspect of the invention is a humanised antibody.

Antibodies or antigen binding fragments thereof in accordance with either the first or second aspects of the invention may both be referred to as “antibodies of the invention”. For the avoidance of doubt, antibodies of the invention should also be taken as encompassing antigen binding antibody fragments that meet the specified requirements, such as affinity or an ability to reduce binding of PSGL-1 to P-selectin, or other ligands. Functional properties that may be retained by suitable antigen binding fragments of antibodies are discussed further elsewhere in this specification. Unless the context requires otherwise, antigen binding fragments of antibodies may be used as suitable examples of “antibodies” in any of the aspects or embodiments of the invention described herein, whether such use is explicitly referred to or not.

### Antibodies Suitable for Use in the Methods of Treatment or Medical Uses of the Invention

The ninth and tenth aspects of the invention respectively relate to methods of treatment using anti-PSGL-1 antibodies, and medical uses of such anti-PSGL-1 antibodies. In each of these aspects of the invention, the anti-PSGL-1 antibody is defined as comprising at least one of the CDRs in the antibody heavy chain set out in SEQ ID NO: 1 and at least one of the CDRs in the antibody light chain set out in SEQ ID NO: 2.

It will be appreciated that an anti-PSGL-1 antibody of the invention represents a suitable example of an anti-PSGL-1 antibody that may be used in the methods of treatment or medical uses of the invention. Indeed, it will be appreciated that any of the anti-PSGL-1 antibodies, or antigen binding fragments thereof, of the invention described herein represent suitable examples of an anti-PSGL-1 antibody that may be used in the methods of treatment or medical uses of the invention.

Accordingly, except for where the context requires otherwise, all considerations set out in respect of an “antibody” or “antibodies” in the present disclosure should be taken as relevant to both the anti-PSGL-1 antibodies of the invention and the anti-PSGL-1 antibodies which may be used in the methods of treatment or medical uses of the invention.

Antibodies which may be used in the methods of treatment or medical uses of the invention should be taken as encompassing antibody fragments, particularly antigen binding fragments, that meet the specified requirements, as discussed in more detail elsewhere in this specification.

The exemplary anti-PSGL-1 antibody of the invention comprising or consisting of the heavy chain amino acid sequence set out in SEQ ID NO: 1 and the light chain amino acid sequence set out in SEQ ID NO: 2 may be used in the methods of treatment or medical uses of the invention.

### Anti-PSGL-1 Antibodies

The antibodies of the invention, and the antibodies suitable for use in the methods of treatment or medical uses of the invention, are all antibodies that bind specifically to human PSGL-1. Accordingly, all references to antibodies in the context of the various aspects of the present disclosure, should be taken as directed to anti-human PSGL-1 antibodies, unless the context requires otherwise.

In a suitable embodiment, an anti-PSGL-1 antibody of the invention binds to the same epitope as that bound by the antibody defined by SEQ ID NO: 1 and SEQ ID NO: 2.

In a suitable embodiment, an anti-PSGL-1 antibody suitable for use in the methods of treatment or medical uses of the invention binds to the same epitope as that bound by the antibody defined by SEQ ID NO: 1 and SEQ ID NO: 2.

### An Exemplary Anti-PSGL-1 Antibody of the Invention Suitable for Use in the Methods of Treatment and Medical Uses of the Invention

An exemplary anti-PSGL-1 antibody of the invention is referred to by the inventors as SelK2. The amino acid heavy chain sequence of this antibody is set out in SEQ ID NO: 1. The amino acid light chain sequence of this antibody is set out in SEQ ID NO: 2.

Suitably an anti-PSGL-1 antibody of the invention may comprise an amino acid sequence comprising SEQ ID NO: 1. Suitably an anti-PSGL-1 antibody of the invention may comprise an amino acid sequence comprising SEQ ID NO: 2. Suitably an anti-PSGL-1 antibody of the invention may comprise both an amino acid sequence comprising SEQ ID NO: 1 and an amino acid sequence comprising SEQ ID NO: 2.

In its second aspect, the invention provides an anti-PSGL-1 antibody:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2.

In a suitable embodiment, an antibody in accordance with this second aspect of the invention consists of the heavy chain sequence set out in SEQ ID NO: 1 and the light chain sequence set out in SEQ ID NO: 2.

In a suitable embodiment of the third aspect of the invention, there is provided a pharmaceutical composition comprising a humanised anti-PSGL-1 antibody:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2 and a pharmaceutically acceptable excipient.

In the eleventh aspect of the invention there is provided a polynucleotide sequence encoding the heavy chain variable sequence set out in SEQ ID NO: 26. Suitably, such a polynucleotide of the eleventh aspect of the invention may encode the heavy chain sequence set out in SEQ ID NO: 1. In the twelfth aspect of the invention there is provided a polynucleotide sequence encoding the light chain variable sequence set out in SEQ ID NO: 29. Suitably, such a polynucleotide of the twelfth aspect of the invention may encode the light chain sequence set out in SEQ ID NO: 2.

In the thirteenth aspect of the invention, there is provided a cell comprising a polynucleotide sequence encoding the heavy chain sequence set out in SEQ ID NO: 1 and a polynucleotide sequence encoding the light chain sequence set out in SEQ ID NO: 2.

In a suitable embodiment of the eighth aspect of the invention, there is provided a method of manufacturing a humanised anti-PSGL-1 antibody comprising the heavy chain sequence set out in SEQ ID NO: 1 and the light chain sequence set out in SEQ ID NO: 2, the method comprising expressing a polynucleotide sequence encoding the heavy chain sequence set out in SEQ ID NO: 1 and expressing a polynucleotide sequence encoding the light chain sequence set out in SEQ ID NO: 2.

In a suitable embodiment of the ninth aspect of the invention, there is provided a method of preventing or treating a disease or condition in a subject in need thereof, the method comprising providing a therapeutically effective amount of an anti-PSGL-1 antibody to the subject, wherein the antibody:
comprises the heavy chain variable sequence set out in SEQ ID NO: 26 and the light chain variable sequence set out in SEQ ID NO: 29; or
comprises the heavy chain sequence set out in SEQ ID NO: 1 and the light chain sequence set out in SEQ ID NO: 2.

In a suitable embodiment of the tenth aspect of the invention, there is provided an anti-PSGL-1 antibody:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2;

for use as a medicament.

### Alternative Anti-PSGL-1 Antibodies of the Invention Suitable for Use in the Methods of Treatment and Medical Uses of the Invention

Other anti-PSGL-1 antibodies of the invention, suitable for use in the methods of treatment and medical uses of the invention, may be defined with reference to their similarity to the reference heavy chain and light chain sequences set out in SEQ ID NOs: 1 and 2, respectively. These similarities may be defined with reference to the sequences of the variable regions of the exemplary heavy or light chains, or with reference to the sequences of the full-length heavy or light chains.

In particular, further anti-PSGL-1 antibodies of the invention, suitable for use in the methods of treatment and medical uses of the invention, may be defined with reference to their degree of identity to the amino sequences of the reference heavy chain and light chain sequences set out in SEQ ID NOs: 1 and 2, or the variable regions of these sequences (respectively set out in SEQ ID NO: 26 and SEQ ID NO: 29).

Percentage identity to a sequence, often referred to as “sequence identity”, is a measure used to quantify the degree of similarity between two biological sequences, such as DNA, RNA, or protein sequences. This measurement is expressed as a percentage and indicates how closely the sequences match each other at the same positions or residues. The skilled person will recognise that there are multiple common techniques that may be used to calculate percentage identity. Suitable techniques that may be used to calculate percentage identity include alignment of the sequences using various algorithms available for this purpose, such as BLAST (Basic Local Alignment Search Tool), ClustalW, or specialized protein alignment software like MUSCLE or MAFFT followed by counting the matching positions, determining the total aligned positions and calculating the percentage identity using the following formula: Percentage Identity=(Number of Matching Positions/Total Aligned Positions)×100.

Alternative antibodies comprising modified versions of the variable regions present in SEQ ID NOs: 1 and 2 may share one or more of the functional properties set out in this specification in respect of the antibodies defined by SEQ ID NOs: 1 and 2. For example, they may share the affinity of the antibodies defined by SEQ ID NOs: 1 and 2, and/or the ability to block PSGL-1 binding exhibited by these antibodies.

SEQ ID NO: 26 is the heavy chain variable region of the heavy chain sequence set out in SEQ ID NO: 1. In a suitable embodiment, an antibody of invention, or suitable for use in the methods of treatment or medical uses of the invention, comprises a heavy chain variable region based upon that set out in SEQ ID NO: 26.

For example, such an antibody may comprise a heavy chain variable region with an amino acid sequence that comprises no more than 10 amino acid alterations as compared to the sequence set out in SEQ ID NO: 26. Such an antibody may comprise a heavy chain variable region with an amino acid sequence that comprises no more than 9, no more than 8, no more than 7, no more than 6, no more than 5, no more than 4, no more than 3, no more than 2, or no more than 1 amino acid alteration as compared to the sequence set out in SEQ ID NO: 26.

SEQ ID NO: 29 is the light chain variable region of the light chain sequence set out in SEQ ID NO: 2. In a suitable embodiment, an antibody of invention, or suitable for use in the methods of treatment or medical uses of the invention, comprises a light chain variable region based on that set out in SEQ ID NO: 29.

For example, such an antibody may comprise a light chain variable region with an amino acid sequence that comprises no more than 10 amino acid alterations as compared to the sequence set out in SEQ ID NO: 29. Such an antibody may comprise a light chain variable region with an amino acid sequence that comprises no more than 9, no more than 8, no more than 7, no more than 6, no more than 5, no more than 4, no more than 3, no more than 2, or no more than 1 amino acid alteration as compared to the sequence set out in SEQ ID NO: 29.

In a suitable embodiment, an antibody of the invention, or suitable for use in the methods of treatment or medical uses of the invention, comprises a heavy chain variable region that shares at least 90% identity with the heavy chain variable sequence set out in SEQ ID NO: 26. In a suitable embodiment, such an antibody comprises a heavy chain variable region that shares at least 91%, at least 92%, at least 93%, at least 94%, at least 95%, at least 96%, at least 97%, at least 98%, or at least 99% identity with the heavy chain sequence set out in SEQ ID NO: 26. In such an embodiment the light chain variable sequence of the antibody may comprise or consist of that set out in SEQ ID NO: 29.

Alternatively, or additionally, a suitable antibody may comprise a light chain variable region that shares at least 90% identity with the light chain variable region sequence set out in SEQ ID NO: 29. In a suitable embodiment, such an antibody may comprise a light chain variable region that shares at least 91%, at least 92%, at least 93%, at least 94%, at least 95%, at least 96%, at least 97%, at least 98%, or at least 99% identity with the light chain variable sequence set out in SEQ ID NO: 29. Suitably the heavy chain variable sequence of such antibody may comprise or consist of that set out in SEQ ID NO: 26.

In a suitable embodiment, an antibody of the invention, or suitable for use in the methods of treatment or medical uses of the invention, comprises both heavy and light chain variable regions that respectively share at least 90% identity with the heavy chain variable sequence set out in SEQ ID NO: 26 and at least 90% identity with the light chain variable sequence set out in SEQ ID NO: 29. In a suitable embodiment, such an antibody comprises heavy and light chain variable regions that respectively share at least at least 91%, at least 92%, at least 93%, at least 94%, at least 95%, at least 96%, at least 97%, at least 98%, or at least 99% identity with the heavy chain variable sequence set out in SEQ ID NO: 26 and with the light chain variable sequence set out in SEQ ID NO: 29.

In embodiments of the antibodies defined by the preceding paragraphs, the CDRs of the antibody may be identical to those of the anti-PSGL-1 antibody defined by SEQ ID NO: 1 and SEQ ID NO: 2, and so identical to the CDRs present in the variable regions of SEQ ID NO: 26 and SEQ ID NO: 29. In such cases the non-identical residues or regions of the antibodies may be confined to the non-CDR portions of the relevant variable regions.

Thus, an antibody of the invention, or suitable for use in the methods of treatment or medical uses of the invention, may suitably comprise the CDRs of the antibody heavy chain sequence set out in SEQ ID NO: 1 and the CDRs of the antibody light chain sequence set out in SEQ ID NO: 2, and a heavy chain variable region that shares at least 90% identity with the heavy chain sequence set out in SEQ ID NO: 26 and/or a light chain variable region that shares at least 90% identity with the light chain sequence set out in SEQ ID NO: 29. In a suitable embodiment, such an antibody comprises the CDRs of SEQ ID NO: 1 and SEQ ID NO: 2, and a heavy chain variable region that shares at least 91%, at least 92%, at least 93%, at least 94%, at least 95%, at least 96%, at least 97%, at least 98%, or at least 99% identity with the sequence of the heavy chain variable region set out in SEQ ID NO: 26 and/or a light chain variable region that shares at least 91%, at least 92%, at least 93%, at least 94%, at least 95%, at least 96%, at least 97%, at least 98%, or at least 99% identity with the sequence of the light chain variable region set out in SEQ ID NO: 29.

Suitably, an antibody of the invention, or suitable for use in the methods of treatment or medical uses of the invention, may suitably comprise the CDRs of the antibody heavy chain sequence set out in SEQ ID NO: 1 and the CDRs of the antibody light chain sequence set out in SEQ ID NO: 2, and comprises both a heavy chain variable region that shares at least 90% identity with both the heavy chain variable sequence set out in SEQ ID NO: 26 and a light chain variable region that shares at least 90% identity with the light chain variable sequence set out in SEQ ID NO: 29. In a suitable embodiment, such an antibody comprises the CDRs of SEQ ID NO: 1 and SEQ ID NO: 2, and heavy and light chain variable regions that both share at least 91%, at least 92%, at least 93%, at least 94%, at least 95%, at least 96%, at least 97%, at least 98%, or at least 99% identity respectively with either the heavy chain variable region sequence set out in SEQ ID NO: 26 or the light chain variable region sequence set out in SEQ ID NO: 29.

Particular examples of heavy chain sequences that may be incorporated in antibodies in accordance with these embodiments include comprising the heavy chain variable sequences set out in SEQ ID NO: 27 and SEQ ID NO: 28. These heavy chain variable sequences are constituents of alternative antibodies of the invention produced by the inventors.

As noted above, SEQ ID NO: 26 is the heavy chain variable region of the heavy chain sequence set out in SEQ ID NO: 1. The serine residue at position 98 of SEQ ID NO: 26 constitutes a back mutation as compared to the lysine residue present in the acceptor sequence used in the generation of the humanised antibody of the invention.

SEQ ID NO: 27 corresponds to SEQ ID NO: 26 save that a further back mutation has been incorporated at position 75 of SEQ ID NO: 27. The back mutation at position 75 is a substitution of a serine residue with a proline residue.

SEQ ID NO: 28 corresponds to SEQ ID NO: 26, save that four further back mutations have been incorporated at positions 16, 18, 42 and 75 of SEQ ID NO: 28. The back mutation at position 16 is a substitution of an arginine residue with a glycine residue. The back mutation at position 18 is a substitution of a lysine residue with an arginine residue. The back mutation at position 42 is a substitution of a glycine residue with a glutamic acid residue. The back mutation at position 75 is the substitution of a serine residue with a proline residue.

SEQ ID NO: 26 includes the CDRs of SEQ ID NO: 1, and SEQ ID NOs: 27 and 28 both retain all CDRs of SEQ ID NO: 1. SEQ ID NO: 27 shares 99% identity with SEQ ID NO: 26, while SEQ ID NO: 28 shares 96% identity with SEQ ID NO: 26.

SEQ ID NO: 29 is the light chain variable region of the light chain sequence set out in SEQ ID NO: 2.

Examples of alternative light chain sequences that may be incorporated in antibodies in accordance with these embodiments include the light chain sequence set out in SEQ ID NO: 30. This sequence corresponds to that set out in SEQ ID NO: 29, save that two back mutations have been incorporated at positions 15 and 18 of SEQ ID NO: 30. The back mutation at position 15 is a substitution of a proline residue with a leucine residue. The back mutation at position 18 is a substitution of a proline residue with a glutamine residue.

SEQ ID NO: 30 retains all CDRs of SEQ ID NO: 2, and shares 98% identity with SEQ ID NO: 29.

Antibodies incorporating any of the heavy chain variable sequence of SEQ ID NO: 27, the heavy chain variable sequence of SEQ ID NO: 28, or the light chain variable sequence of SEQ ID NO: 30 may be employed in methods of treatment or in medical uses of the invention.

It will be appreciated that anti-PSGL-1 antibodies of the invention, suitable for use in the methods of treatment and medical uses of the invention, may also be defined with reference to their similarity to the full-length heavy chain and light chain sequences set out in SEQ ID NOs: 1 and 2, respectively.

In a suitable embodiment, an antibody of invention, or suitable for use in the methods of treatment or medical uses of the invention, comprises a heavy chain with an amino acid sequence that comprises no more than 10 amino acid alterations as compared to the sequence set out in SEQ ID NO: 1. Such an antibody may comprise a heavy chain with an amino acid sequence that comprises no more than 9, no more than 8, no more than 7, no more than 6, no more than 5, no more than 4, no more than 3, no more than 2, or no more than 1 amino acid alteration as compared to the sequence set out in SEQ ID NO: 1.

In a suitable embodiment, an antibody of invention, or suitable for use in the methods of treatment or medical uses of the invention, comprises a light chain with an amino acid sequence that comprises no more than 10 amino acid alterations as compared to the sequence set out in SEQ ID NO: 2. Such an antibody may comprise a light chain with an amino acid sequence that comprises no more than 9, no more than 8, no more than 7, no more than 6, no more than 5, no more than 4, no more than 3, no more than 2, or no more than 1 amino acid alteration as compared to the sequence set out in SEQ ID NO: 2.

In a suitable embodiment, an antibody of the invention, or suitable for use in the methods of treatment or medical uses of the invention, comprises a heavy chain that shares at least 90% identity with the heavy chain sequence set out in SEQ ID NO: 1. In a suitable embodiment, such an antibody comprises a heavy chain that shares at least 91%, at least 92%, least 93%, at least 94%, least 95%, at least 96%, at least 97%, at least 98%, or at least 99% identity with the heavy chain sequence set out in SEQ ID NO: 1. In such an embodiment the light chain sequence of the antibody may comprise or consist of that set out in SEQ ID NO: 2.

Alternatively, or additionally, a suitable antibody may comprise a light chain that shares at least 90% identity with the light chain sequence set out in SEQ ID NO: 2. In a suitable embodiment, such an antibody may comprise a light chain that shares at least 91%, at least 92%, at least 93%, at least 94%, at least 95%, at least 96%, at least 97%, at least 98%, or at least 99% identity with the light chain sequence set out in SEQ ID NO: 2. Suitably the heavy chain sequence of such antibody may comprise or consist of that set out in SEQ ID NO: 1.

In a suitable embodiment, an antibody of the invention, or suitable for use in the methods of treatment or medical uses of the invention, shares at least 90% identity with the heavy chain sequence set out in SEQ ID NO: 1 and at least 90% identity with the light chain sequence set out in SEQ ID NO: 2. In a suitable embodiment, such an antibody shares at least 91%, at least 92%, at least 93%, at least 94%, at least 95%, at least 96%, at least 97%, at least 98%, or at least 99% identity with both the heavy chain sequence set out in SEQ ID NO: 1 and with the light chain sequence set out in SEQ ID NO: 2.

In embodiments of the antibodies defined by the preceding paragraphs, the CDRs of the antibody may be identical to those of the anti-PSGL-1 antibody defined by SEQ ID NO: 1 and SEQ ID NO: 2, and the non-identical regions may be confined to the non-CDR portions of the antibody.

Thus, an antibody of the invention, or suitable for use in the methods of treatment or medical uses of the invention, may suitably comprise the CDRs of the antibody heavy chain sequence set out in SEQ ID NO: 1 and the CDRs of the antibody light chain sequence set out in SEQ ID NO: 2, and a heavy chain that shares at least 90% identity with the heavy chain sequence set out in SEQ ID NO: 1 and/or a light chain that shares at least 90% identity with the light chain sequence set out in SEQ ID NO: 2. In a suitable embodiment, such an antibody comprises the CDRs of SEQ ID NO: 1 and SEQ ID NO: 2, and a heavy chain that shares at least 91%, at least 92%, at least 93%, at least 94%, at least 95%, at least 96%, at least 97%, at least 98%, or at least 99% identity with the heavy chain sequence set out in SEQ ID NO: 1 and/or a light chain that shares at least 91%, at least 92%, at least 93%, at least 94%, at least 95%, at least 96%, at least 97%, at least 98%, or at least 99% identity with the light chain sequence set out in SEQ ID NO: 2.

In a suitable embodiment, an antibody of the invention, or suitable for use in the methods of treatment or medical uses of the invention, may suitably comprise the CDRs of the antibody heavy chain sequence set out in SEQ ID NO: 1 and the antibody light chain sequence set out in SEQ ID NO: 2, and shares at least 90% identity with both the heavy chain sequence set out in SEQ ID NO: 1 and with the light chain sequence set out in SEQ ID NO: 2. In a suitable embodiment, such an antibody comprises the CDRs of SEQ ID NO: 1 and SEQ ID NO: 2, and shares at least 91%, at least 92%, at least 93%, at least 94%, at least 95%, at least 96%, at least 97%, at least 98%, or at least 99% identity with both the heavy chain sequence set out in SEQ ID NO: 1 and with the light chain sequence set out in SEQ ID NO: 2.

### CDRs of Antibodies of the Invention or Antibodies for Use in the Methods of Treatment or Medical Uses

The anti-PSGL-1 antibodies of the invention, and the anti-PSGL-1 antibodies which may be used in the methods of treatment or medical uses of the invention, are defined with reference to the CDRs that they include. In particular, these antibodies are defined with reference to their inclusion of at least one of the CDRs from the reference antibody heavy chain sequence set out in SEQ ID NO: 1, and at least one of the CDRs from the reference antibody light chain sequence set out in SEQ ID NO: 2.

Suitably an antibody of the invention, or an antibody for use in the methods of treatment and medical uses of the invention, comprises a heavy chain CDR H1 present in SEQ ID NO: 1. Suitably an antibody of the invention, or an antibody for use in the methods of treatment and medical uses of the invention, comprises a heavy chain CDR H2 present in SEQ ID NO: 1. Suitably an antibody of the invention, or an antibody for use in the methods of treatment and medical uses of the invention, comprises a heavy chain CDR H3 present in SEQ ID NO: 1.

Suitably an antibody of the invention, or an antibody for use in the methods of treatment and medical uses of the invention, comprises each of CDR H1 present in SEQ ID NO: 1, a CDR H2 present in SEQ ID NO: 1, and a CDR H3 present in SEQ ID NO: 1.

Suitably an antibody of the invention, or an antibody for use in the methods of treatment and medical uses of the invention, comprises a light chain CDR L1 present in SEQ ID NO: 2. Suitably an antibody of the invention, or an antibody for use in the methods of treatment and medical uses of the invention, comprises a light chain CDR L2 present in SEQ ID NO: 2. Suitably an antibody of the invention, or an antibody for use in the methods of treatment and medical uses of the invention, comprises a light chain CDR L3 present in SEQ ID NO: 2.

Suitably an antibody of the invention, or an antibody for use in the methods of treatment and medical uses of the invention, comprises each of a CDR L1 present in SEQ ID NO: 2, a CDR L2 present in SEQ ID NO: 2, and a CDR L3 present in SEQ ID NO: 2.

Suitably an antibody of the invention, or an antibody for use in the methods of treatment and medical uses of the invention, comprises each of a CDR H1 present in SEQ ID NO: 1, a CDR H2 present in SEQ ID NO: 1, a CDR H3 present in SEQ ID NO: 1, a CDR L1 present in SEQ ID NO: 2, a CDR L2 present in SEQ ID NO: 2, and a CDR L3 present in SEQ ID NO: 2.

It will be appreciated that there are a number of different schemes and conventions by which the amino acid residues making up CDRs within an antibody heavy chain or an antibody light chain may be identified. Merely by way of example, these include the Kabat numbering scheme, the Chothia numbering scheme, and the IMGT numbering scheme.

By way of guidance to the skilled practitioner wishing to practice the present invention, Table 15 lists the CDRs of the exemplary antibody SelK2, and set out in SEQ ID NOs: 1 and 2, as identified by:
a combination of the Kabat and Chothia numbering schemes;
the Kabat numbering scheme;
the IMGT numbering scheme; and
the Chothia numbering scheme.

The CDRs that may be included in antibodies of the invention, or in antibodies suitable for use in the methods of treatment and medical uses of the invention, may suitably be selected from the sequences of SelK2 set out in Table 15.

Accordingly, a CDR H1 of an antibody of the invention, or of an antibody suitable for use in the methods of treatment and medical uses of the invention, may comprise or consist of a sequence selected from the group consisting of: SEQ ID NO: 3; SEQ ID NO: 9; SEQ ID NO: 15; and SEQ ID NO: 44. A CDR H2 of an antibody of the invention, or of an antibody suitable for use in the methods of treatment and medical uses of the invention, may comprise or consist of a sequence selected from the group consisting of: SEQ ID NO: 4; SEQ ID NO: 10; SEQ ID NO: 16; and SEQ ID NO: 45. A CDR H3 of an antibody of the invention, or of an antibody suitable for use in the methods of treatment and medical uses of the invention, may comprise or consist of a sequence selected from the group consisting of: SEQ ID NO: 5; SEQ ID NO: 11; SEQ ID NO: 17; and SEQ ID NO: 46.

A CDR L1 of an antibody of the invention, or of an antibody suitable for use in the methods of treatment and medical uses of the invention, may comprise or consist of a sequence selected from the group consisting of: SEQ ID NO: 6; SEQ ID NO: 12; SEQ ID NO: 18; and SEQ ID NO: 47. A CDR L2 of an antibody of the invention, or of an antibody suitable for use in the methods of treatment and medical uses of the invention, may comprise or consist of a sequence selected from the group consisting of: SEQ ID NO: 7; SEQ ID NO: 13; SEQ ID NO: 19; and SEQ ID NO: 48. A CDR L3 of an antibody of the invention, or of an antibody suitable for use in the methods of treatment and medical uses of the invention, may comprise or consist of a sequence selected from the group consisting of: SEQ ID NO: 8; SEQ ID NO: 14; SEQ ID NO: 20; and SEQ ID NO: 49.

Suitably, the CDRs included in an antibody of the invention, or suitable for use in the methods of treatment and medical uses of the invention, may each be selected with reference to the same numbering scheme. Alternatively, each of the CDRs of an antibody of the invention, or of an antibody suitable for use in the methods of treatment and medical uses of the invention, may be independently selected from those set out in Table 15, such that the antibody contains a CDR H1, a CDR H2, a CDR H3, a CDR L1, a CDR L2, and a CDR L3.

Suitably an antibody of the invention, or an antibody for use in the methods of treatment and medical uses of the invention, comprises one or more CDRs from SEQ ID NO: 1 and one or more CDRs from SEQ ID NO: 2 as determined with reference to a combination of the Kabat and Chothia numbering schemes.

Suitably an antibody of the invention, or an antibody for use in the methods of treatment and medical uses of the invention, in accordance with this embodiment comprises a Kabat/Chothia-defined heavy chain CDR H1 of SEQ ID NO: 3. Suitably an antibody of the invention, or an antibody for use in the methods of treatment and medical uses of the invention, in accordance with this embodiment comprises a Kabat/Chothia-defined heavy chain CDR H2 of SEQ ID NO: 4. Suitably an antibody of the invention, or an antibody for use in the methods of treatment and medical uses of the invention, in accordance with this embodiment comprises a Kabat/Chothia-defined heavy chain CDR H3 of SEQ ID NO: 5.

Suitably an antibody of the invention, or an antibody for use in the methods of treatment and medical uses of the invention, comprises each of a Kabat/Chothia-defined CDR H1 of SEQ ID NO: 3, a Kabat/Chothia-defined CDR H2 of SEQ ID NO: 4, and a Kabat/Chothia-defined CDR H3 of SEQ ID NO: 5.

Suitably an antibody of the invention, or an antibody for use in the methods of treatment and medical uses of the invention, comprises a Kabat/Chothia-defined light chain CDR L1 of SEQ ID NO: 6. Suitably an antibody of the invention, or an antibody for use in the methods of treatment and medical uses of the invention, comprises a Kabat/Chothia-defined light chain CDR L2 of SEQ ID NO: 7. Suitably an antibody of the invention, or an antibody for use in the methods of treatment and medical uses of the invention, comprises a Kabat/Chothia-defined light chain CDR L3 of SEQ ID NO: 8.

Suitably an antibody of the invention, or an antibody for use in the methods of treatment and medical uses of the invention, comprises each of a Kabat/Chothia-defined CDR L1 of SEQ ID NO: 6, a Kabat/Chothia-defined CDR L2 of SEQ ID NO: 7, and a Kabat/Chothia-defined CDR L3 of SEQ ID NO: 8.

Suitably an antibody of the invention, or an antibody for use in the methods of treatment and medical uses of the invention, comprises each of a Kabat/Chothia-defined CDR H1 of SEQ ID NO: 3, a Kabat/Chothia-defined CDR H2 of SEQ ID NO: 4, a Kabat/Chothia-defined CDR H3 of SEQ ID NO: 5, a Kabat/Chothia-defined CDR L1 of SEQ ID NO: 6, a Kabat/Chothia-defined CDR L2 of SEQ ID NO: 7, and a Kabat/Chothia-defined CDR L3 of SEQ ID NO: 8.

Suitably an antibody of the invention, or an antibody for use in the methods of treatment and medical uses of the invention, comprises one or more CDRs from SEQ ID NO: 1 and one or more CDRs from SEQ ID NO: 2 as determined with reference to the Kabat numbering scheme.

Suitably an antibody of the invention, or an antibody for use in the methods of treatment and medical uses of the invention, comprises a Kabat-defined heavy chain CDR H1 of SEQ ID NO: 9. Suitably an antibody of the invention, or an antibody for use in the methods of treatment and medical uses of the invention, comprises a Kabat-defined heavy chain CDR H2 of SEQ ID NO: 10. Suitably an antibody of the invention, or an antibody for use in the methods of treatment and medical uses of the invention, comprises a Kabat-defined heavy chain CDR H3 of SEQ ID NO: 11.

Suitably an antibody of the invention, or an antibody for use in the methods of treatment and medical uses of the invention, comprises each of a Kabat-defined CDR H1 of SEQ ID NO: 9, a CDR H2 of SEQ ID NO: 10, and a CDR H3 of SEQ ID NO: 11.

Suitably an antibody of the invention, or an antibody for use in the methods of treatment and medical uses of the invention, comprises a Kabat-defined light chain CDR L1 of SEQ ID NO: 12. Suitably an antibody of the invention, or an antibody for use in the methods of treatment and medical uses of the invention, comprises a Kabat-defined light chain CDR L2 of SEQ ID NO: 13. Suitably an antibody of the invention, or an antibody for use in the methods of treatment and medical uses of the invention, comprises a Kabat-defined light chain CDR L3 of SEQ ID NO: 14.

Suitably an antibody of the invention, or an antibody for use in the methods of treatment and medical uses of the invention, comprises each of a Kabat-defined CDR L1 of SEQ ID NO: 12, a Kabat-defined CDR L2 of SEQ ID NO: 13, and a Kabat-defined CDR L3 of SEQ ID NO: 14.

Suitably an antibody of the invention, or an antibody for use in the methods of treatment and medical uses of the invention, comprises each of a Kabat-defined CDR H1 of SEQ ID NO: 9, a Kabat-defined CDR H2 of SEQ ID NO: 10, a Kabat-defined CDR H3 of SEQ ID NO: 11, a Kabat-defined CDR L1 of SEQ ID NO: 12, a Kabat-defined CDR L2 of SEQ ID NO: 13, and a Kabat-defined CDR L3 of SEQ ID NO: 14.

Suitably an antibody of the invention, or an antibody for use in the methods of treatment and medical uses of the invention, comprises one or more CDRs from SEQ ID NO: 1 and one or more CDRs from SEQ ID NO: 2 as determined with reference to the IMGT numbering scheme.

Suitably an antibody of the invention, or an antibody for use in the methods of treatment and medical uses of the invention, comprises an IMGT-defined heavy chain CDR H1 of SEQ ID NO: 15. Suitably an antibody of the invention, or an antibody for use in the methods of treatment and medical uses of the invention, comprises an IMGT-defined heavy chain CDR H2 of SEQ ID NO: 16. Suitably an antibody of the invention, or an antibody for use in the methods of treatment and medical uses of the invention, comprises an IMGT-defined heavy chain CDR H3 of SEQ ID NO: 17.

Suitably an antibody of the invention, or an antibody for use in the methods of treatment and medical uses of the invention, comprises each of an IMGT-defined CDR H1 of SEQ ID NO: 15, an IMGT-defined CDR H2 of SEQ ID NO: 16, and an IMGT-defined CDR H3 of SEQ ID NO: 17.

Suitably an antibody of the invention, or an antibody for use in the methods of treatment and medical uses of the invention, comprises an IMGT-defined light chain CDR L1 of SEQ ID NO: 18. Suitably an antibody of the invention, or an antibody for use in the methods of treatment and medical uses of the invention, comprises a IMGT-defined light chain CDR L2 of SEQ ID NO: 19. Suitably an antibody of the invention, or an antibody for use in the methods of treatment and medical uses of the invention, comprises a IMGT-defined light chain CDR L3 of SEQ ID NO: 20.

Suitably an antibody of the invention, or an antibody for use in the methods of treatment and medical uses of the invention, comprises each of an IMGT-defined CDR L1 of SEQ ID NO: 18, an IMGT-defined CDR L2 of SEQ ID NO: 19, and an IMGT-defined CDR L3 of SEQ ID NO: 20.

Suitably an antibody of the invention, or an antibody for use in the methods of treatment and medical uses of the invention, comprises each of an IMGT-defined CDR H1 of SEQ ID NO: 15, an IMGT-defined CDR H2 of SEQ ID NO: 16, an IMGT-defined CDR H3 of SEQ ID NO: 17, an IMGT-defined CDR L1 of SEQ ID NO: 18, an IMGT-defined CDR L2 of SEQ ID NO: 19, and an IMGT-defined CDR L3 of SEQ ID NO: 20.

Suitably an antibody of the invention, or an antibody for use in the methods of treatment and medical uses of the invention, comprises one or more CDRs from SEQ ID NO: 1 and one or more CDRs from SEQ ID NO: 2 as determined with reference to the Chothia numbering scheme.

Suitably an antibody of the invention, or an antibody for use in the methods of treatment and medical uses of the invention, in accordance with this embodiment comprises a Chothia-defined heavy chain CDR H1 of SEQ ID NO: 44. Suitably an antibody of the invention, or an antibody for use in the methods of treatment and medical uses of the invention, in accordance with this embodiment comprises a Chothia-defined heavy chain CDR H2 of SEQ ID NO: 45. Suitably an antibody of the invention, or an antibody for use in the methods of treatment and medical uses of the invention, in accordance with this embodiment comprises a Chothia-defined heavy chain CDR H3 of SEQ ID NO: 46.

Suitably an antibody of the invention, or an antibody for use in the methods of treatment and medical uses of the invention, comprises each of a Chothia-defined CDR H1 of SEQ ID NO: 44, a Chothia-defined CDR H2 of SEQ ID NO: 45, and a Chothia-defined CDR H3 of SEQ ID NO: 46.

Suitably an antibody of the invention, or an antibody for use in the methods of treatment and medical uses of the invention, comprises a Chothia-defined light chain CDR L1 of SEQ ID NO: 47. Suitably an antibody of the invention, or an antibody for use in the methods of treatment and medical uses of the invention, comprises a Chothia-defined light chain CDR L2 of SEQ ID NO: 48. Suitably an antibody of the invention, or an antibody for use in the methods of treatment and medical uses of the invention, comprises a Chothia-defined light chain CDR L3 of SEQ ID NO: 49.

Suitably an antibody of the invention, or an antibody for use in the methods of treatment and medical uses of the invention, comprises each of a Chothia-defined CDR L1 of SEQ ID NO: 47, a Chothia-defined CDR L2 of SEQ ID NO: 48, and a Chothia-defined CDR L3 of SEQ ID NO: 49.

Suitably an antibody of the invention, or an antibody for use in the methods of treatment and medical uses of the invention, comprises each of a Chothia-defined CDR H1 of SEQ ID NO: 44, a Chothia-defined CDR H2 of SEQ ID NO: 45, a Chothia-defined CDR H3 of SEQ ID NO: 46, a Chothia-defined CDR L1 of SEQ ID NO: 47, a Chothia-defined CDR L2 of SEQ ID NO: 48, and a Chothia-defined CDR L3 of SEQ ID NO: 49.

Suitably an anti-PSGL-1 antibody of the invention, or an anti-PSGL-1 antibody suitable for use in the methods of treatment and medical uses of the invention is an antibody comprising:
at least one, two or three CDRs having a sequence selected from the group consisting of: SEQ ID NO: 3; SEQ ID NO: 4; and SEQ ID NO: 5; and at least one, two or three CDRs having a sequence selected from the group consisting of: SEQ ID NO: 6; SEQ ID NO: 7; and SEQ ID NO: 8; or
at least one, two or three CDRs having a sequence selected from the group consisting of: SEQ ID NO: 9; SEQ ID NO: 10; and SEQ ID NO: 11; and at least one, two or three CDRs having a sequence selected from the group consisting of: SEQ ID NO: 12; SEQ ID NO: 13; and SEQ ID NO: 14; or
at least one, two or three CDRs having a sequence selected from the group consisting of: SEQ ID NO: 15; SEQ ID NO: 16; and SEQ ID NO: 17; and at least one, two or three CDRs having a sequence selected from the group consisting of: SEQ ID NO: 18; SEQ ID NO: 19; and SEQ ID NO: 20; or
at least one, two or three CDRs having a sequence selected from the group consisting of: SEQ ID NO: 44; SEQ ID NO: 45; and SEQ ID NO: 46; and at least one, two or three CDRs having a sequence selected from the group consisting of: SEQ ID NO: 47; SEQ ID NO: 48; and SEQ ID NO: 49.

### Humanised Antibodies of the Invention

The antibodies of the invention may be humanised anti-PSGL-1 antibodies, or antigen binding fragments thereof. Antibodies for use in the methods of treatment or medical uses of the invention may also be humanised anti-PSGL-1 antibodies.

In a suitable embodiment, a humanised antibody comprises the CDRs set out in the heavy chain of SEQ ID NO: 1 and in the light chain of SEQ ID NO: 2 and human germline acceptor sequences.

In a suitable embodiment, the heavy chain of a humanised antibody of the invention comprises the CDRs set out in SEQ ID NO: 1 in the human germline acceptor sequence VH3 1-3 3-30.5 (SEQ ID NO: 23). Suitably the framework 4 region of the heavy chain of such an antibody may be that of human germline heavy joining sequence JH4a.

In a suitable embodiment, light chain of a humanised antibody of the invention comprises the CDRs set out in SEQ ID NO: 2 in the human germline acceptor sequence IGKV2-40*01 (SEQ ID NO: 24). Suitably the framework 4 region of the light chain of such an antibody may be that of human germline kappa joining sequence JK5

For the avoidance of doubt, the murine parent antibody does not constitute a humanised antibody in accordance with the present invention.

### Framework Regions of Antibodies of the Invention

An antibody of the invention may comprise a human framework acceptor sequence. In a suitable embodiment 1, 2, 3 or 4 framework sequences of the heavy chain variable region may be a human framework sequence. In a suitable embodiment 1, 2, 3 or 4 framework sequences of the light chain variable region may be a human framework sequence.

An antibody of the invention may comprise a substantially human framework acceptor that incorporates one or more back mutations. Suitably an antibody of the invention may comprise a substantially human framework acceptor that incorporates a single back mutation. An example of such a framework region is found in SEQ ID NO: 1. Here the serine residue at position 117 of SEQ ID NO: 1 constitutes a back mutation, replacing the lysine residue found in the naturally occurring human sequence.

In a suitable embodiment, an antibody of the invention comprises a heavy chain framework region FR H1 of SEQ ID NO: 32. Suitably, an antibody of the invention comprises a heavy chain framework region FR H2 of SEQ ID NO: 33. Suitably, an antibody of the invention comprises a heavy chain framework region FR H3 of SEQ ID NO: 34. Suitably, an antibody of the invention comprises a heavy chain framework region FR H4 of SEQ ID NO: 35.

Suitably an antibody of the invention comprises a FR H1 of SEQ ID NO: 32, a FR H2 of SEQ ID NO: 33, a FR H3 of SEQ ID NO: 34, and a FR H4 of SEQ ID NO: 35.

In a suitable embodiment, an antibody of the invention comprises a light chain framework region FR L1 of SEQ ID NO: 36. Suitably, an antibody of the invention comprises a light chain framework region FR L2 of SEQ ID NO: 37. Suitably, an antibody of the invention comprises a light chain framework region FR L3 of SEQ ID NO: 38. Suitably, an antibody of the invention comprises a light chain framework region FR L4 of SEQ ID NO: 39.

Suitably an antibody of the invention comprises a FR L1 of SEQ ID NO: 36, a FR L2 of SEQ ID NO: 37, a FR L3 of SEQ ID NO: 38, and a FR L4 of SEQ ID NO: 39.

In a suitable embodiment, an antibody of the invention, or an antibody suitable for use in the methods of treatment or medical uses of the invention, comprises a FR H1 of SEQ ID NO: 32, a FR H2 of SEQ ID NO: 33, a FR H3 of SEQ ID NO: 34, a FR H4 of SEQ ID NO: 35, a FR L1 of SEQ ID NO: 36, a FR L2 of SEQ ID NO: 37, a FR L3 of SEQ ID NO: 38, and a FR L4 of SEQ ID NO: 39.

The exemplary sequences of these framework regions set out in the sequence information table have been calculated using a combination of the Kabat and Chothia numbering systems. It will be appreciated that the definitions of the framework regions may alter slightly based upon alternative numbering systems. Such changes will correspond to changes in the definitions of the CDRs determined by these alternative numbering systems (as set out in Table 15), and so it may be preferred to use numbering systems consistently to determine both CDRs and framework regions.

Suitably, a humanised antibody of the invention, or for use in the methods of treatment and medical uses of the invention, comprises a heavy chain that incorporates no more than 5 back mutations. For example, such a humanised antibody may incorporate no more than 4, no more than 3, no more than 2, or no more than 2 back mutations in the heavy chain. Suitably a humanised antibody of the invention, or for use in the methods of treatment and medical uses of the invention, comprises a single back mutation in the heavy chain.

In a suitable embodiment, a humanised antibody of the invention, or for use in the methods of treatment and medical uses of the invention, comprises a back mutation in the heavy chain to introduce a serine residue adjacent to the CDR H3.

The inventors have found that back mutations introduced into the light chain of antibodies of the invention have more impact on affinity than back mutations introduced into the heavy chain. Accordingly, a suitable humanised antibody of the invention, or for use in the methods of treatment and medical uses of the invention, may comprise a light chain that incorporates no more than 5 back mutations. For example, such a humanised antibody may incorporate no more than 4, no more than 3, no more than 2, or no more than 1 back mutations in the light chain. Suitably a humanised antibody of the invention, or an antibody for use in the methods of treatment and medical uses of the invention, comprises no back mutations in the light chain.

### Variable Regions of Antibodies of the Invention

SEQ ID NO: 26 sets out the variable region of the heavy chain of SEQ ID NO: 1. An antibody of the invention, or suitable for use in the methods of treatment or medical uses of the invention, may comprise a heavy chain comprising a variable sequence as set out in SEQ ID NO: 26. As referred to above, the serine residue at position 98 of SEQ ID NO: 26 constitutes a back mutation.

In a suitable embodiment, an antibody of the invention, or an antibody suitable for use in the methods of treatment or medical uses of the invention, comprises a heavy chain variable region of sharing at least 90%, at least 91%, least 92%, at least 93%, least 94%, at least 95%, least 96%, at least 97%, least 98%, or at least 99% identity with SEQ ID NO: 26. In a suitable embodiment, an antibody of the invention, or an antibody suitable for use in the methods of treatment or medical uses of the invention, comprises a heavy chain variable region of SEQ ID NO: 26. Such antibodies may retain the serine residue at position 98 of SEQ ID NO: 26.

SEQ ID NO: 29 sets out the variable region of the light chain of SEQ ID NO: 2. An antibody of the invention, or suitable for use in the methods of treatment or medical uses of the invention, may comprise a light chain comprising a variable sequence as set out in SEQ ID NO: 29.

In a suitable embodiment, an antibody of the invention, or an antibody suitable for use in the methods of treatment or medical uses of the invention, comprises a light chain variable region of sharing at least 90%, at least 91%, least 92%, at least 93%, least 94%, at least 95%, least 96%, at least 97%, least 98%, or at least 99% identity with SEQ ID NO: 29. In a suitable embodiment, an antibody of the invention, or an antibody suitable for use in the methods of treatment or medical uses of the invention, comprises a light chain variable region of SEQ ID NO: 26.

In a suitable embodiment, an antibody of the invention, or an antibody suitable for use in the methods of treatment or medical uses of the invention, comprises a heavy chain variable region of SEQ ID NO: 26, and a light chain variable region of SEQ ID NO: 29.

Further details of suitable antibodies incorporating specific examples of modifications in the sequence of the variable region are described elsewhere in the specification.

### Constant Regions of Antibodies of the Invention

An antibody of the invention, or suitable for use in the methods or uses of the invention, may comprise any desired constant region. A skilled person will readily be able to select a suitable constant region taking into account the requirements of the applications in which the antibody is to be used.

Merely by way of example, a suitable antibody may comprise a human IgG2 heavy chain constant region. A suitable antibody may comprise a human IgG2 heavy chain constant region of SEQ ID NO: 40.

The incorporation of a human IgG2 heavy chain constant region in the antibodies of the invention may be considered favourable for a number of reasons. For example, since the constant region of IgG2 exhibits low binding to Fc receptor, and low binding to complement component 1q (C1q), antibodies that incorporate this constant region have a reduced likelihood of inducing antibody effector functions. The human IgG2 heavy chain constant region of SEQ ID NO: 40 has particularly reduced potential for inducing such functions, since it incorporates a modification (the alanine residue at position 201 of SEQ ID NO: 40, replacing the lysine residue found at this position in naturally occurring human IgG2) that further reduces binding to C1q.

Suitably an antibody of the invention comprises a human kappa light chain constant region. For example, such an antibody may comprise a human kappa light chain constant region of SEQ ID NO: 41.

### Post-Translational Modifications

Suitably an anti-PSGL-1 antibody of the invention, or suitable for use in the methods of treatment or medical uses of the invention, may comprise one or more post-translational modifications. In a suitable embodiment, the one or more post-translational modifications may be selected from the group consisting of: glycosylation; N-terminal glutaminyl cyclization; and C-terminal lysine clipping.

In a suitable embodiment, an anti-PSGL-1 antibody of the invention, or suitable for use in the methods of treatment or medical uses of the invention, has a heavy chain that is glycosylated at Asn-289 with GOF as the major N-glycan structure.

### Fragments of Antibodies

The benefits provided by the antibodies of the invention, and those suitable for use in the methods of treatment or medical uses of the invention, may generally also be conferred by antigen binding fragments of such antibodies. Accordingly, the invention should also be taken as disclosing and relating to antigen binding fragments of the antibodies described herein.

A suitable antigen binding fragment of an antibody disclosed herein, such as an antibody of the invention or an antibody for use in the methods of treatment or medical uses of the invention, may comprise the variable region of the antibody. Indeed, such a fragment may essentially consist of the variable region of the antibody.

For the avoidance of doubt the invention provides an antigen binding fragment of a humanised antibody comprising at least one CDR of the antibody heavy chain set out in SEQ ID NO: 1 and at least one CDR of the antibody light chain set out in SEQ ID NO: 2. Such antigen binding fragments of humanised antibodies may be used in the pharmaceutical compositions of the third aspect of the invention.

Similarly, the invention provides a polynucleotide sequence encoding an antigen binding fragment of an immunoglobulin chain of a humanised antibody comprising at least one CDR of the antibody heavy chain set out in SEQ ID NO: 1 and at least one CDR of the antibody light chain set out in SEQ ID NO: 2. The invention also provides a polynucleotide sequence encoding an antigen binding fragment of an immunoglobulin chain comprising at least one CDR of the antibody heavy chain set out in SEQ ID NO: 1 and of an immunoglobulin chain comprising at least one CDR of the antibody light chain set out in SEQ ID NO: 2.

Furthermore, the invention provides cells comprising polynucleotides in accordance with these embodiments of the invention that encode antigen binding antibody fragments. Such cells may be employed in the methods of manufacture of the eighth aspect of the invention to produce antigen binding fragments of antibodies in accordance with the invention.

The methods of treatment of the ninth aspect of the invention may be practiced using antigen binding fragments of suitable antibodies. Antigen binding fragments of antibodies comprising at least one CDR of the antibody heavy chain set out in SEQ ID NO: 1 and at least one CDR of the antibody light chain set out in SEQ ID NO: 2 may be used as medicaments in accordance with the tenth aspect of the invention.

Indeed, except for where the context requires otherwise, references within the present disclosure to “an antibody of the invention” or to “antibodies the invention” should be taken as also encompassing antigen binding fragments of such antibodies.

Merely by way of example, a suitable antigen binding fragment of an antibody of the invention may be selected from the group consisting of: an antigen binding fragment (Fab); a single-domain antibody fragment (sdAb); a nanobody; a single-chain variable fragment (scFv); an antibody variable fragment (Fv); an Fd fragment; and an autonomous VH domain.

The skilled person will be well aware of many methods by which it can be determined whether or not a fragment of an antibody of the invention retains the ability to bind to the PSGL-1 antibody, thus constituting an antigen binding fragment of an antibody of the invention. Merely by way of example, the capability of an antibody fragment to bind to PSGL-1 may be determined by the methods used in the Examples to determine antibody affinity, or functional attributes of an antibody.

### Functional Properties of Antibodies of the Invention, or of Antibodies or Antibody Fragments Suitable for Use in the Methods of Treatment or Medical Uses of the Invention

In addition to the largely sequence-based and structural definitions described above, antibodies of the invention, and antibodies suitable for use in the methods of treatment and medical uses of the invention, may usefully be characterised with respect to their functional properties. These functional properties may be assessed in vitro or in vivo, as required.

The inventors have found that antibodies of the invention have functional properties that offer notable advantages in terms of their therapeutic applications.

### Affinity

Antibodies of the invention bind to PSGL-1 with high affinity.

Humanised antibodies often exhibit lessened affinity for their target as compared to the parental antibody from which they are derived, particularly when germline sequences are utilised as framework acceptors. In contrast, the inventors have demonstrated that the humanised anti-PSGL-1 antibodies of the invention have higher affinity for PSGL-1 than their parental antibody. The parental antibody from which the humanised anti-PSGL-1 antibodies of the invention are derived has an affinity constant for PSGL-1 of 16.6 nM, whereas the exemplary antibody of the invention SelK2 (comprising SEQ ID NO: 1 and SEQ ID NO: 2) has an affinity constant for PSGL-1 of 4.29 nM. This represents an almost four-fold increase in affinity as compared to the parental antibody, highly unusual in respect of a humanized antibody employing germline framework acceptor sequences.

The improved affinity of antibodies of the invention is a surprising and beneficial finding. Without wishing to be bound by any hypothesis, the inventors believe that this high affinity underpins the unexpectedly effective outcomes that can be achieved by the therapeutic uses of the antibodies of the invention in methods of treatment or medical uses of the invention.

In suitable embodiments the high affinity of the antibodies of the invention, and the antibodies suitable for use in the methods of treatment and medical uses of the invention described herein, means that these antibodies are able to not only inhibit binding of PSGL-1 to its ligands, but also to disrupt such binding that has already occurred. As described further below, this opens new therapeutic windows in which treatments can achieve benefit even when a response driven binding of PSGL-1 to its ligand has already begun.

In a suitable embodiment, an antibody of the invention, or for use in the methods of treatment and medical uses of the invention, may have an affinity constant for PSGL-1 of below 16 nM. For example, an antibody of the invention may have an affinity constant for PSGL-1 of below 15 nM, of below 14 nM, of below 13 nM, of below 12 nM, of below 11 nM, of below 10 nM, of below 9 nM, of below 8 nM, of below 7 nM, of below 6 nM, or an affinity constant of below 5 nM. Suitably, an antibody of the invention may have an affinity constant for PSGL-1 of approximately 4 nM, for example an affinity constant for PSGL-1 of 4.29 nM.

In a suitable embodiment, an antibody of the invention, or for use in the methods of treatment and medical uses of the invention, may have even greater affinity for PSGL-1, for example an affinity constant of 4 nM or less, of 2 nM or less, or of 1 nM or less.

While the high affinities of the antibodies of the invention make them particularly suitable for practicing the methods of treatment of the invention, or the medical uses of the invention, it will be recognised that, in an alternative embodiment, these methods or uses may also potentially be practiced using anti-PSGL-1 antibodies that possess a lower affinity.

The affinity of an antibody of the invention may be determined by any suitable method known to one skilled in the art. Merely by way of example, affinity, such as the affinity values set out in the paragraph above, may be derived by Surface Plasmon Resonance (SPR) using a series of escalating concentrations through saturation. Details of suitable techniques, and the results achieved using these in respect of the antibodies of the invention are set out in the Examples.

A suitable antigen binding fragment of an antibody may have affinity in accordance with any of the parameters set out above.

### Inhibition or Disruption of Binding of PSGL-1 to its Ligands

As demonstrated in the Examples, antibodies of the invention are able to inhibit and/or to disrupt binding of PSGL-1 to its ligands.

In the context of the present invention, “inhibition” of binding of PSGL-1 to a ligand may be taken as referring to the prevention of the formation of new interactions between PSGL-1 and the ligand in question.

On the other hand, “disruption” of binding of PSGL-1 to a ligand may be taken as referring to the breaking down of previously formed interactions between PSGL-1 and the ligand in question.

As discussed further elsewhere in this disclosure, these capabilities of the antibodies of the invention, and of antibodies used in the methods of treatment or medical uses of the invention, are well suited to both prevention of diseases or conditions caused by deleterious binding of PSGL-1 to its ligands, and to the treatment of diseases or conditions caused by deleterious binding of PSGL-1 to its ligands.

Generally, antibodies of the invention, or antibodies suitable for use in the methods of treatment or medical uses of the invention, may be able to inhibit or disrupt binding of PSGL-1 to a ligand by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The skilled person will readily be able to identify suitable assays for determining an extent of inhibition or disruption of binding achieved. The skilled person will recognise that there are multiple common techniques that may be used to determine the extent of inhibition or disruption of binding achieved. Suitable techniques that may be used to determine the extent of inhibition or disruption of binding achieved include Enzyme-Linked Immunosorbent Assay (ELISA), Surface Plasmon Resonance (SPR), flow cytometry, western blotting, immunoprecipitation (IP) and competition binding assays. These may be selected with reference to particular ligands of interest (for example, binding of PSGL-1 to P-selectin or to a chemokine), or with reference to particular binding-associated activities of interest (for example, cell rolling or extravasation).

A suitable antigen binding fragment of an antibody may have the ability to inhibit or disrupt binding of PSGL-1 to its ligands in accordance with any of the parameters set out above.

### Inhibition or Disruption of Binding to Selectins

PSGL-1 is known to bind to selectins including those selected from the group consisting of: P-selectin, E-selectin and L-selectin.

As set out in the Examples, the inventors have demonstrated that the antibodies of the invention are able to inhibit or disrupt binding of PSGL-1 to specific selectin ligands, in particular P-selectin and L-selectin. Inhibition or disruption of such binding will prevent or reduce selectin-mediated biological activities.

Suitably, antibodies of the invention, or antibodies suitable for use in the methods of treatment or medical uses of the invention, may be able to inhibit or disrupt binding of PSGL-1 to a selectin (such as P-selectin or L-selectin) by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to inhibit or disrupt binding of PSGL-1 to a selectin ligand (such as P-selectin or L-selectin) may be investigated by any appropriate means. For instance, this may be assessed by cell-based assays, of the sort discussed in Examples 4 or 6.

A suitable antigen binding fragment of an antibody may have the ability to inhibit or disrupt binding of PSGL-1 to selectins in accordance with any of the parameters set out above.

### Inhibition or Disruption of Binding to Chemokines

PSGL-1 is known to bind to chemokines including those selected from the group consisting of: CCL27; CCL19; and CCL21.

As set out in the Examples, the inventors have demonstrated that the antibodies of the invention are able to inhibit or disrupt binding of PSGL-1 to chemokine ligands, as exemplified by CCL27. Inhibition or disruption of such binding will prevent or reduce chemokine-mediated biological activities.

Suitably, antibodies of the invention, or antibodies suitable for use in the methods of treatment or medical uses of the invention, may be able to inhibit or disrupt binding of PSGL-1 to a chemokine (such as CCL27) by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to inhibit or disrupt binding of PSGL-1 to a chemokine ligand (such as CCL27) may be investigated by any appropriate means. For instance, this may be assessed by a SPR-based assay, of the sort discussed in Example 5.

A suitable antigen binding fragment of an antibody may have the ability to inhibit or disrupt binding of PSGL-1 to chemokines in accordance with any of the parameters set out above.

### Inhibition or Disruption of Adhesion of Cells

Interaction of PSGL-1 with its ligands contributes to increased cell adhesion through a number of different mechanisms. Attachment of PSGL-1 on white blood cells to ligands on the blood vessel wall is known to be an essential step in the adhesion of cells to substrates such as the endothelial lining of the circulation or platelets. Interaction of PSGL-1 on cells such as eosinophils with P-selectin on the surface of activated platelets also leads to elevated expression of adhesion molecules by the leukocytes, which increases their tendency to adhere. In either case, PSGL-1-mediated adhesion of leukocytes, such as eosinophils, may then be further followed by other activities associated with cell migration or extravasation.

As set out in the Examples, the inventors have demonstrated that the antibodies of the invention are able to inhibit or disrupt binding of PSGL-1 to its ligands that is associated with cell adhesion. Inhibition or disruption of such binding will prevent or reduce cell adhesion, thus preventing or reducing biological activities that occur as a result of such cell adhesions.

The cells may be leukocytes. The cells may be epithelial cells. The cells may be selected from the group consisting of: eosinophils; neutrophils; and epithelial cells. The biological activities that will be prevented or reduced by inhibition or disruption of cell adhesion may be determined with reference to the cell type in question.

Suitably, antibodies of the invention, or antibodies suitable for use in the methods of treatment or medical uses of the invention, may be able to inhibit or disrupt PSGL-1-mediated cell adhesion by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to inhibit or disrupt cell adhesion mediated by binding of PSGL-1 may be investigated by any appropriate means. For instance, this may be assessed by assays of the sort discussed in Examples 4 or 6.

A suitable antigen binding fragment of an antibody may have the ability to inhibit or disrupt adhesion of cells in accordance with any of the parameters set out above.

### Inhibition or Disruption of Cell Rolling

Interaction of PSGL-1 with its ligands is known to contribute to the process of cell rolling exhibited by cells in the early stages of adhesion to substrates such as the endothelial lining of the circulation. This rolling may be associated with activation, and/or with subsequent migration or extravasation, of the rolling cells.

As set out in the Examples, the inventors have demonstrated that the antibodies of the invention are able to inhibit or disrupt rolling of cells mediated by binding of PSGL-1 to its ligands. Inhibition or disruption of such cell rolling will prevent or reduce “down-stream” activities, such as cell extravasation.

As before, the cells rolling of which is to be inhibited or disrupted may be leukocytes. The cells may be non-leukocytic cells, such metastatic cancer cells. The cells may be selected from the group consisting of: neutrophils; eosinophils; and metastatic cells. The biological activities that will be prevented or reduced by the inhibition or disruption of cell rolling may be determined with reference to the cell type in question.

Suitably, antibodies of the invention, or antibodies suitable for use in the methods of treatment or medical uses of the invention, may be able to inhibit or disrupt PSGL-1-mediated cell rolling by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to inhibit or disrupt cell adhesion mediated by binding of PSGL-1 may be investigated by any appropriate means. For instance, this may be assessed by assays such as the neutrophil rolling assay described in Example 4.

A suitable antigen binding fragment of an antibody may have the ability to inhibit or disrupt cell rolling in accordance with any of the parameters set out above.

### Inhibition or Disruption of Cell Extravasation

Binding of cells, such as to the endothelial lining of the circulation system, via the interaction of PSGL-1 with its ligands, is a necessary step in the process of cell extravasation. Extravasated cells contribute to inflammatory responses within tissues that can have deleterious effects upon the tissue in question.

As set out in the Examples, the inventors have demonstrated that the antibodies of the invention are able to inhibit or disrupt cell extravasation mediated by binding of PSGL-1 to its ligands. Inhibition or disruption of such cell extravasation is thus able to prevent or reduce inflammation that is mediated by the extravasated cells.

The extravasation inhibited or disrupted may be leukocyte extravasation, or may be extravasation of other cells, such as metastatic cancer cells. The cells may be selected from the group consisting of: eosinophils; neutrophils; and metastatic cancer cells. The biological activities that will be prevented or reduced by the inhibition or disruption of cell rolling may be determined with reference to the cell type in question. In the case of leukocytes, this will typically bring about a reduction in inflammation, in the case of metastatic cells it may bring about a reduction in cancer metastasis.

Suitably, antibodies of the invention, or antibodies suitable for use in the methods of treatment or medical uses of the invention, may be able to inhibit or disrupt cell extravasation by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to Inhibit or disrupt cell extravasation mediated by binding of PSGL-1 may be investigated by any appropriate means. For instance, this may be assessed by assays such as those described in Example 8.

Importantly, the inventors' results demonstrate that a reduction in extravasation of leukocytes (such as eosinophils or neutrophils) on treatment with an antibody of the invention is not associated with a reduction in the number of the leukocytes present in the circulation. This indicates that the reduction in extravasation is not simply a function of reduced circulating leukocyte numbers, but is specifically associated with a reduction in the numbers of leukocytes (such as eosinophils or neutrophils) extravasating from the blood stream into the underlying tissues during the inflammatory response.

That circulating leukocyte numbers are maintained is important, as these cells have important roles in implementing appropriate, rather than aberrant, immune responses. Merely by way of example, circulating eosinophils play an important role in the body's response to parasites, and neutrophils have a key role in the innate immune response.

Accordingly, the antibodies, methods of treatment, or medical uses of the invention may be used to therapeutically reduce numbers of extravasated leukocytes (such as eosinophils or neutrophils) without adversely impacting a recipient's immunity.

A suitable antigen binding fragment of an antibody may have the ability to inhibit or disrupt cell extravasation in accordance with any of the parameters set out above.

### Reduction of Cell Numbers in Sputum

As noted below, the antibodies of the invention, and the methods of treatment and medical uses of the invention, are of particular interest in the treatment of respiratory conditions or diseases, such as asthma or COPD.

Respiratory conditions or diseases may be associated with the accumulation of cells within the sputum. This accumulation may arise as a result of cell extravasation (for example, in the case of leukocytes) or as a result of shedding of cells (for example in the case of epithelial cells lining the respiratory tract). Accordingly, the ability of an antibody to bring about a reduction in the number of cells in the sputum may provide an indication that the antibody is alleviating the respiratory condition in a manner suitable achieve a therapeutic effect. The reduction may be noted in respect of any suitable cells of interest, such as those selected from the group consisting of: eosinophils; neutrophils; and epithelial cells.

The inventors have found that the antibodies of the invention are able to reduce the number of cells accumulating in sputum. This reduction may provide a useful proxy for assessment of disease severity or progression.

Suitably, antibodies of the invention, or antibodies suitable for use in the methods of treatment or medical uses of the invention, may be able to reduce cell numbers in sputum by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The ability of an antibody to inhibit or disrupt cell extravasation mediated by binding of PSGL-1 may be investigated by any appropriate means. For instance, this may be assessed by assays such as the absolute differential eosinophil count or percentage differential eosinophil count studies described in Example 8, or the absolute differential eosinophil count or percentage differential eosinophil count studies described in Example 11.

A suitable antigen binding fragment of an antibody may have the ability to bring about a reduction in cell numbers in sputum in accordance with any of the parameters set out above.

### Improvement in Pulmonary Function Test

Perhaps the most important finding from the inventors' studies undertaken in human subjects as part of a clinical trial is that antibodies of the invention are able to bring about an improvement in lung function as demonstrated by improvement in a pulmonary function test.

In particular, the inventors have demonstrated that asthma patients receiving treatment using an antibody of the invention achieve a dramatic and statistically significant improvement in forced expiratory volume (FEV1). FEV1 provides a measurement of the maximum volume of air that an individual can forcefully exhale in one second. A higher FEV1 value indicates better lung function, and a lower FEV1 value is indicative of poorer lung function.

A subject or patient's FEV1 may be measured at baseline (before treatment with an experimental agent or placebo) and after treatment. These values may then be compared to allow the calculation of a percentage change in FEV1 (for example the maximum fall in FEV1) or to allow calculation of area under the curve (AUC) in respect of FEV1 values obtained. Either of these statistical values may be useful in determining the effect of an antibody on improvement of pulmonary function.

As set out in Example 8, asthma patients challenged with an appropriate allergen exhibit a fall in FEV1, associated with obstruction of the airways. Treatment with an antibody of the invention (SelK2), which embodies a method of treatment or medical use in accordance with the invention, results in a reduction in the fall in FEV1 during the late allergen response (LAR; 3 to 8 hours following allergen challenge). This indicates that lung function in those asthma patients receiving treatment with an antibody of the invention is improved as compared to control patients receiving placebo. This is observed in relation to both the area under the curve for % FEV1 fall, and the maximum % fall in FEV1. The change in respect of maximum % fall indicates a statistically significant improvement in lung function in subjects receiving treatment with the antibody of the invention.

An antibody of the invention, or an antibody suitable for use in the methods of treatment or medical uses of the invention, may be able to improve lung function by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or even by 100%.

The Examples demonstrated that suitable embodiment of the antibodies of the invention, or of antibodies for use in the methods of treatment or medical uses of the invention, are able to improve lung function in asthmatic patients by at least 35% as compared to placebo. Indeed, such antibodies are able to improve lung function in asthmatic patients by at least 50% as compared to placebo. These changes are greater than those that can be achieved using currently available therapies, and so demonstrate the improvements in treatment that are made available to patients with lung diseases by the inventors' developments.

Suitably the improvement in lung function may be demonstrated in an asthma patient undergoing allergen challenge with respect to an improvement in the maximum % fall in FEV1 as compared to placebo control. Suitably such an improvement may occur during the LAR.

A suitable antigen binding fragment of an antibody may have the ability to bring about an improvement in a pulmonary function test in accordance with any of the parameters set out above.

### Low Immunogenicity of Antibodies of the Invention or Suitable for Use in the Methods of Treatment or Medical Uses of the Invention

It is generally recognised that therapeutic antibodies may give rise to immune responses within their recipients. This is particularly problematic in the case of therapeutic antibodies that need to be administered for prolonged periods of time, such as in the treatment of chronic conditions or diseases.

Even humanised antibodies may trigger an immune response in this manner. Immune responses to humanised therapeutic antibody result in the production of human anti-human antibodies (HAHAs). HAHAs bind to the therapeutic antibody, and may interfere with its ability to bind to its corresponding antigen (such as PSGL-1). Consequently, the presence of HAHAs can in some cases significantly decrease the therapeutic effectiveness of an antibody.

Accordingly, it is advantageous if therapeutic antibodies demonstrate low immunogenicity, thus decreasing the risk of HAHA formation and loss of therapeutic effect.

The antibodies of the invention, as exemplified by SelK2, have demonstrated low immunogenicity in recipients. As described in Example 10 and shown in Table 13, immunogenicity analysis conducted as part of a clinical trial using SelK2 showed that responses were of low levels, some of which did not repeat at later time points, and no neutralisation of the activity of the antibody of the invention was observed. Furthermore, some patients receiving placebo were also noted to demonstrate low level responses by the assay method used.

Accordingly, it will be recognised that the antibodies of the invention demonstrate low immunogenicity, and that this makes them well suited to therapeutic use in practice, and particularly to use in the treatment of chronic conditions or diseases.

A suitable antigen binding fragment of an antibody may have low immunogenicity in accordance with any of the parameters set out above.

### Methods of Treatment and Medical Uses

As noted above, the antibodies of the invention have properties that make them suitable for therapeutic use in a number of contexts. These properties give rise to the methods of treatment using the antibodies of the invention, and to medical uses of the antibodies of the invention, more details of which are provided below.

Given these beneficial properties of the antibodies of the invention, and in view of the relationship between the amino acid sequence, structure and function of antibodies, it will be appreciated that antibodies sharing sequence characteristics with the antibodies of the invention may also be employed in corresponding methods of treatment and in medical uses. In particular, antibodies that comprise sequences in common with those of the antibodies of the invention, and hence share binding characteristics with those of the antibodies of the invention, may be employed in methods of treatment and medical uses of this sort. Accordingly, the methods or medical uses of the invention may employ not only the antibodies of the invention, but also antibodies that comprise at least one CDR of the antibody heavy chain set out in SEQ ID NO: 1 and at least one CDR of the antibody light chain set out in SEQ ID NO: 2. Specific embodiments of such antibodies have been considered at length above.

### Method of Treatment/Medical Use Protection

According to a ninth aspect of the invention, there is provided a method of preventing or treating a disease or condition in a subject in need thereof, the method comprising providing a therapeutically effective amount of an anti-PSGL-1 antibody to the subject, wherein the antibody comprises at least one CDR of the antibody heavy chain set out in SEQ ID NO: 1 and at least one CDR of the antibody light chain set out in SEQ ID NO: 2.

The antibody provided in a method of the ninth aspect of the invention may be an anti-PSGL-1 antibody in accordance with any embodiment of the first aspect of the invention, or an anti-PSGL-1 antibody in accordance with the second aspect of the invention.

The disease or condition to be prevented or treated by a method of the ninth aspect of the invention may be a condition or disease associated with binding of PSGL-1 to its ligands. Examples of such conditions or diseases are considered below.

According to an tenth aspect of the invention, there is provided an anti-PSGL-1 antibody comprising at least one CDR of the antibody heavy chain set out in SEQ ID NO: 1 and at least one CDR of the antibody light chain set out in SEQ ID NO: 2, for use as a medicament. The antibody may be an anti-PSGL-1 antibody in accordance with any embodiment of the first aspect of the invention, or an anti-PSGL-1 antibody in accordance with the second aspect of the invention.

Antibodies for use in accordance with the tenth aspect of the invention, may be for use in the prevention or treatment of diseases or conditions associated with binding of PSGL-1 to its ligands.

Antibodies for use in accordance with the tenth aspect of the invention may be used in the form of a pharmaceutical composition comprising the antibody and a pharmaceutically acceptable excipient. Such compositions may also be used to provide the requisite antibodies in the methods of treatment of the ninth aspect of the invention.

“Prevention or Treatment” of Diseases or Conditions Associated with Binding of PSGL-1 to its Ligands

The skilled person will be aware of a wide range of diseases or conditions that are associated with binding of PSGL-1 to its ligands. Such diseases or conditions may be caused by binding of PSGL-1 to one or more of its ligands. Examples of such diseases or conditions are considered in more detail over the following pages. The antibodies of the invention, and the methods of treatment and medical uses of the invention, may be used in the prevention and/or treatment of such diseases or conditions.

In the context of the present disclosure, references to the “prevention” of a disease or condition associated with binding of PSGL-1 to its ligands may be taken as referring to prophylactic medical intervention that is intended to stop one or more incidences of a disease or condition associated with binding of PSGL-1 to its ligands from occurring or from further developing or progressing. Antibodies may be used to prevent a disease or condition by inhibiting the formation of new binding between PSGL-1 and one or more of its ligands.

References within the present disclosure to “prevention” of a disease or condition, either generically or specifically, may be taken as encompassing delaying a symptom or onset of the relevant disease or condition. Furthermore, references within the present disclosure to “prevention” of a disease or condition, either generically or specifically, should also be taken as also encompassing incomplete or partial prevention of the relevant disease or condition. Thus, prevention may result in the complete avoidance of symptoms of a disease or condition (for example such that asthma symptoms do not occur on exposure to an allergen), or partial avoidance of symptoms of a disease or condition (for example, such that symptoms experienced on exposure to an allergen are reduced or delayed).

In the case of treatment of a respiratory disease or condition such as asthma or COPD, prevention of the disease or condition may be demonstrated by improved lung function as compared to the function that would be achieved in the absence of treatment. As discussed in more detail elsewhere in this specification, lung function may be assessed with reference to changes in FEV1 in response to allergen challenge achieved with or without treatment. Lung function may be improved by at least 20%, at least 30%, at least 40%, at least 50%, or more, as compared to the function that would occur in the absence of treatment.

Alternatively, or additionally, in prevention of a respiratory disease or condition such as asthma or COPD, prevention of the disease may be demonstrated by a reduction of the number of localised inflammatory cells (such as eosinophils or neutrophils) present as compared to the numbers of such cells that would be present in the absence of treatment. It has been reported that the number of localised inflammatory cells present in the lungs of patients with respiratory diseases or conditions such as asthma or COPD correlates with disease severity. The quantity of localised inflammatory cells present in the lungs may be assessed with reference to the number of such cells present in sputum, as considered elsewhere in this specification. The number of localised inflammatory cells present in the lungs may be reduced by at least 20%, at least 30%, at least 40%, at least 50%, at least 60%, or more, as compared to the number of such cells present in sputum in the absence of treatment.

In methods of treatment or medical uses in accordance with such embodiments of the invention, a concentration of an antibody may be maintained in the circulation that is sufficient to achieve at least 30%, at least 40%, at least 50%, at least 60%, at least 70%, at least 80% or at least 90% inhibition of binding of PSGL-1 to its ligands. This approach may thus prevent the steps of leukocyte adhesion, rolling and extravasation (particularly in respect of eosinophils) that may otherwise contribute to a disease or condition (such as an asthma attack). Suitable routes of administration and dosing regimens may be selected with this aim in mind.

References to “treatment” of diseases or condition associated with binding of PSGL-1 to its ligands may be taken as referring to medical intervention that is intended to alleviate one or more symptoms that are already occurring in respect of a disease or condition associated with binding of PSGL-1 to its ligands (as contrasted with the prophylactic preventative uses described above). “Treatment” in this context may include complete treatment, such that all symptoms are fully alleviated, or partial treatment (in which symptoms are incompletely alleviated). Partial treatment, in which symptoms are alleviated by at least 20%, at least 40%, at least 60%, or at least 80% as compared to those occurring without treatment, may be sufficient to provide valuable relief to a subject.

Without wishing to be bound by any hypothesis, antibodies may be used to treat a disease or condition by disrupting existing binding between PSGL-1 and one or more of its ligands.

For example, in methods of treatment or medical uses in accordance with these embodiments of the invention, a concentration of an antibody may be established in the circulation that is sufficient to disrupt binding of PSGL-1 to its ligands. This approach may thus break down existing binding, associated with an ongoing disease or condition (such as an asthma attack in progress). Disrupting binding in this manner is able to reverse leukocyte adhesion and rolling (for example in respect of eosinophils) that is in progress and prevent further extravasation associated with the condition or disease. Suitable routes of administration and dosing regimens may be selected with this aim in mind.

The antibodies, methods of treatment or medical uses of the invention may be of benefit in the prevention or treatment of a disease or condition requiring inhibition or disruption of PSGL-1-mediated binding between leukocytes (such as eosinophils) and endothelial cells.

The antibodies, methods of treatment or medical uses of the invention may be of benefit in the prevention or treatment of a disease or condition requiring inhibition or disruption of PSGL-1-mediated binding between leukocytes (such as eosinophils) and platelets.

The antibodies, methods of treatment or medical uses of the invention may be of benefit in the prevention or treatment of a disease or condition requiring inhibition or disruption of PSGL-1-mediated binding between endothelial cells and sickled erythrocytes.

### Subjects or Patients

The methods of treatment of the invention are practiced in respect of subjects requiring the prevention or treatment a disease or condition. The words “subject” and “patient” may be used interchangeably in the context of the present disclosure.

Suitably, a subject or patient may require prevention or treatment in respect of a disease or condition that it associated with binding of PSGL-1 to its ligands.

The subject or patient may have symptoms of a disease or condition requiring treatment, or may be identified as being at elevated risk of developing a disease requiring prevention.

In a suitable embodiment, a subject or patient may be subject to an acute disease or condition. In this case a method or medical use of the invention may be used to treat the disease, thereby alleviating the symptoms of the acute disease or condition. Such treatments may make use of the ability of suitable antibodies to disrupt existing binding of PSGL-1 and its ligands.

In a suitable embodiment, a subject or patient may be subject to chronic disease. In such embodiments, a method of treatment or medical use of the invention may be utilised to prevent the worsening of the chronic disease, or incidences of acute disease. Such therapeutic prevention may make use of the ability of suitable antibodies to inhibit new binding occurring between PSGL-1 and its ligands.

### Conditions to be Treated

The binding of PSGL-1 with its ligands, such as P-selectin and L-selectin, is associated with many biological processes. Accordingly, the ability to inhibit or disrupt such binding allows modulation of these processes for clinical purposes. In the Examples set out herein, the inventors demonstrate the ability of antibodies of the invention to inhibit or disrupt PSGL-1 binding to selectins (such as P-selectin) or to chemokines (such as CCL27).

These ligands are known to be associated with the homing of inflammatory cells to sites of inflammation. The ability to inhibit or disrupt binding of PSGL-1 to these ligands may facilitate inhibition of the pathophysiological recruitment and transmigration of leukocytes (including eosinophils, neutrophils, and lymphocytes), such as that occurring into the tissue and airways of the lung in asthma and COPD. Thus, the results obtained to date support a scientific rationale for using the antibodies, methods of treatment or medical uses of the invention for the treatment of asthma or COPD.

The results also demonstrate the surprising effectiveness of the antibodies, methods and uses in such treatment.

In view of this surprising clinical effectiveness, and of the common role that the binding of PSGL-1 to its ligands plays in the initiation or propagation of damaging biological activities, particularly those associated with extravasation and influx of inflammatory cells, the skilled person will also appreciate that the antibodies, methods of treatment, and medical uses of the invention may be expected to have clinical applications beyond those directly demonstrated herein.

### Anti-Inflammatory Applications

The data provided in the Examples illustrate that the antibodies of the invention are suitable for use as anti-inflammatory agents. In the same manner, the methods of treatment and medical uses of the invention may be used in the prevention or treatment of inflammation, or of a disease, or condition associated with inflammation.

The antibodies, medical uses, or methods of treatment of the invention may be of use in the prevention or treatment of inflammatory conditions or diseases selected from the group consisting of: asthma; chronic obstructive pulmonary disease; allergic reactions; inflammatory bowel disease (e.g., Crohn's disease, ulcerative colitis, enteritis); arthritis (e.g., rheumatoid arthritis, osteoarthritis, psoriatic arthritis); graft rejection; graft versus host disease; psoriasis; dermatitis; nephritis; lupus erythematosus; scleroderma; rhinitis; anaphylaxis; diabetes; multiple sclerosis; atherosclerosis; and thyroiditis.

The antibodies, methods of treatment and medical uses of the invention are of particular use in the prevention or treatment of inflammatory diseases or conditions associated with the extravasation and accumulation of inflammatory cell types selected from the group consisting of: eosinophils; and neutrophils.

The antibodies of the invention may be used as an immunosuppressant agent. The skilled person will readily be able to identify clinical circumstances in which it may be wished to make therapeutic use of the immunosuppressive activity of the antibodies of the invention, or antibodies suitable for use in the methods or uses of the invention.

### Eosinophilic Conditions and Diseases

Eosinophils are a type of white blood cell that contribute to initiation and modulation of inflammation. Eosinophilic conditions occur when large numbers of eosinophils are recruited to specific sites within the body. This may particularly arise as a result of extravasation, with eosinophils cross blood vessel walls to enter inflamed tissue. The presence of increased number of eosinophils within tissues and cause them to become damaged.

As demonstrated in the Examples, the antibodies of the invention, and methods of treatment and medical uses of the invention, have proven to be surprisingly clinically effective in alleviating eosinophilic conditions. Accordingly, the use of the antibodies of the invention, and/or the methods of treatment and medical uses of the invention, for the prevention or treatment of eosinophilic conditions constitutes a particularly suitable embodiment of these various aspects of the invention.

Suitably, the eosinophilic condition or disease to be prevented or treated may be selected from the group consisting of: eosinophilic asthma; allergies; eosinophilic oesophagitis; eosinophilic dermatitis; contact dermatitis; acute myelogenous leukemia (AML), ascariasis; atopic dermatitis (eczema); bullous pemphigoid; cancer (such as Hodgkin lymphoma, leukemia, and certain myeloproliferative neoplasms); Churg-Strauss syndrome; drug allergy; eosinophilic cardiomyopathy; eosinophilic cellulitis (Wells' syndrome); eosinophilic colitis; eosinophilic enteritis; eosinophilic fasciitis; eosinophilic gastrointestinal diseases; eosinophilic granulomatosis with polyangiitis (EGPA); eosinophilic leukemia; eosinophilic myocarditis; hay fever (allergic rhinitis); Hodgkin's lymphoma (Hodgkin's disease); hypereosinophilic syndromes; idiopathic hypereosinophilic syndrome (HES); IgG4-Related Disease; inflammatory bowel disease (Crohn's disease, ulcerative colitis); lymphatic filariasis; neuromyelitis optica (NMO); ovarian cancer; parasitic infection; primary biliary cirrhosis; primary immunodeficiency; or trichinosis.

### Asthma

Asthma is a disease associated with long-term inflammation of the airways of the lungs. In asthma attacks constriction of the airways and bronchospasms lead to shortness of breath, tightness of the chest, and other symptoms such as coughing and wheezing. Attacks may be triggered by activities, or a range of environmental conditions, and asthma may include an allergic component. Asthma is believed to have been responsible for around 461,000 deaths worldwide in 2019.

As shown in the Examples, the inventors have demonstrated that the antibodies of the invention, and the methods of treatment and medical uses of the invention, may be used very effectively in the prevention or treatment of asthma. The antibodies, and methods of treatment or medical uses, of the invention may be of benefit in the prevention or treatment of moderate or severe asthma. In particular, they may be useful in the prevention or treatment of eosinophilic asthma.

As the Examples clearly demonstrate, treatment of asthma using the antibodies of the invention (in an example of a method of treatment or medical use of the invention), is able to bring about a statistically significant improvement in lung function. The methods of treatment and medical uses of the invention thus represent an effective asthma therapy. Indeed, the results show that the improvement provided by the agents and treatments of the invention is greater than that achieved using leading treatments known from the prior art, and so represents an important advance in the therapies available to asthma patients.

The ability of the antibodies identified by the inventors to inhibit or disrupt binding of PSGL-1 to its ligands indicates that these agents may suitably be used in asthma management and therapy to prevent the occurrence of asthma attacks, or to alleviate symptoms associated with ongoing attacks.

In particular, the methods of treatment or medical uses of the invention may be used for the prevention or treatment of asthma by attenuation of allergen-induced bronchioconstriction. The data disclosed in the Examples indicate that treatments of the invention are effective in reducing such constriction.

Suitably, asthma to be prevented or treated may be selected from the group consisting of: eosinophilic asthma; allergic asthma; and severe neutrophilic asthma.

Since the antibodies of the invention and/or methods of treatment and medical uses of the invention, are able to bring about a reduction in the extravasation of eosinophils that drives eosinophilic asthma, they may be of particular utility in the prevention or treatment of eosinophilic asthma.

A suitable embodiment of the ninth aspect of the invention, provides a method of preventing or treating asthma in a subject in need thereof, the method comprising providing a therapeutically effective amount of an anti-PSGL-1 antibody of the second aspect of the invention to the subject.

A suitable embodiment of the tenth aspect of the invention provides an anti-PSGL-1 antibody of the second aspect of the invention for use in the prevention or treatment of asthma.

### Neutrophilic Diseases and Disorders

Neutrophils are very abundant white blood cells that contribute to initiation and modulation of inflammation. Neutrophilic conditions occur when large numbers of neutrophils are recruited to specific sites within the body. This may particularly arise as a result of extravasation, with neutrophils cross blood vessel walls to enter inflamed tissue. The presence of increased number of neutrophils within tissues and cause them to become damaged.

As demonstrated in the Examples, the antibodies of the invention, and methods of treatment and medical uses of the invention, have proven to be surprisingly clinically effective in reducing the extravasation and accumulation of neutrophils. Accordingly, the antibodies, and methods of treatment and medical uses, of the invention offer promise in alleviating neutrophilic conditions. Accordingly, the use of the antibodies of the invention, and/or the methods of treatment and medical uses of the invention, for the prevention or treatment of neutrophilic conditions constitutes a particularly suitable embodiment of these various aspects of the invention.

Suitably, the neutrophilic condition or disease to be prevented or treated may be selected from the group consisting of: COPD; neutrophilic dermatoses (e.g., Sweet's syndrome, amicrobial pustulosis of the folds, erythema elevatum diutinum, amicrobial pustulosis of the scalp/leg, amicrobial subcorneal pustulosis and pyoderma gangrenosum); neutrophilic asthma; rheumatoid arthritis (RA); cystic fibrosis (CF); neutrophilic vasculitis (e.g., antineutrophil cytoplasmic antibody (ANCA)-associated vasculitis); gout; IBD (e.g., Crohn's disease and ulcerative colitis); neutrophilic meningitis; behçet's disease; PAPA syndrome; hidradenitis suppurativa; acne due to EGFRIs; PASH syndrome; PAPASH syndrome, neutrophilic panniculitis; aseptic abscess syndrome; systemic inflammatory response syndrome (SIRS); severe septicaemia; cryopyrin-associated periodic syndromes (CAPS); neutrophilic otitis externa; allergies; Chronic neutrophilic leukemia (CNL); hay fever (allergic rhinitis); Severe Acute Respiratory Syndrome (SARS); Middle East Respiratory Syndrome (MERS); SARS-COV; SARS-COV-2; and necrotizing enterocolitis (NEC).

### Chronic Obstructive Pulmonary Disease

Chronic obstructive pulmonary disease (COPD) is a progressive lung disease, associated with coughing and breathing difficulties. Forms of COPD may include emphysema and chronic bronchitis. Prolonged inflammation in the lungs, typically mediated by eosinophil, neutrophil or macrophage inflammatory cells, can cause remodelling and narrowing of the airways that contribute to reduced lung function. There are currently no cures available for COPD, but the disease can be prevented or managed using suitable treatments. COPD accounted for around 3.2 million deaths worldwide in 2019.

Eosinophil extravasation into the tissue of the lungs contributes to the establishment of elevated numbers of local eosinophils in certain population of subjects with COPD. It is known that the number of inflammatory cells present in the lungs correlates well with the severity of disease.

In the clinical trial results reported in Example 8, the inventors have demonstrated that treatment with the antibodies of the invention (exemplified by SelK2) reduces the number of extravasated eosinophils in lungs of patients with COPD. The same results have also shown that treatment of COPD patients with antibodies of the invention brings about a reduction in number of neutrophils present.

The skilled person will appreciate that these results, involving a reduction in the numbers of two types of white blood cells that are considered to play an essential role in driving the inflammation underlying COPD, illustrate that the antibodies of the invention (and hence methods of treatment and medical uses of the invention) are able to provide effective therapies for the treatment of COPD.

The reduction in neutrophil numbers may occur as a result of reduced adhesion, rolling and extravasation of these cells, due to the inhibition or disruption of PSGL-1-mediated binding. This is consistent with the reduction in adhesion and rolling of neutrophils observed in Examples 4 and 6.

In view of the above, it will be recognised that the use of the antibodies of the invention, or of the methods of treatment or medical uses of the invention, in the prevention or treatment of COPD represent particularly suitable embodiments of these various aspects of the invention.

The inventors have also observed that COPD patients receiving treatment in accordance with the invention exhibit a reduction in the number of epithelial cells present in sputum (when compared to controls).

A suitable embodiment of the ninth aspect of the invention, provides a method of preventing or treating COPD in a subject in need thereof, the method comprising providing a therapeutically effective amount of an anti-PSGL-1 antibody of the second aspect of the invention to the subject.

A suitable embodiment of the tenth aspect of the invention provides an anti-PSGL-1 antibody of the second aspect of the invention for use in the prevention or treatment of COPD.

### Respiratory Diseases or Conditions

Given the successes observed using antibodies and methods of treatment of the invention in both asthma and COPD, it will be recognised that the invention is well suited to use in the context of prevention or treatment of respiratory diseases or conditions. In particular, the antibodies or methods of treatment or medical uses of the invention may be employed in the prevention or treatment of respiratory diseases or conditions associated with inflammation. The antibodies or methods of treatment or medical uses of the invention may be employed in the prevention or treatment of respiratory diseases or conditions associated with the activity of eosinophils or neutrophils.

By way of example, the antibodies of the invention, or the methods of treatment or medical uses of the invention, may be employed in the prevention or treatment of a respiratory disease or condition selected from the group consisting of: eosinophilic pneumonia (whether chronic or acute); simple pulmonary eosinophilia (Loeffler syndrome); allergic bronchopulmonary aspergillosis; eosinophilic granulomatosis with polyangiitis; and iatrogenic diseases, such as eosinophilia caused by exposure to sulphonamides.

### Sickle Cell Disease (SCD)

SCD is a genetic blood disorder characterized by the presence of abnormal haemoglobin known as haemoglobin S (HbS). This abnormal haemoglobin causes red blood cells to take on a characteristic sickle or crescent shape, leading to a range of health complications, including pain crises, anemia, and organ damage.

Key pathological features of SCD are vaso-occlusion, where sickle-shaped red blood cells clump together and block small blood vessels, and chronic inflammation, associated with an increase in eosinophil counts or activation of eosinophils. These processes lead to symptoms of SCD such as pain crises and tissue damage.

The formation of complexes between PSGL-1 and P-selectin plays a major role in both of these processes. As noted above, binding of PSGL-1 to its ligands plays an important role in the extravasation and influx of inflammatory cells associated with inflammation. Furthermore, upregulation of P-selectin in endothelial cells and platelets contributes to the cell-to-cell interactions involved in the pathogenesis of vaso-occlusion and pain crisis in SCD. In view of the antibodies of the invention's demonstrated ability to inhibit formation of complexes between PSGL-1 and P-selectin and also to disrupt such complexes that have already formed, it will be recognised that the invention is well suited to use in the context of prevention or treatment of SCD or its symptoms. In particular, the antibodies or methods of treatment or medical uses of the invention may be employed in the prevention or treatment of SCD or symptoms of SCD.

A suitable embodiment of the ninth aspect of the invention provides a method of preventing or treating SCD in a subject in need thereof, the method comprising providing a therapeutically effective amount of an anti-PSGL-1 antibody of the second aspect of the invention to the subject.

A suitable embodiment of the tenth aspect of the invention provides an anti-PSGL-1 antibody of the second aspect of the invention for use in the prevention or treatment of SCD.

In the context of the present disclosure, and particularly with reference to the ninth or tenth aspects of the invention, preventing or treating SCD should be taken as encompassing treating or preventing any of the processes, such as vaso-occlusion or chronic inflammation, that give rise to the damaging effects of SCD. Suitably, preventing or treating SCD should be taken as encompassing preventing or treating symptoms associated with SCD. In particular, preventing or treating SCD should be taken as encompassing preventing or treating pain crisis associated with SCD.

The ability of the antibodies of the invention (or of antibodies suitable for use in the methods or uses of the invention) to inhibit formation of complexes between PSGL-1 and P-selectin is indicative of their utility in preventing SCD, or symptoms of SCD such as pain crisis, before the disease or symptoms occur. The ability of the antibodies of the invention (or of antibodies suitable for use in the methods or uses of the invention) to disrupt complexes between PSGL-1 and P-selectin that have already formed, is indicative of their utility in treating SCD, or symptoms of SCD such as pain crisis, once the disease or symptom is already underway. Given the distress caused to patients by SCD pain crisis, it will be recognised that agents able to treat patients undergoing such crises, and thereby provide relief from the pain, are highly desirable.

### Treatment Regimens

Methods of treatment or medical uses in accordance with the invention may employ treatment regimens selected with a view to factors including:
the nature of the condition or disease;
the properties of the antibody to be used;
whether prevention or treatment is required.

Suitably treatment regimens may also take into account factors such as the age or weight of a subject receiving treatment, and the severity of the subject's disease.

Pharmacokinetic properties of the antibody used in a method of treatment or medical use of the invention may inform the treatment regimen. By way of guidance, Example 7 demonstrates that the exemplary antibody of the invention SelK2 has a half-life and pharmacodynamic profile that indicate that effective concentrations of the antibody within the serum may be maintained by monthly administration.

As considered elsewhere in this specification, applications in which it is wished to prevent a condition or disease may require the establishment and maintenance of a concentration of antibody in the serum sufficient to inhibit binding of PSGL-1 and its ligands, while applications in which it is wished to treat a condition or disease may require the establishment and maintenance of a concentration of antibody in the serum sufficient to disrupt binding of PSGL-1 and its ligands.

Merely by way of example, incidences of treatment may be repeated on multiple occasions per day, or may be repeated daily. Incidences of treatment may be repeated every week, every 2 weeks, every 3 weeks, every 4 weeks, every month, every 5 weeks, every 6 weeks, or at longer intervals.

The Examples demonstrate the effectiveness of treatment regimens in which an antibody of the invention is administered (for example at a dose of 7.5 mg/kg bodyweight) with a three week period between incidences of administration. Such regimens may be of benefit in the treatment of patients with respiratory disorders, such as asthma or COPD.

A suitable embodiment of a dosing regimen that may be employed in the methods of treatment or medical uses of the invention involves a loading dose approach. The first two incidences of treatment may be separated by two weeks, with further incidences of treatment occurring approximately every four weeks.

### Therapeutically Effective Amounts of Antibodies

The methods of treatment of the invention involve the provision of therapeutically effective amounts of antibodies of the invention (or of antibodies suitable for use in the methods or uses of the invention). Such therapeutically effective amounts of the requisite antibodies may be provided in a single incidence of treatment, or may be accumulated over the course of a number of incidences of treatment.

A therapeutically effective amount in respect of an antibody of the invention, or an antibody suitable for use in the methods of treatment or medical uses of the invention, may be defined in terms of the amount of the antibody to be administered per kilogram of total body weight of the patient receiving the antibody.

The Examples demonstrate the effectiveness of treatment regimens in which an antibody of the invention is administered at a dose of 7.5 mg/kg body weight (for example with a three week period between incidences of administration). Such regimens may be of benefit in the treatment of patients with respiratory disorders, such as asthma or COPD.

For example, an antibody may be administered to a patient in a therapeutically effective amount of approximately 1 mg/kg, approximately 1.5 mg/kg, approximately 2 mg/kg, approximately 2.5 mg/kg, approximately 3 mg/kg, approximately 3.5 mg/kg, approximately 4 mg/kg, approximately 4.5 mg/kg, approximately 5 mg/kg, approximately 5.5 mg/kg, approximately 6 mg/kg, approximately 6.5 mg/kg, approximately 7 mg/kg, approximately 7.5 mg/kg, approximately 8 mg/kg, approximately 8.5 mg/kg, approximately 9 mg/kg, approximately 9.5 mg/kg, approximately 10 mg/kg, approximately 10.5 mg/kg, approximately 11 mg/kg, approximately 12 mg/kg, approximately 13 mg/kg, approximately 14 mg/kg, approximately 15 mg/kg, approximately 20 mg/kg, approximately 25 mg/kg, or approximately 30 mg/kg.

In a suitable embodiment, a therapeutically effective amount of an antibody is between approximately 10 mg and approximately 5000 mg. For example, in a suitable embodiment, a therapeutically effective amount of an antibody is between approximately 100 mg and approximately 1500 mg, for example between approximately 200 mg and approximately 1000 mg, or between approximately 450 mg and approximately 750 mg. Suitably, a therapeutically effective amount of an antibody of the invention is approximately 600 mg.

### Routes of Administration

When practicing the methods of treatment or medical uses of the invention, antibodies, such as the antibodies of the invention may be provided to a subject by any suitable route of administration.

Since the antibodies may be useful in preventing extravasation of leukocytes from the blood stream, it will be recognised that the antibodies may be administered intravenously in a suitable embodiment of the invention. Such intravenous administration may be by means of intravenous injection, or by means of intravenous infusion, as described further in the Examples.

Infusion in accordance with this embodiment of the invention may take place over any suitable period of time. Merely by way of example, infusion may be for a period of 30 minutes (or approximately 30 minutes).

Suitably, antibodies may be provided by intravenous infusion. This route of administration has been shown to be effective in the prevention or treatment of asthma and COPD.

Injection is a well-established route for the administration of therapeutic antibodies, and so represents a suitable embodiment in respect of the provision when practicing methods of treatment or medical uses of the present invention.

Suitably, antibodies are provided to a subject requiring prevention or treatment by means of injection. Suitably, antibodies are provided to a subject requiring prevention or treatment by means of intravenous injection. Suitably, antibodies are provided to a subject requiring prevention or treatment by means of intramuscular injection. Suitably, antibodies are provided to a subject requiring prevention or treatment by means of subcutaneous injection.

### Pharmaceutical Compositions

Pharmaceutical compositions of the invention comprise an antibody of the invention, or an antibody suitable for use in the methods of treatment or medical uses of the invention. In addition to the antibody, a pharmaceutical composition of the invention comprises a suitable pharmaceutically acceptable excipient.

Pharmaceutical compositions may be formulated with reference to their intended route of administration. Once a chosen route of administration has been selected, examples of suitable formulations may be readily identified by the skilled person.

Suitably, a pharmaceutical composition of the invention is suitable for administration by intravenous infusion. An exemplary pharmaceutical composition of the invention suitable for administration by intravenous infusion is described in the Examples, and this composition has been shown to be effective in the prevention or treatment of asthma and COPD.

Suitably, a pharmaceutical composition of the invention is suitable for injection. Suitably, a pharmaceutical composition of the invention is suitable for intravenous injection. Suitably, a pharmaceutical composition of the invention is suitable for intramuscular injection. Suitably, a pharmaceutical composition of the invention is suitable for subcutaneous injection.

In the case of a pharmaceutical composition of the invention for administration by injection, a suitable excipient may comprise a diluent.

A pharmaceutical composition of the invention may suitably comprise an antibody of the invention, or an antibody suitable for use in the methods of treatment or medical uses of the invention, and a phosphate buffered saline solution. In a suitable embodiment of such a composition the phosphate buffered saline comprises sodium phosphate at a concentration of approximately 25 mM and sodium chloride at a concentration of approximately 190 mM.

In a suitable embodiment, a pharmaceutical composition of the invention comprises an excipient such as polysorbate 80. By way of example, a pharmaceutical composition of the invention may comprise polysorbate 80 at a concentration of approximately 0.02% (w/w). Examples of other excipients that may be employed in the pharmaceutical compositions of the invention are described elsewhere in the present disclosure.

A pharmaceutical composition of the invention may have a substantially neutral pH. In such an embodiment, a pharmaceutical composition of the invention may have a pH of 7.0±0.5.

Suitably, a pharmaceutical composition of the invention may comprise an antibody of the invention, or an antibody suitable for use in the methods of treatment or medical uses of the invention, at a concentration of between approximately 1 mg/mL and approximately 1000 mg/mL. For example, a pharmaceutical composition of the invention may comprise an antibody of the invention, or an antibody suitable for use in the methods of treatment or medical uses of the invention, at a concentration of between approximately 2 mg/ml and approximately 90 mg/mL, of between approximately 3 mg/mL and approximately 80 mg/mL, of between approximately 4 mg/mL and approximately 70 mg/mL, of between approximately 5 mg/mL and approximately 60 mg/mL, of between approximately 6 mg/mL and approximately 50 mg/mL, of between approximately 7 mg/mL and approximately 40 mg/mL, of between approximately 8 mg/mL and approximately 30 mg/mL, or of between approximately 9 mg/mL and approximately 20 mg/mL.

Suitably, a pharmaceutical composition of the invention may comprise an antibody at a concentration of approximately 10 mg/mL

Suitably, a pharmaceutical composition of the invention may be provided in the form of a dosage unit of between approximately 1 mL and approximately 100 mL. For example, a pharmaceutical composition of the invention may be provided in the form of a dosage unit of between approximately 2 mL and approximately 90 mL, of between approximately 3 mL and approximately 80 mL, of between approximately 4 mL and approximately 70 mL, of between approximately 5 mL and approximately 60 mL, of between approximately 6 mL and approximately 50 mL, of between approximately 7 mL and approximately 40 mL, of between approximately 8 mL and approximately 30 mL, or of between approximately 9 mL and approximately 20 mL.

Suitably, a pharmaceutical composition of the invention may be provided in the form of a dosage unit of approximately 10 mL.

Suitably, a pharmaceutical composition of the invention is suitable for administration by intravenous infusion. A pharmaceutical composition of the invention administered by intravenous infusion has been shown to be effective in the prevention or treatment of asthma and COPD.

Suitably, a pharmaceutical composition of the invention is suitable for injection. Suitably, a pharmaceutical composition of the invention is suitable for intravenous injection. Suitably, a pharmaceutical composition of the invention is suitable for intramuscular injection. Suitably, a pharmaceutical composition of the invention is suitable for subcutaneous injection.

### Polynucleotide Sequences, Expression Vectors, and Cells of the Invention

The invention provides polynucleotide sequences (which may be referred to, for brevity, as “polynucleotides”) encoding antibodies of the invention, or antibodies suitable for use in the methods of treatment or medical uses of the invention, or encoding antigen binding fragments of such antibodies. The antibodies may be in accordance with any of the aspects or embodiments described herein.

A polynucleotide sequence of the invention may encode an immunoglobulin chain of a suitable antibody. Suitably a polynucleotide sequence encodes an immunoglobulin chain of an antibody of the invention. References to the immunoglobulin chains encoded by the polynucleotides of the invention should be taken as encompassing the variable region of an appropriate antibody, a full-length immunoglobulin chain of an appropriate antibody, or antigen binding fragment (e.g.an scFv fragment) of an appropriate antibody.

According to a fourth aspect of the invention, there is provided a polynucleotide sequence encoding an immunoglobulin chain of a humanised anti-PSGL-1 antibody comprising at least one CDR of the antibody heavy chain set out in SEQ ID NO: 1 and at least one CDR of the antibody light chain set out in SEQ ID NO: 2.

According to a fifth aspect of the invention, there is provided a polynucleotide sequence encoding a humanised anti-PSGL-1 antibody, or antigen binding fragment thereof, comprising an immunoglobulin chain comprising at least one CDR of the antibody heavy chain set out in SEQ ID NO: 1 and an immunoglobulin chain comprising at least one CDR of the antibody light chain set out in SEQ ID NO: 2.

By way of example, a polynucleotide in accordance with either of these aspects of the invention may encode an immunoglobulin heavy chain of a humanised anti-PSGL-1 antibody comprising at least one CDR of the antibody heavy chain set out in SEQ ID NO: 1. Such a polynucleotide may encode an immunoglobulin heavy chain of a humanised anti-PSGL-1 antibody comprising at least two CDRs of the antibody heavy chain set out in SEQ ID NO: 1. Such a polynucleotide may encode an immunoglobulin heavy chain of a humanised anti-PSGL-1 antibody comprising all three CDRs of the antibody heavy chain set out in SEQ ID NO: 1.

Alternatively, or additionally, a polynucleotide in accordance with these aspects of the invention may encode an immunoglobulin light chain of a humanised anti-PSGL-1 antibody comprising at least one CDR of the antibody light chain set out in SEQ ID NO: 2. Such a polynucleotide may encode an immunoglobulin light chain of a humanised anti-PSGL-1 antibody comprising at least two CDRs of the antibody light chain set out in SEQ ID NO: 2. Such a polynucleotide may encode an immunoglobulin light chain of a humanised anti-PSGL-1 antibody comprising all three CDRs of the antibody light chain set out in SEQ ID NO: 2.

A suitable polynucleotide of the invention may encode both a heavy chain that comprises at least one CDR of the antibody heavy chain set out in SEQ ID NO: 1, and a light chain that comprises at least one CDR of the antibody light chain set out in SEQ ID NO: 2. A suitable polynucleotide of the invention may encode both a heavy chain that comprises at least two CDRs of the antibody heavy chain set out in SEQ ID NO: 1, and a light chain that comprises at least two CDRs of the antibody light chain set out in SEQ ID NO: 2. A suitable polynucleotide of the invention may encode both a heavy chain that comprises all three CDRs of the antibody heavy chain set out in SEQ ID NO: 1, and a light chain that comprises all three CDRs of the antibody light chain set out in SEQ ID NO: 2.

Suitable polynucleotide sequences of the invention may further be defined with reference to the variable sequences of the antibodies that they encode.

For example, the eleventh aspect of the invention provides a polynucleotide sequence encoding the heavy chain variable sequence set out in SEQ ID NO: 26. Suitably, such a polynucleotide of the eleventh aspect of the invention may encode the heavy chain sequence set out in SEQ ID NO: 1.

Furthermore, the twelfth aspect of the invention provides a polynucleotide sequence encoding the light chain variable sequence set out in SEQ ID NO: 29. Suitably, such a polynucleotide of the twelfth aspect of the invention may encode the light chain sequence set out in SEQ ID NO: 2.

By way of example of a polynucleotide in accordance with the invention, there is provided the polynucleotide set out in SEQ ID NO: 42 that encodes a polypeptide comprising the heavy chain sequence of SEQ ID NO: 1 and a 19 amino acid leader peptide at the N-terminal. The invention also provides the polynucleotide set out in SEQ ID NO: 43 that encodes a polypeptide comprising the light chain sequence of SEQ ID NO: 2 and a 19 amino acid leader peptide at the N-terminal.

In a suitable embodiment, a nucleic acid in accordance with this aspect of the invention comprises the sequence set out in SEQ ID NO: 42 encoding an immunoglobulin heavy chain and the sequence set out in SEQ ID NO: 43 encoding an immunoglobulin light chain. In a suitable embodiment, a polynucleotide of the invention shares at least 90% identity with the sequence set out in SEQ ID NO: 42 or with the sequence set out in SEQ ID NO: 43. In a suitable embodiment, the polynucleotide of the invention shares at least 91%, at least 92%, least 93%, at least 94%, least 95%, at least 96%, least 97%, at least 98%, or at least 99% identity with the light chain sequence set out in SEQ ID NO: 15 and the heavy chain sequence set out in SEQ ID NO: 16. Such calculations of identity may discount the parts of the polynucleotides encoding the leader peptides.

Suitable polynucleotides may include one or more of: polymers of deoxyribonucleic acids (DNAs), polymers of ribonucleic acids (RNAs), analogues of these DNAs or RNAs generated using nucleotide analogues, and derivatives, fragments and homologs thereof.

A polynucleotide in accordance with the invention may be optimised for expression in mammalian cells. A suitable polynucleotide may be optimised for expression in a mammalian cell line, such as Chinese hamster ovary (CHO) cells.

Polynucleotides of the invention may be provided as part of a larger nucleic acid molecule. Merely by way of example, a polynucleotide of the invention may be provided as part of an expression construct comprising a first polynucleotide sequence of the invention encoding a first immunoglobulin chain comprising at least one CDR of the antibody heavy chain set out in SEQ ID NO: 1 and a second polynucleotide sequence of the invention encoding a second immunoglobulin chain comprising at least one CDR of the antibody light chain set out in SEQ ID NO: 2. Such an expression construct may further comprise one or more of: a promoter; and an internal ribosome entry site located between the first and second polynucleotide sequences.

The present invention provides expression vectors which comprise a polynucleotide of the invention and host cells comprising an expression vector of the present invention. Host cells comprising an expression vector of the present invention may be used, e.g., in methods for producing an antibody of the present invention by recombinant expression.

The term “expression vector,” as used herein, refers to a nucleic acid molecule comprising a polynucleotide of the invention, linear or circular, comprising one or more expression units. In a suitable embodiment, an expression vector comprising a polynucleotide in accordance with the invention may be a DHFR expression vector.

The invention also provides a cell comprising a polynucleotide sequence in accordance with the invention. Such a cell may comprise a polynucleotide sequence in accordance with the fourth, fifth, eleventh or twelfth aspects of the invention.

For example, the sixth aspect of the invention provides a cell comprising a polynucleotide sequence encoding an immunoglobulin chain of a humanised anti-PSGL-1 antibody, or antigen binding fragment thereof, comprising at least one CDR of the antibody heavy chain set out in SEQ ID NO: 1 and at least one CDR of the antibody light chain set out in SEQ ID NO: 2.

The seventh aspect of the invention provides a cell comprising a polynucleotide sequence encoding a humanised anti-PSGL-1 antibody, or antigen binding fragment thereof, comprising an immunoglobulin chain comprising at least one CDR of the antibody heavy chain set out in SEQ ID NO: 1 and an immunoglobulin chain comprising at least one CDR of the antibody light chain set out in SEQ ID NO: 2.

Furthermore, as set out above, the thirteenth aspect of the invention provides a cell comprising a polynucleotide sequence encoding the heavy chain sequence set out in SEQ ID NO: 1 and a polynucleotide sequence encoding the light chain sequence set out in SEQ ID NO: 2.

In any of these aspects of the invention, polynucleotide sequence, or sequences, may be provided in the form of an expression vector.

A cell of the invention may be a “host cell” which can support the replication or expression of an expression vector. Cells of the invention, such as host cells, may be prokaryotic cells, such as E. coli, or may be eukaryotic cells. Suitably, cells of the invention, such as host cells, are cells of a mammalian cell line. Suitably, cells of the invention, such as host cells, are CHO cells.

A polynucleotide in accordance with the invention may be an isolated polynucleotide.

### Method of Manufacturing

The eighth aspect of the invention provides a method of manufacturing a humanised anti-PSGL-1 antibody comprising at least one CDR of the antibody heavy chain set out in SEQ ID NO: 1 and at least one CDR of the antibody light chain set out in SEQ ID NO: 2. The method comprises:
expressing a first polynucleotide sequence encoding an immunoglobulin chain of a humanised anti-PSGL-1 antibody comprising at least one CDR of the antibody heavy chain set out in SEQ ID NO: 1; and
expressing a second polynucleotide sequence encoding an immunoglobulin chain of a humanised anti-PSGL-1 antibody comprising at least one CDR of the antibody light chain set out in SEQ ID NO: 2.

Suitably expression of the first and second polynucleotides may take place in the same cells. Alternatively, expression of the first and second polynucleotides may be in first and second populations of cells. Cells used in accordance with this aspect of the invention may be as considered for the cells of the invention and host cells above.

The first and second polynucleotide sequences employed in a method of the invention may be introduced into the cells by which they are expressed. Such a step of introducing the first and second polynucleotide sequences may be feature of such a method. The first and second polynucleotide sequences may be incorporated into one or more expression vectors.

A method in accordance with this aspect of the invention may further comprise a step of purifying the antibody produced.

### EXAMPLES

### Example 1. Humanization of SelK2

Humanization of heavy and light chains was performed by CDR grafting and utilised germline framework acceptor regions to reduce potential immunogenicity of the antibody. An engineered IgG2 constant region was employed to reduce inherent antibody effector functions, such as Fc receptor binding and complement activation.

### Parental Antibody

The amino acid sequence of the parental murine antibody was determined by LC-MS-MS. The amino acid sequence of the heavy chain variable region of the parental murine antibody is set out in SEQ ID NO: 21, and the amino acid sequence of the light chain variable region of the parental murine antibody is set out in SEQ ID NO: 22. For the avoidance of doubt, the parental antibody does not constitute a humanised anti-PSGL-1 antibody of the invention.

### Design, Production, and Testing of Several Humanized Forms

Human germline sequences were chosen as acceptor sequences to minimize potential immunogenicity. For the heavy chain, human germline sequence VH3 1-3 3-30.5 (SEQ ID NO: 23) was chosen and the framework 4 sequence (highlighted in green) was from human germline heavy joining sequence JH4a. For the light chain, human germline sequence IGKV2-40*01 (SEQ ID NO: 24) was chosen; and the framework 4 sequence (FGQGTRLEIKR) was from human germline kappa joining sequence JK 5.

Humanized versions of the heavy (SEQ ID NO: 25) and light (SEQ ID NO: 29) chains were designed by grafting the CDRs of the parental antibody into the human acceptor framework sequences without further modifications or back-mutations (straight Graft versions).

Humanized versions were also designed with 1, 2, or 5 back-mutations (B1, B2 or B5) for the heavy chain (SEQ ID NO: 26, SEQ ID NO: 27, and SEQ ID NO: 28, respectively) and with 2 or 4 back-mutations (B2 or B4) for the light chain (SEQ ID NO: 30 and 31, respectively). The back-mutations were made to the original amino acid residues of the parental antibody, as described in Table 1.

TABLE 1
Back-mutations in the heavy and light chain human germline sequences

| Humanized | |
|---|---|
| version | Back-Mutation |
| Heavy chain - B1 | K residue at position 98 of SEQ ID NO: 25 to S |
| Heavy chain - B2 | K residue at position 98 of SEQ ID NO: 25 to S and S residue at |
| | position 75 of SEQ ID NO: 25 to P. |
| Heavy chain - B5 | K residue at position 98 of SEQ ID NO: 25 to S; S residue at position |
| | 75 of SEQ ID NO: 25 to P; R residue at position 16 of SEQ ID NO: 25 |
| | to G; L residue at position 18 of SEQ ID NO: 25 to R; and G residue |
| | at position 42 of SEQ ID NO: 25 to E. |
| Light chain - B2 | P residue at position 15 of SEQ ID NO: 29 to L and P residue at |
| | position 18 of SEQ ID NO: 29 to Q |
| Light chain - B4 | P residue at position 15 of SEQ ID NO: 29 to L; P residue at position |
| | 18 of SEQ ID NO: 29 to Q; Y residue at position 92 of SEQ ID NO: 29 |
| | to F; Q residue at position 105 of SEQ ID NO: 29 to A. |

The humanized versions were engineered as human IgG1/kappa antibodies and transiently expressed by the OMRF Antibody Expression Group. This was done by sub-cloning the light chain variable regions into an expression vector containing the human kappa constant region coding sequence; and the heavy chain variable regions into an expression vector containing the human IgG 1 constant region coding sequence. Both the light and heavy chain expression vectors were driven by CMV promoters. The heavy and light chain expression vectors for each antibody construct were then co-transfected into HEK 293A cells for transient expression. The resulting antibodies were purified from culture medium using protein-A chromatography. A full description of the methodology used is provided (Smith, K., et al., 2009).

Affinity testing of humanized versions of the parental antibody was carried out by SensiQ Technologies, Oklahoma City, OK, using surface plasmon resonance (SPR). The testing was done on an SPR chip coated with streptavidin and loaded with biotinylated-GSP-6, a glycosulfopeptide matching the N-terminal region of human PSGL-1. An initial screen of the humanized versions was carried out to identify the best candidate for further development.

Combinations of light and heavy chains tested were: LG-HG (SEQ ID NOs: 25+29); LB2-HG (SEQ ID NOs: 25+30); LG-HB1 (SEQ ID NOs: 26+29; LG-HB2 (SEQ ID NOs: 27+29); and LG-HB5 (SEQ ID NOs 28+29).

Back-mutations present in the light chain disrupted binding affinity and thus, the light chain straight Graft version (LG), wherein CDRs of the parental antibody into the human acceptor framework sequences without further modifications or back-mutations, provided minimum disruption to antibody binding and functionality. Surprisingly, back-mutations in the heavy chain improved binding affinity.

Thus, based on the affinity testing results, the HB1 version (i.e. containing one back-mutation) of the heavy chain (SEQ ID NO: 26) and the LG Graft version (i.e. containing no back-mutations) of the light chain (SEQ ID NO: 29) were chosen for the SelK2 antibody in order to provide enhanced antibody binging affinity and functionality and reduce any potential immunogenicity. A DHFR-based expression vector encoding SelK2 was then developed in order to create a CHO-based production cell line expressing SelK2.

For the final SelK2 antibody, the heavy chain version (BI) was re-engineered into a human IgG2 constant region to eliminate potential antibody effector functions. IgG2 isotypes have been shown to have minimal Fc receptor and complement component C1q binding capacity. A single amino acid change was introduced in the constant region to further eliminate C1q binding (SEQ ID NO: 1), K residue to an A residue at position 333 in SEQ ID NO: 1. This design was enabled by DNA synthesis at GenScript [4], sub-cloned at the OMRF Antibody Expression Group, and sequence-verified by both vendors. The light chain version (Graft) was maintained as a human kappa construct (SEQ ID NO:2). The SelK2 antibody was transiently expressed and purified by the OMRF Antibody Expression Group.

### Example 2. Characterisation of SelK2

As shown in Table 2, the monosaccharide composition of SelK2 is as follows:

TABLE 2
Monosaccharide Composition of SelK2

| | Moles of monosaccharide per mole of protein, normalized | | | | | |
|---|---|---|---|---|---|---|
| | to 6 moles of Mannose. | | | | | |
| | N-Acetyl | N- Acetyl | | | | |
| Sample | Glucosamine | Galactosamine | Galactose | Mannose | Glucose | Fucose |
| 15-0783- | 3.9 | <LOQ | Not | 4.8 | Not | 1.6 |
| RS | | | Detected | | Detected | |
| 18-1730- | 6.1 | <LOQ | Not | 4.8 | 0.61 | 2.6 |
| RS | | | Detected | | | |
| 19-2829- | 5.4 | <LOQ | <LOQ | 4.6 | 0.37 | 1.0 |
| RS | | | | | | |

### Sialic Acid Analysis

The sialic acid content, both N-glycolneuraminic acid (NGNA) and N-acetylneuraminic acid (NANA), was determined after the release of sialic acid by mild acid hydrolysis. After reaction with o-phenylenediamine the derivatized sialic acid samples were separated by reverse phase HPLC with fluorescence detection. Quantification was performed against an external calibration curve. This method can separate and quantify both NGNA and NANA. NGNA (N-glycolyl neuraminic acid) was not detected. The levels of NANA (N-acetyl neuraminic acid) detected are below the LOQ of the assay.

From these results it is concluded that the stoichiometry for each reference standard is below the LOQ of 0.1 moles of sialic acid per mole of protein. This work was performed by Charles River Laboratories.

### Example 3. Affinity of the Parental Antibody and SelK2

SelK2 and the parental antibody were immobilized and their interaction with SelSP1 (PSGL-1 sulfopeptide) was characterized using SensíQ Pioneer.

### Materials and Methods

SensíQ Pioneer (S/N 13110154) was used for all SPR measurements at a controlled temperature of 25° C. SelK2 (Lot #14-2189) was dispensed into three 1 mL aliquots in the biosafety cabinet. Protein G was purchased from BioVision (Cat. #6510). Running buffer for Pioneer contained 10 mM HEPES pH 7.4, 150 mM NaCl, 3.4 mM EDTA and 0.005% Tween-20. All other reagents were purchased from Sigma Aldrich.

A new COOH2 biosensor was installed and conditioned according to CH0012 protocol. Protein G was immobilized via amine coupling. A mixture containing 0.1M EDC and 0.025M NHS was injected for 4 minutes over flow channel 1 (FC1) and FC2. 50 μg/mL Protein G in 10 mM Sodium acetate buffer pH 4.5 was injected for 15 minutes over FC1 and FC2. 1M Ethanolamine HCl solution pH 8.0 was injected for 5 minutes over FC1 and FC2. Approximately 1,145 response units (RU) of Protein G was coupled on FC1 and FC2 of the sensor chip. 50 nM SelK2 in system buffer was injected to capture approximately 2,000 RU SelK2 on FC1 and 1,000 RU on FC2. A dilution series of SelSP1 was prepared in system buffer: 50, 25, 12.5, 6.25, and 3.125 nM. Each sample was injected for 3 min at a flow rate of 40 μL/min. Dissociation was monitored after each injection for 4 min at the same flow rate. Samples were tested in duplicate from low concentration to high.

Kinetic model fitting was performed using Qdat analysis software (SensiQ Technologies, Inc. Version 2.5.1.56). A simple 1:1 kinetic model was fit to the data to determine best fit values for association and dissociation rate constants, ka and kd. A local Rmax was fit for each curve to account for incomplete dissociation of the SelSP1 after some of the analyte injections. Global analysis of the binding responses revealed good agreement in kinetics and affinity between the 2,000 (high) RU and 1,000 (low) RU SelK2 surfaces. Table 2 shows the kinetic rate and affinity constants for this lot of SelK2.

### Results

TABLE 3
Kinetic rate constants of the parental
and SelK2 antibody binding SelSP1

| | ka (M⁻¹s⁻¹) | kd (s⁻¹) | KD (nM) |
|---|---|---|---|
| Parental antibody | 3.03 ± 0.01e5 | 4.97 ± 0.02e−3 | 16.38 ± 0.04 nM |
| SelK2 surface | 5.08 ± 0.02e5 | 2.179 ± 0.005e−3 | 4.29 ± 0.02 nM |
| High density | | | |
| SelK2 surface | 5.44 ± 0.02e5 | 2.322 ± 0.005e−3 | 4.27 ± 0.02 nM |
| Low density | | | |

As shown in FIG. 2 and Table 3, the parental antibody binds SelSP1 with an equilibrium dissociation constant (KD) of 16.4 nM. As shown in FIGS. 3, FIG. 4, and Table 4, the SelK2 antibody binds SelSP1 with an equilibrium dissociation constant (KD) of 4.28 nM. This result was consistent when tested at two surface densities of the antibody and using two replicates for all SelSP1 concentrations. Thus, surprisingly the SelK2 antibody has improved binding affinity to SelSP1 in comparison to the parental antibody.

### Example 4. SelK2 can Functionally Block Neutrophil Rolling on P-Selectin

### In Vitro Rolling Assay

The ability of SelK2 to functionally block PSGL-1 was tested by assessing its ability to block neutrophil rolling on P-selectin. In this assay, human neutrophils roll and tether to P-selectin coated on a plate at a density similar to that found on activated endothelial cells under flow conditions that simulate sheer stress in blood vessels.

### Materials and Methods

P-selectin purified from platelet membranes (mP-selectin) was prepared as described (Ushiyama et al., 1993). The parental antibody was purchased from Millipore (catalog #MAB4092). Human neutrophils were isolated from healthy donors as described (Zimmerman et al., 1985). A 30 μl drop of human mP-selectin (1 μg/ml) was placed in a demarcated area on a 35-mm culture plate (Corning) and incubated at 4° C. overnight. The area was washed twice with HBSS and then blocked with HBSS containing 1% human serum albumin at room temperature for 2 hours. Human neutrophils (4.5×10⁵/ml in HBSS containing 0.5% human serum albumin) were incubated with buffer alone (control) or SelK2 at concentrations of 1 μg/mL, 0.5 μg/mL, 0.25 μg/mL, 0.125 μg/mL, 0.06 μg/mL, 0.03 μg/mL, or 0.015 μg/mL for 5 minutes before the rolling experiments started. The cells were then perfused over the adsorbed mP-selectin in a parallel-plate flow chamber under shear stress at 1.0 dyn/cm2 (Ramachandran et al., 1999). After 5 minutes, dynamic images of several fields of view were captured and digitalized by a HAMAMATSU ORCA-Flash2.8 CCD digital camera. The accumulated number of bound and slow-rolling neutrophils from each run (SelK2 concentration) was measured in 5 to 6 different fields by image analysis software Element (Nikon) to calculate the average cell number and standard deviation.

TABLE 4
Neutrophil inhibition by SelK2.

| SelK2 (ug/mL) | Control (0) | 1 | 0.5 | 0.25 | 0.125 | 0.06 | 0.03 | 0.015 |
|---|---|---|---|---|---|---|---|---|
| Neutrophils/field | 66.5 | 1.25 | 1 | 8 | 37.5 | 65.25 | 69.75 | 68.25 |
| (average) | | | | | | | | |
| Standard | 6.14 | 0.96 | 0.82 | 4.97 | 8.10 | 9.88 | 9.07 | 15.31 |
| deviation | | | | | | | | |

SelK2 inhibited neutrophil rolling in this assay in a dose dependent manner over a concentration range of 0.125 μg/mL to 1 μg/mL, achieving near complete inhibition at 0.5 μg/mL, as shown in FIG. 5 and Table 4. These results demonstrate the ability of the SelK2 antibody to block PSGL-1 and inhibit neutrophil binding to P-selectin and rolling under shear stress.

### Example 5. SelK2 can Functionally Block and Inhibit Chemokine CCL27 Binding and Interacting with PSGL-1

The ability of SelK2 to block and inhibit chemokine CCL27 binding and interacting with PSGL-1 was assessed using an SPR-based assay. The assay utilizes a sulfopeptide (SelSP1) that is modelled after the N-terminus of human PSGL-1, and which interacts with CCL27 and is bound by K2 and SelK2.

### Materials and Methods

All reagents were purchased from Sigma Aldrich (St. Louis, MO) unless otherwise noted. SensíQ Pioneer was used for all SPR experiments. CCL27 (376-CT-025/CF) was supplied by Selexys from R&D Systems. SelSP1, K2, and SelK2 were also supplied by Selexys. Assay buffer contained 10 mM HEPES PH 7.4, 150 mM NaCl, 2 mM CaCl₂ and 0.01% (w/w) Tween-20. Buffers were sterile filtered prior to use.

Sensor Preparation: A new COOH2 sensor was installed and was conditioned by two 10 second injections of each of the following: 10 mM HCl, 50 mM NaOH, and 0.1% SDS. The instrument was primed in assay buffer. A solution containing 75 μg/mL Streptavidin, 4 mM EDC and 1 mM NHS in a 10 mM Acetate buffer pH 4.5 was injected over all three flow channels (FC) until ˜3000 RU of protein was captured. The sensor was deactivated by injecting 1 M Ethanolamine pH 8.0 for 4 min to cap unreacted NHS esters. A solution containing 500 nM SelSP1 in assay buffer was injected over FC3 and FC2 for 3 min. Approximately 300 RU were captured on FC2 and 250 RU were captured on FC3. FC1 was blocked by injecting 13 UM biotin-PEO4-NH2 for 2 min. The system was primed again in assay buffer.

CCL27 Binding Assay: The CCL27 binding assay consisted of four injections where the first inject was either buffer, 40 μg/mL K2 or 40 μg/mL SelK2; the second inject was 990 nM CCL27 in assay buffer; and the third and fourth injects were 0.1% SDS and 20 mM NaOH. Inject 1 was for 20 minute, inject 2 was 2 minutes and injects 3 and 4 were each 1 minute. Injects were flowed over all three FCs. Each assay cycle was repeated three times in sequential order.

Data Analysis: Data were processed and analysed using Qdat (BioLogic Software & SensiQ Technologies, Inc.). Assay curves were overlaid on the X axis and the Y axis was normalized at a time point directly before the CCL27 inject. The reference channel signal was subtracted from the SelSP1 channel signal to subtract non-specific artifacts. The identity of the blocking injection sample was used to differentiate the assay cycles and compare the CCL27 binding. The blocking injection samples were alternated, and each was repeated in triplicate. The replicates of each were compared together because an overall decrease in CCL27 binding was observed for each of the three blocking samples. So the Ab blocked CCL27 injects were referenced against the buffer blocked cycle for their respective replicate set. Percent inhibition was calculated using the CCL27 injection after buffer inject as a control for each Ab blocked replicate.

### Results

FIG. 6 shows the complete assay cycles for each blocking sample where the parental and SelK2 cycles (dot-dash line and dashed line curves, respectively) show large binding responses of antibody in injection ‘A’ while the solid line curves have negligible binding in this inject. FIG. 7 shows the same data zoomed into injection ‘B’ and re-normalized just before this injection. This view illustrates that the CCL27 binds SelSP1 with weak affinity (KD 10 μM, fit not shown) in the absence of antibody blocking (buffer control). This binding is decreased when the SelSP1 is blocked with either of the antibodies.

TABLE 5
Percent CCL27 Inhibition

| | Rep. 1 | Rep. 2 | Rep. 3 | Avg. | Std.Dev. |
|---|---|---|---|---|---|
| Blocking Ab | (%) | (%) | (%) | (%) | (±%) |
| Parental Ab | 57.1 | 50.6 | 53.5 | 50 | 5 |
| (FC2 data) | | | | | |
| Parental Ab | 50.5 | 43.1 | 47 | | |
| (FC3 data) | | | | | |
| SelK2 (FC2 | 67.2 | 61.9 | 68.3 | 62 | 6 |
| data) | | | | | |
| SelK2 (FC3 | 58.8 | 53.1 | 62.3 | | |
| data) | | | | | |

Table 5 shows the percent inhibition for the parental and SelK2 antibodies at each replicate and on two SelSP1 reaction FCs. SelK2 was found to inhibit CCL27 binding by 62+6%. The parental antibody was found to inhibit CCL27 binding by 50±5%. The slight increase in inhibition of SelK2 over K2 is most likely due to its ability to bind more to SelSP1 than K2. These results demonstrate the ability of the SelK2 antibody to block chemokine binding through PSGL-1 inhibition.

The inventors submit that this characteristic of SelK2's binding can be linked either to a conformation of binding which allows for better packing density of antibody or higher affinity toward the SelSP1 target.

Thus, this study demonstrates that these two antibodies which bind to SelSP1 can specifically block CCL27 binding to SelSP1, indicating that the CCL27 recognizes a site on SelSP1 which overlaps with its antibody binding sites. The affinity of both antibodies for SelSP1 is several orders of magnitude stronger than CCL27 and therefore, the likelihood of antibody displacing CCL27 in vivo is very high.

Taken together, the data of Examples 4 and 5 demonstrate PSGL-1 binding affinity, specificity, and functionality of the SelK2 antibody. These results also demonstrate a dual function for SelK2. The N-terminal domain of PSGL-1 contains a selectin binding domain that overlaps with a chemokine-binding domain. SelK2 binds to this domain and effectively blocks both selectin and chemokine binding.

### Example 6. SelK2 Effectively Inhibits Human Neutrophil Binding to P-selectin Under Flow

To determine the effectiveness of SelK2 in preventing the adhesive interactions between PSGL-1 and its main ligand P-selectin, human neutrophils were incubated with SelK2 prior to perfusing the cells over P-selectin coated plates under physiologic flow.

Human neutrophils were incubated with buffer alone (Control) or SelK2 at concentrations of 0.5, 0.25 or 0.125 g/mL for 5 minutes. The cells were then perfused over a surface coated with human P-selectin in a parallel-plate flow chamber under a shear stress of 1.0 dyn/cm2. Dynamic images of representative fields were recorded using a digital camera. Elongated streaks depict cells initially rolling on the surface of plate bound P-selectin while rounded cells represent cells that have transiently adhered to P-selectin.

As shown in FIG. 8, SelK2 inhibited neutrophil rolling on P-selectin in a dose dependent manner over a concentration range of 0.125 to 0.5 μg/mL, achieving near complete inhibition at 0.5 μg/mL.

### Example 7. Pharmacokinetic Analysis

### PK ELISA Assay

A PK ELISA assay was used to measure the concentration of SelK2 determined by a ligand binding antigen ELISA (LBA-ELISA) method. Following administration of 7.5 mg/kg Selk2 to subjects on Visit 4 Day 1 (Dose 1) and Visit 8 Day 22 (Dose 2), serum samples were collected for SelK2 analysis at time points: 15 minutes, 30 minutes, 1 hour, 24 hours, 8 days, 15 days, 29 days, 36 days, 43 days, 50 days, and 57 days post-administration, together with a pre-dose collection.

Neutravidin coated 96-well ELISA plates were loaded with biotinylated SP-1 peptide and diluted serum samples were then loaded and the SelK2 was captured. The SelK2 was then detected with a goat anti-human IgG that is HRP conjugated and a colorimetric signal can then be generated by developing with a TMB substrate. The concentration of SelK2 in serum samples is determined by measuring the sample signal to the signal of a known calibration curve containing multiple concentrations of SelK2.

As shown in FIG. 9 and Table 6, the mean and medium half-life is 322 and 268 hours, respectively. In addition, the mean and medium time of maximum observed serum concentration for dose 1 was 1.97 hours and 0.9 hours, respectively, and dose 2 was 4.84 hours and 1 hour, respectively.

TABLE 6
Descriptive Statistics of Serum Pharmacokinetic
Parameters of SelK2

| | SelK2 7.5 mg/kg | | | |
|---|---|---|---|---|
| Parameter (unit) | Dose 1 | | Dose 2 | |
| t_{1/2} (hours) | | | | |
| n | 16 | | 16 | |
| Mean (StdDev) | 322 | (240) | 322 | (195) |
| Median | 284 | | 252 | |
| Min, max | 111, 1167 | | 107, 855 | |
| tₘₐₓ (hours) | | | | |
| n | 18 | | 16 | |
| Mean (StdDev) | 1.974 | (5.4394) | 4.842 | (11.80) |
| Median | 0.90000 | | 1.000 | |
| Min, max | 0.2500, 24 | | 0.2667, 43 | |
| Cₘₐₓ (μg/mL) | | | | |
| n | 18 | | 16 | |
| Mean (StdDev) | 220 | (74.06) | 266 | (74.06) |
| Median | 223 | | 252 | |
| Min, max | 70, 345 | | 110, 413 | |
| AUC₀₋ₜ (h · μg/mL) | | | | |
| n | 18 | | 16 | |
| Mean (StdDev) | 35,785 | (12,467) | 63,508 | (19,193) |
| Median | 35,072 | | 65,501 | |
| Min, max | 10,174, 57,644 | | 27,001, 90,390 | |
| AUCₜₐᵤ (h · μg/mL) | | | | |
| n | — | | 16 | |
| Mean (StdDev) | — | | 50,941 | (14,074) |
| Median | — | | 51,008 | |
| Min, max | — | | 21,552, 69,043 | |
| AUC_{0-∞} (h · μg/mL) | | | | |
| n | 16 | | 16 | |
| Mean (StdDev) | 54,442 | (19,733) | 79,101 | (29,643) |
| Median | 54,072 | | 79,594 | |
| Min, max | 15,127, 95,721 | | 28,884, 122,968 | |
| CL (mL/h/kg) | | | | |
| n | 16 | | 16 | |
| Mean (StdDev) | 166 | (98.40) | 163 | (63.67) |
| Median | 139 | | 147 | |
| Min, max | 78, 496 | | 109, 348 | |
| Vz (mL/kg) | | | | |
| n | 16 | | 16 | |
| Mean (StdDev) | 66,879 | (42,310) | 72,273 | (47,655) |
| Median | 54,319 | | 56,367 | |
| Min, max | 36,736, 199,909 | | 28,132, 194,710 | |
| Rac | | | | |
| n | 16 | | 16 | |
| Mean (StdDev) | 0.84227 | (0.256026) | 0.91440 | (0.112111) |

| | | |
|---|---|---|
| Median | 0.94182 | 0.95553 |
| Min, max | 0.1658, 0.9997 | 0.5399, 0.9944 |

Abbreviations: AUC_{0-∞} = area under the serum concentration-time curve from time 0 to infinity; AUC₀₋ₜ = area under the serum concentration-time curve to the last quantified concentration; AUCₜₐᵤ = area under the serum concentration-time curve over the dosing interval; CL = serum clearance; Cₘₐₓ = maximum observed serum concentration; max = maximum; min = minimum; n = number of subjects with a measurement; Rac = accumulation of drug; t_{1/2} = terminal elimination half-life; StdDev = standard deviation; tₘₐₓ = time of maximum observed serum concentration; Vz = apparent volume of distribution.

### PD Assay

The PD assay was utilized to measure the relative inhibition of Psel-Ig binding to PSGL-1 (GSP6) by SelK2 in serum samples using Surface Plasmon Response technology.

A streptavidin coated gold sensor chip was first loaded with biotinylated GSP6 and diluted serum samples were injected allowing for active SelK2 to bind GSP6. Last, Psel-Ig is injected to bind any free GSP6 and the signal was assessed by the change in response units (RU) from before the Psel-Ig injection to 25 seconds following injection and non-specific signal was subtracted using a control, in-line reference channel consisting of streptavidin without GSP6. The relative inhibition is determined by comparing the Psel-Ig signal of a serum sample to the Psel-Ig signal of the negative control (the specific “pre-dose”).

As shown in FIG. 10, Psel-Ig is blocked concurrently in accordance with the increase observed in SelK2 serum concentration. When the mean serum concentration is 35-50 μg/mL, the percentage inhibition of Psel-Ig is above 80%; in this assay, 80% or above is considered to be completely blocked. Notably, a 58.6% inhibition of Psel-Ig is still achieved with the lowest serum concentration of 27.3 μg/ml at day 57 (+/−3 days) following dose 2.

### Example 8. Study to Assess the Safety and Efficacy of SelK2 on Airway Responses Following Allergen Challenge in Subjects with Asthma and COPD (ClinicalTrials.Gov Identifier: NCT04540042)

In a recent two part, randomised, double-blind, placebo-controlled, phase II parallel group study SelK2 was administered to humans. The main purpose of Part 1 of this study was to examine how safe and effective two doses of SelK2 was on participants with mild asthma. Lung function and inflammatory cell numbers were measured in response to the administration of an allergen into the lungs in the presence or absence of SelK2. Part 2 of this study examined how safe and effective one dose of SelK2 is on participants with chronic obstructive pulmonary disease (COPD). Lung function and inflammatory cell numbers were measured in COPD patients in the presence or absence of SelK2.

### Key Inclusion Criteria for Part 1:

Males or females, 18-65 years of age (inclusive);
Body Mass Index (BMI) ≥18.0 and ≤35.0 kg/m2.
Documented physician-diagnosed asthma for ≥4 months prior to screening.
Pre-bronchodilator FEV1≥70% predicted at screening.
Documented allergy to at least one common allergen as confirmed by the skin prick test.
Dual responder to inhaled bronchial allergen challenges as manifested by positive allergen-induced early (EAR) and late airway bronchoconstriction (LAR) at screening.

### Key Exclusion Criteria for Part 1:

Lung disease other than stable, mild asthma; e.g., worsening of asthma that requires a change in asthma therapy in the past 4 weeks or is deemed clinically significant by the investigator.
A diagnosed current or recent (within previous 8 weeks of screening, or prior to randomisation) bacterial, protozoal, viral or parasitic infection; is suspected of or is at high risk of having a parasitic infection, or has a history of more than one episode of herpes zoster infection.
Has a history of life-threatening asthma, defined as an asthma episode that required intubation and/or was associated with hypercapnea, respiratory arrest and/or hypoxic seizures.
Has been hospitalised or has attended the emergency room for asthma in the 12 months prior to screening, or prior to randomization.
A history of tuberculosis (latent or active) or systemic fungal diseases.

### Key Inclusion Criteria for Part 2:

Male or female, 40 to 75 years of age, inclusive, at the time of informed consent.
Confirmed diagnosis by a physician of COPD with symptoms compatible with COPD for at least 1 year prior to screening.
BMI≥18.0 and ≤35.0 kg/m2 at screening.
Able to tolerate sputum induction and produce an adequate sputum sample with a neutrophil differential count >55% at screening.
Post-bronchodilator FEV1≥30% and ≤80% of the predicted normal, and post-bronchodilator FEV1/FVC <0.7 at the time of Screening.
Current or former tobacco smoker who has a smoking history of at least 10 pack years (Ten pack-years are defined as 20 cigarettes a day for 10 years, or 10 cigarettes a day for 20 years).
Has a negative result in the blood test for tuberculosis (TB) at screening.

### Key Exclusion Criteria for Part 2:

COPD exacerbation requiring oral steroids and/or antibiotics, within the 8 weeks prior to screening or prior to randomisation.
A positive sputum culture at Screening indicating ongoing infection.
Other respiratory disorders: Subjects with a current diagnosis of asthma, active tuberculosis, lung cancer, bronchiectasis, sarcoidosis, lung fibrosis, interstitial lung diseases, known alpha-1 antitrypsin deficiency or other active pulmonary diseases other than COPD.
A history of life-threatening COPD including intensive care unit admission and/or requiring intubation within the last 5 years.
A history of >1 hospitalisation for COPD in the previous 1 year prior to screening.
Previous lung resection, lung reduction surgery or lung transplantation.
Requires supplemental oxygen, even on an occasional basis.
Any infection requiring hospitalisation or intravenous antibiotics within 6 months prior to Screening or prior to randomisation.
A diagnosed current or recent (within previous 8 weeks of screening, or prior to randomisation) bacterial, protozoal, viral or parasitic infection; is suspected of or is at high risk of having a parasitic infection, or has a history of more than one episode of herpes zoster infection.
Active participation in a pulmonary rehabilitation program.
A history of tuberculosis (latent or active) or systemic fungal diseases.

The bronchial allergen challenge was performed, as per standard procedures using the method described by Taylor and colleagues (Taylor, Harris, and O'Connor, 2000; Comparison of incremental and bolus dose inhaled allergen challenge in asthmatic patients; Clinical and Experimental Allergy, 30:56-63), to confirm the presence of an early and late phase response at the screening visit. The cumulative dose of allergen required to achieve a successful response at Screening phase was administered as a bolus during the treatment phase. Subjects that experienced a significant symptomatic fall in FEV1 during the EAR and/or LAR phases at screening (beyond the expected values of 20% and 15% respectively) were only randomised at the discretion of the Investigator. Alternatively, the subject may have repeated the bronchial allergen challenge at screening to achieve a lower cumulative dose of the same allergen or used a different allergen if deemed safe to do so by the Investigator. In the event that the subject met both the EAR and the LAR and a repeat allergen challenge was performed, the repeat challenge was at least 21 days later.

Time-points for FEV1 relative to the allergen challenge are listed below:

Screening: Pre-diluent, post-diluent, +5 minutes (multiple depending on number of allergen titrations), +10 minutes (multiple depending on number of allergen titrations), +15 minutes (multiple depending on number of allergen titrations), +20 minutes (multiple depending on number of allergen titrations), +30 minutes (multiple depending on number of allergen titrations), +45 minutes, 1, 1.5, 2, 2.5, 3, 3.5, 4, 4.5, 5, 5.5, 6, 6.5, 7, 7.5, 8 and 24 hours.

Treatment Phase: Pre-diluent, post-diluent, +5, 10, 15, 20, 30, 45 minutes, 1, 1.5, 2, 2.5, 3, 3.5, 4, 4.5, 5, 5.5, 6, 6.5, 7, 7.5, 8 and 24 hours.

Failure to achieve both an EAR and LAR during the allergen challenge conducted at Screening indicated that the subject is non-responsive to the allergen used in the challenge. As asthmatic subjects tend to be atopic to more than one type of air borne allergen, the allergen challenge may be repeated with a different allergen from that used in the initial challenge, if deemed safe by the Investigator. The repeated allergen challenge test, if needed, was performed at least 7 days later.

Spirometry (FEV1, FVC, FEV1% predicted, FVC % predicted, FEV1/FVC (reported as %), FEF25%-75%, FEF25%-75% % predicted) was measured using a spirometer that meets the American Thoracic Society (ATS)/European Respiratory Society (ERS) recommendations. Spirometry was performed in accordance with ATS/ERS 2019 criteria. Predicted normal values used are based on the Global Lung Function Initiative predicted values (GLI) (2012) (Quanjer, et al., 2012; Multi-ethnic reference values for spirometry for the 3-95-year age range: the global lung function 2012 equations; Eur Respir J; 40:1324-1343).

TABLE 7
Phase II Part 1 Study Design.

| | | | | | | | | | | | | | | End of |
|---|---|---|---|---|---|---|---|---|---|---|---|---|---|---|
| Screening | | | | | | | | | | | | | | Study |
| Phase | | | Treatment Phase | | | | | | | | | | | Visit |
| V1 | V2 | V3 | V4 | V5 | V6 | V7 | V8 | V9 | V10 | V11 | V12 | V13 | V14 | V15 |

| Day −42 to Day −1 | Day 1 | Day 2 | Day 8 | Day 15 | Day 22 | Day 23 | Day 29 | Day 36 | Day 37 | Day 43 | Day 50 | Day 57 |
|---|---|---|---|---|---|---|---|---|---|---|---|---|
| | | | (+/−1) | (+/−1) | (+/−2) | (V8 + 1) | (+/−2) | (+/−2) | (V11 + 1) | (+/−2) | (+/−2) | (+/−3 |
| Eligibility and | Study | | | | Study | | | Allergen | | | | |
| Allergen Dose | Drug | | | | Drug | | | Challenge | | | | |
| Determination | Dose | | | | Dose | | | Endpoints | | | | |

As outlined in Table 7, the Phase II part 1 study involved 42 days of screening prior to SelK2 or placebo treatment intravenously administered on days 1 and 22. This was followed by a subsequent allergen challenge on day 36. As outlined in Tables 8 and 9, the study was composed of 9 patients who received the SelK2 antibody treatment and 15 patients who received the placebo treatment. The primary and secondary endpoints were assessed on day 36.

As depicted in Tables 9 and 10, Subject Disposition and Demographics was fairly evenly distributed between SelK2- and Placebo-treated Patients.

TABLE 8
Subject Disposition (Part 1)

| | SelK2 | | |
|---|---|---|---|
| | 7.5 mg/kg | Placebo | Overall |
| All screened subjects | | | 114 |
| Enrolled set | | | 24 |
| Subjects who completed study | 8 (88.9) | 14 (93.3) | 22 (91.7) |
| treatment, n (%) | | | |
| Subjects who prematurely | 0 | 0 | 0 |
| discontinued study treatment, n (%) | | | |
| Subjects who completed the study, | 8 (88.9) | 15 (100) | 23 (95.8) |
| n (%) | | | |
| Subjects who prematurely | 1 (11.1) | 0 | 1 (4.2) |
| discontinued study, n (%) | | | |
| Reason for study withdrawal, n (%) | | | |
| Consent withdrawn | 1 (11.1) | 0 | 1 (4.2) |
| Study duration (days)^{a} | | | |
| N | 9 | 15 | 24 |
| Mean | 63.1 | 56.1 | 58.7 |
| StdDev | 20.95 | 0.70 | 12.85 |
| Minimum | 49 | 55 | 49 |
| Median | 56.0 | 56.0 | 56.0 |
| Maximum | 118 | 58 | 118 |

Abbreviations: n = the number of subjects meeting the criterion; StdDev = standard deviation. Study duration has been derived as the number of days between Study Day 1 and the date of study completion or the date of early study withdrawal.

TABLE 9
Demographic Characteristics (Part 1)

| Demographic | SelK2 7.5 mg/kg | | Placebo | | Overall | |
|---|---|---|---|---|---|---|
| Parameter | (N = 9) | | (N = 15) | | (N = 24) | |
| Sex, n (%) | | | | | | |
| Male | 8 | (88.9) | 13 | (86.7) | 21 | (87.5) |
| Female | 1 | (11.1) | 2 | (13.3) | 3 | (12.5) |
| Age (years)a | | | | | | |
| Mean (StdDev) | 34.9 | (9.88) | 34.2 | (10.68) | 34.5 | (10.17) |
| Median | 34 | | 33 | | 33 | |
| Min, Max | 23, 50 | | 21, 57 | | 21, 57 | |
| Race, n (%) | | | | | | |
| Black or African | 1 | (11.1) | 1 | (6.6) | 2 | (8.3) |
| American | | | | | | |
| Asian | 1 | (11.1) | 2 | (13.3) | 3 | (12.5) |
| White | 7 | (77.8) | 10 | (66.6) | 17 | (70.8) |
| Other | 0 | | 2 | (13.3) | 2 | (8.3) |
| Ethnic origin, n (%) | | | | | | |
| Not Hispanic | 9 | (100) | 15 | (100) | 24 | (100) |
| Hispanic | 0 | | 0 | | 0 | |
| BMI, kg/m² | | | | | | |
| Mean (StdDev) | 29.19 | (3.53) | 25.39 | (2.46) | 26.81 | (3.40) |
| Median | 29.56 | | 25.56 | | 26.72 | |
| Min, Max | 23.65, 33.96 | | 20.14, 28.86 | | 20.14, 33.96 | |
| Sputum producers at | | | | | | |
| screening, n (%) | | | | | | |
| Yes | 6 | (66.7) | 7 | (46.7) | 13 | (54.2) |
| No | 3 | (33.3) | 8 | (53.3) | 11 | (45.8) |

Abbreviations: BMI = body mass index; max = maximum; min = minimum; StdDev = standard deviation.
aAge is calculated in years from the date of first study treatment.
b. BMI is calculated as weight (kg)/height (m²).

FIG. 11 shows the allergen challenge response curve (EAR and LAR) of placebo patients during screening and during treatment. If the allergen challenge model is well controlled, the two curves should lay on top of one another as demonstrated in the figure. Table 10 and FIG. 12 show that treatment with SelK2 antibody in comparison to the placebo control reduced the area under the curve (AUC) of percentage fall in FEV1 between 3 and 8 hours post allergen challenge resulting in a mean percentage improvement in lung function of 45.7%. Moreover, as shown in Table 11 and FIG. 12, treatment with SelK2 antibody in comparison to the placebo control reduced the mean maximum percent fall between 3 and 8 hours post allergen challenge resulting in a mean improvement in lung function of 52.4%.

TABLE 10
Statistics of AUC of % fall in FEV1 between 3 and 8 hours

| Statistics | SelK2 | Placebo |
|---|---|---|
| N | 9 | 15 |
| LS Mean (SE) | 38.88 (18.026) | 71.58 (13.007) |
| Mean Difference (Placebo − SelK2) | 32.70 | |
| Standard Error | 22.621 | |
| 95% Confidence Interval for | −14.652, 80.043 | |
| Adjusted Difference | | |
| P-value for Zero Difference | 0.1647 | |
| Mean % Improvement | 45.7% | |

TABLE 11
Statistics of Maximum % fall in FEV1 between 3 and 8 hours

| Statistics | SelK2 | Placebo |
|---|---|---|
| N | 9 | 15 |
| LS Mean (SE) | 12.89 (5.210) | 27.10 (3.760) |
| Mean Difference (Placebo − SelK2) | 14.21 | |
| Standard Error | 6.538 | |
| 95% Confidence Interval for Adjusted | 0.523, 27.893 | |
| Difference | | |
| P-value for Zero Difference | 0.0426 | |
| Mean % Improvement | 52.4% | |

As shown in FIGS. 13 and 15, asthmatic patients who received the SelK2 treatment demonstrated a significant reduction in absolute differential eosinophil count and percentage differential eosinophil count respectively at 8 hours and 24 hours post-challenge in comparison to patients receiving the placebo treatment. FIG. 13 demonstrates that the mean absolute differential eosinophil count (10E6/g) was reduced from 1.15 to 0.44 at 8 hours post-challenge and from 1.36 to 0.47 at 24 hours post-challenge, in response to SelK2 treatment. FIG. 15 demonstrates that the percentage differential eosinophil count was reduced from 17.2% to 8.6% at 8 hours post-challenge and from 18.7% to 8.3% at 24 hours post-challenge in response to SelK2 treatment. In contrast, FIGS. 14 and 16 show that the reduction in eosinophils was not observed at 8 hours and 24 hours post-challenge in the blood of asthmatic patients. Thus, the reduction in eosinophils in the lungs with SelK2 treatment is not due to eosinophil depletion in the bloodstream that is characteristic of other approved therapies. Rather, the reduction of eosinophils in the lungs with SelK2 treatment is due to a decrease in extravasation of these cells across the endothelium and into the lungs.

This is supported by FIG. 17, which demonstrates that COPD patients who received the SelK2 treatment exhibited a significant reduction in absolute differential eosinophil count in sputum between days 8 and 28 of the study in comparison to patients receiving the placebo treatment. FIG. 17 demonstrates that at day 15, the mean absolute differential eosinophil count (10E6/g) was significantly reduced from 0.198 to 0.045; at day 22, the mean absolute differential eosinophil count (10E6/g) was significantly reduced from 0.229 to 0.016; and at day 28, the mean absolute differential eosinophil count (10E6/g) was reduced from 0.187 to 0.048.

Moreover, FIG. 18 shows that the reduction in eosinophils was not observed in the blood of COPD patients who received the SelK2 treatment.

Finally, FIG. 19 demonstrates how COPD patients who received the SelK2 treatment exhibited a significant reduction in absolute differential epithelial count in sputum following day 22 of the study in comparison to patients receiving the placebo treatment. At day 22, the mean absolute differential epithelial count (10E6/g) was significantly reduced from 0.350 to 0.038; at day 29, the mean absolute differential epithelial count (10E6/g) was significantly reduced from 0.286 to 0.128; and at day 43, the mean absolute differential epithelial count (10E6/g) was significantly reduced from 0.332 to 0.128.

Thus, this Phase II study confirms the role of SelK2 antibody in reducing eosinophil migration into the lungs resulting in an improvement in lung function post-allergen challenge in asthmatic patients. Importantly, the levels of eosinophils in the blood did not change, indicating that systemic immune suppression of these cells did not occur.

### Example 9. Comparative Analysis of Airway Response Following an Allergen Challenge in Patients with Stable Allergic Asthma Treated with SelK2 and Tezepelumab Antibodies

FIG. 20 is a comparative analysis demonstrating the percentage fall in FEV1 in patients with stable allergic asthma treated with the SelK2 antibody or the recently approved Tezepelumab antibody. Patients in both groups were subjected to an allergen challenge on day 36 in the case of SelK2 and on day 42 in the case of Tezepelumab. The Tezepelumab graph is taken from a previously published study that demonstrates the percentage FEV1 response in 16 patients with mild asthmatic patients treated with Tezepelumab antibody or a placebo control and subjected to an allergen challenge (Gauvreau, et al., 2014, Effects of an Anti-TSLP Antibody on Allergen-Induced Asthmatic Responses; The New England Journal of Medicine; 370:2102-2110).

As shown in Table 12 and FIG. 20, treatment with Tezepelumab antibody resulted in a 34% improvement in the maximum percentage fall in FEV1, a 33% improvement in FEV1 AUC, and a 4% improvement in average LAR percent fall. By contrast, SelK2 treatment resulted in a 55% improvement in the maximum percentage fall in FEV1, a 51% improvement in FEV1 AUC, and a 15% improvement in average LAR percent all. All three parameters of lung function following allergen challenge were superior with SelK2 treatment versus Tezepelumab treatment. In addition, both drugs resulted in a marked decrease of eosinophils in the lungs.

TABLE 12
SelK2 antibody and Tezepelumab antibody airway
response following an allergen challenge

| | SelK2 | Tezepelumab |
|---|---|---|
| Outcome | (Day 36) | (Day 42) |
| Maximum % Fall improvement | 55% | 34% |
| AUC Improvement | 51% | 33% |
| EOS Reduction | 60% | 75% |
| Average LAR % Fall Improvement | 14.9% | 4% |

### Example 10. Immunogenicity Analysis

Immune responses to the antibodies of the invention were investigated in recipients who had received SelK2 or placebo as part of the clinical trial.

Sera from each patient collected at the specified time points was tested in triplicate using an AlphaLISA format. Diluted sera was mixed with Donor and Acceptor beads previously coated with SelK2 Antibody. If anti-SelK2 antibodies are present in the sera, the anti-antibodies crosslink the two bead types, which causes an increase in fluorescence. Combined test results from pre-dose sera were used to establish the Positive/Negative cutoff. Triplicate measurements of pre-dose sera (collected prior to the initial loading dose) was used to calculate the mean and standard deviation of the measurement. The number of data points used to calculate the standard deviation was three times the number of pre-dose specimens collected. The 95% Positive/Negative cutoff was determined using the following formula: 95% Upper Cutoff=Average Signal+(1.96×Standard Deviation of all Signals)

### Results

The results of this analysis are set out in Table 13. As can be seen, incidences of immune responses were 27.8% of SelK2-treated subjects and 16.7% of placebo-treated subjects. Further, immune responses were of low levels, some of which did not repeat at later time points, and no neutralization of the activity of the antibody of the invention was observed.

TABLE 13
Immune responses to the SelK2 in comparison to placebo control.

| | SelK2 7.5 mg/kg (N = 18) | | Placebo (N = 18) | |
|---|---|---|---|---|
| | Subjects | Subjects | Subjects | Subjects |
| | Positive (%) | Analyzed (n) | Positive (%) | Analyzed (n) |
| Day 1 | 0 (0) | 18 | 0 | 18 |
| Day 15 | 1 (6.25) | 16 | 1 (5.56) | 18 |
| Day 22 | 0 (0) | 17 | 0 (0) | 18 |
| Day 29 | 0 (0) | 17 | 2 (11.11) | 18 |
| Day 43 | 1 (5.88) | 17 | 0 (0) | 18 |
| End of | 5 (29.41)* | 17 | 0 (0) | 18 |
| Study | | | | |

*Responses were of low levels, some of which did not repeat at later time points, and no neutralization of the activity of the antibody of the invention was observed.

### Example 11. Study to Evaluate the Effect of SelK2 on Absolute Cell Counts for Immune Cells in Sputum of COPD Patients

In a recent randomised, double-blind, placebo-controlled, parallel group study, the effect of SelK2 on sputum inflammatory cells in subjects with a diagnosis of COPD was investigated in comparison to placebo control. The main purpose of this study was to test the hypothesis that SelK2 will inhibit or disrupt extravasation of inflammatory cells into the airways of the lung in subjects with COPD and to evaluate the safety and efficacy of intravenously administered SelK2 compared with placebo. It was planned to recruit approximately 24 subjects with a diagnosis of COPD who had a sputum neutrophil count of >55% at Screening. Subjects were randomized in a 2:1 ratio to receive either 7.5 mg/kg SelK2 or placebo, respectively.

All subjects received a single dose of study drug on Day 1. The appropriate unit dose of SelK2 was prepared (by unblinded pharmacy staff or designee) as a SelK2 saline admixture to deliver the target dose to a subject administered a 100 mL infusion.

TABLE 14
Change from baseline in absolute numbers of eosinophils
and neutrophils in sputum and in total number of cells
per gram of sputum on Day 22 (21 days after dosing)

| | | | |
|---|---|---|---|
| | | | Treatment Difference |
| | SelK2 7.5 mg/kg | Placebo | (Placebo − SelK2 7.5 |
| Parameter | (N = 17) | (N = 8) | mg/kg) |
| Study Day | Mean (+/−SE) | Mean (+/−SE) | p-value¹ |

Absolute Eosinophil Count (10⁶/g)

| | | | |
|---|---|---|---|
| Baseline | 0.10 (+/−0.09) | 0.17 (+/−0.22) | — |
| Day 22 | 0.02 (+/−0.02) | 0.23 (+/−0.35) | 0.0181 |

Absolute Neutrophil Count (10⁶/g)

| | | | |
|---|---|---|---|
| Baseline | 4.1 (+/−3.1) | 5.8 (+/−8.4) | — |
| Day 22 | 1.7 (+/−1.4) | 5.8 (+/−7.5) | 0.0413 |

Total Number of Cells per Gram of Sputum (10⁶/g)

| | | | |
|---|---|---|---|
| Baseline | 4.8 (+/−0.8) | 7.1 (+/−3.5) | — |
| Day 22 | 2.0 (+/−1.5) | 7.4 (+/−9.7) | 0.0257 |

¹p-value was calculated based on the least squares mean.

As shown in FIGS. 21, 22 and 23 and in Table 14, statistically significant reductions from baseline were seen at Day 22 in absolute eosinophil and neutrophil cell counts and in total cells per gram of sputum in SelK2-treated subjects compared to placebo-treated subjects (p=0.0181, 0.0413 and 0.0257, respectively, based on adjusted least squares (LS) means).

Looking specifically at the reduction in absolute numbers of neutrophils in the SelK2 treatment group, cell counts went from a mean of 4.1+/−3.1×10⁶/g at baseline to 1.7+/−1.4×10⁶/g at Day 22, while cell counts in the placebo group were unchanged at a mean of 5.8+/−8.4×10⁶/g at baseline and 5.8+/−7.5×10⁶/g at Day 22 (p=0.0413, based on adjusted LS means).

Turning to the reduction in absolute numbers of eosinophils in the SelK2 treatment group, cell counts went from a mean of 0.10+/−0.09×10⁶/g at baseline to 0.02+/−0.02×10⁶/g at Day 22, while eosinophil counts in the placebo group increased from a mean of 0.17+/−0.22×10⁶/g at baseline to 0.23+/−0.35×10⁶/g at Day 22 (p=0.0181, based on adjusted LS means).

Similarly, total number of cells per gram of sputum in the SelK2 treatment group went from a mean of 4.8+/−0.8×10⁶/g at baseline to 2.0+/−1.5×10⁶/g at Day 22, while counts in the placebo group went from a mean of 7.1+/−3.5×10⁶/g at baseline to 7.4+/−9.7×10⁶/g at Day 22 (p=0.0257, based on adjusted LS means).

Neutrophils and eosinophils are both implicated in the pathogenesis of COPD, and so the ability to achieve targeted reduction of these cell types in the lungs with a single dose of SelK2 is encouraging. The chronic nature of COPD means that it is unexpectedly positive that a reduction of this sort is able to be achieved within the relatively short treatment period assessed. Prolonged treatment with SelK2 may be hypothesised to yield further beneficial effects.

While numbers of neutrophil and eosinophils present in the sputum of COPD patients were reduced, corresponding changes in numbers of neutrophils and eosinophils were not observed in the blood of SelK2 treatment group subjects. Without being bound by theory, this indicates that the reduction in neutrophils and eosinophils may result from the inhibition of extravasation of these cells into the lungs and not a reduction in the number of these cells in the circulation. Notably, previously published therapies have relied upon the depletion of these inflammatory cells in the bloodstream in order to achieve a desired depletion in the lungs. Since SelK2 specifically inhibits extravasation of these cells into the lungs, while leaving the cell counts in the blood intact it may be expected that SelK2 treatment will not adversely impact other beneficial functions of these cells in the blood.

### Sequence Information

TABLE 15
CDRs of SelK2

| | Kabat/ | | | |
|---|---|---|---|---|
| CDR | Chothia | Kabat | IMGT | Chothia |
| H1 | GFTFSSFG | SFGMH | GFTFSSFG | GFTFSSF |
| | MH | (SEQ ID | (SEQ ID | GMH |
| | (SEQ ID | NO: 9) | NO: 15) | (SEQ ID |
| | NO: 3) | | | NO: 44) |
| H2 | YISSASST | YISSASS | ISSASSTI | SSASST |
| | IYYADTVK | TIYYADT | (SEQ ID | (SEQ ID |
| | G | VKG | NO: 16) | NO: 45) |
| | (SEQ ID | (SEQ ID | | |
| | NO: 4) | NO: 10) | | |
| H3 | SYGY | SYGY | ASSYGY | SYGY |
| | (SEQ ID | (SEQ ID | (SEQ ID | (SEQ ID |
| | NO: 5) | NO: 11) | NO: 17) | NO: 46) |
| L1 | RSSRSLVH | RSSRSLV | RSLVHS | SRSLVHS |
| | SNGNTYLH | HSNGNTY | NGNTY | NGNTY |
| | (SEQ ID | LH | (SEQ ID | (SEQ ID |
| | NO: 6) | (SEQ ID | NO: 18) | NO: 47) |
| | | NO: 12) | | |
| L2 | KVSNRFS | KVSNRFS | KVS | KVS |
| | (SEQ ID | (SEQ ID | (SEQ ID | (SEQ ID |
| | NO: 7) | NO: 13) | NO: 19) | NO: 48) |
| L3 | SQTTHVP | SQTTHVP | SQTTHVP | TTHVPL |
| | LT | LT | LT | (SEQ ID |
| | (SEQ ID | (SEQ ID | (SEQ ID | NO: 49) |
| | NO: 8) | NO: 14) | NO: 20) | |

Sequence Information

| ID Number | Text Description | Biological Sequence |
|---|---|---|
| SEQ ID NO: 1 | SelK2 heavy | QVQLVESGGGVVQPGRSLRLSCAASGFTFSSFGMHWVRQ |
| | chain mature | APGKGLEWVAYISSASSTIYYADTVKGRFTISRDNSKNTLYL |
| | amino acid | QMNSLRAEDTAVYYCASSYGYWGQGTLVTVSSASTKGPS |
| | sequence | VFPLAPCSRSTSESTAALGCLVKDYFPEPVTVSWNSGALTS |
| | | GVHTFPAVLQSSGLYSLSSVVTVTSSNFGTQTYTCNVDHK |
| | | PSNTKVDKTVERKCCVECPPCPAPPVAGPSVFLFPPKPKD |
| | | TLMISRTPEVTCVVVDVSHEDPEVQFNWYVDGMEVHNAKT |
| | | KPREEQFNSTFRVVSVLTVVHQDWLNGKEYKCAVSNKGLP |
| | | APIEKTISKTKGQPREPQVYTLPPSREEMTKNQVSLTCLVK |
| | | GFYPSDIAVEWESNGQPENNYKTTPPMLDSDGSFFLYSKL |
| | | TVDKSRWQQGNVFSCSVMHEALHNHYTQKSLSLSPGK |
| SEQ ID NO: 2 | SelK2 light | DIVMTQTPLSLPVTPGEPASISCRSSRSLVHSNGNTYLHWY |
| | chain mature | LQKPGQSPQLLIYKVSNRFSGVPDRFSGSGSGTDFTLKISR |
| | amino acid | VEAEDVGVYYCSQTTHVPLTFGQGTRLEIKRTVAAPSVFIFP |
| | sequence | PSDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |
| SEQ ID NO: 3 | Kabat/Chothia- | GFTFSSFGMH |
| | defined heavy | |
| | chain CDR H1 | |
| SEQ ID NO: 4 | Kabat/Chothia- | YISSASSTIYYADTVKG |
| | defined heavy | |
| | chain CDR H2 | |
| SEQ ID NO: 5 | Kabat/Chothia- | SYGY |
| | defined heavy | |
| | chain CDR H3 | |
| SEQ ID NO: 6 | Kabat/Chothia- | RSSRSLVHSNGNTYLH |
| | defined light | |
| | chain CDR L1 | |
| SEQ ID NO: 7 | Kabat/Chothia- | KVSNRFS |
| | defined light | |
| | chain CDR L2 | |
| SEQ ID NO: 8 | Kabat/Chothia- | SQTTHVPLT |
| | defined light | |
| | chain CDR L3 | |
| SEQ ID NO: 9 | Kabat-defined | SFGMH |
| | heavy chain | |
| | CDR H1 | |
| SEQ ID NO: 10 | Kabat-defined | YISSASSTIYYADTVKG |
| | heavy chain | |
| | CDR H2 | |
| SEQ ID NO: 11 | Kabat-defined | SYGY |
| | heavy chain | |
| | CDR H3 | |
| SEQ ID NO: 12 | Kabat-defined | RSSRSLVHSNGNTYLH |
| | light chain CDR | |
| | L1 | |
| SEQ ID NO: 13 | Kabat-defined | KVSNRFS |
| | light chain CDR | |
| | L2 | |
| SEQ ID NO: 14 | Kabat-defined | SQTTHVPLT |
| | light chain CDR | |
| | L3 | |
| SEQ ID NO: 15 | IMGT-defined | GFTFSSFG |
| | heavy chain | |
| | CDR H1 | |
| SEQ ID NO: 16 | IMGT-defined | ISSASSTI |
| | heavy chain | |
| | CDR H2 | |
| SEQ ID NO: 17 | IMGT-defined | ASSYGY |
| | heavy chain | |
| | CDR H3 | |
| SEQ ID NO: 18 | IMGT-defined | RSLVHSNGNTY |
| | light chain CDR | |
| | L1 | |
| SEQ ID NO: 19 | IMGT-defined | KVS |
| | light chain CDR | |
| | L2 | |
| SEQ ID NO: 20 | IMGT-defined | SQTTHVPLT |
| | light chain CDR | |
| | L3 | |
| SEQ ID NO: 21 | Parental | DVQLVESGGGLVQPGGSRKLSCAASGFTFSSFGMHWVRQ |
| | antibody heavy | APEKGLEWVAYISSASSTIYYADTVKGRFTISRDNPKNTLFL |
| | chain variable | QMTSLRSEDTAMYYCASSYGYWGQGTTLTVSS |
| | region amino | |
| | acid sequence | |
| SEQ ID NO: 22 | Parental | DVVMTQTPLSLPVSLGDQASISCRSSRSLVHSNGNTYLHW |
| | antibody light | YLQKPGQSPQLLIYKVSNRFSGVPDRFSGSGSGTDFTLKIS |
| | chain variable | RVEAEDLGVYFCSQTTHVPLTFGAGTRLELKR |
| | region amino | |
| | acid sequence | |
| SEQ ID NO: 23 | VH | QVQLVESGGGVVQPGRSLRLSCAASGFTFSSYGMHWVRQ |
| | humanization | APGKGLEWVAVISYDGSNKYYADSVKGRFTISRDNSKNTLY |
| | acceptor | LQMNSLRAEDTAVYYCAK--YFDYWGQGTLVTVSS |
| | sequence: | |
| | germline VH3 1- | |
| | 3 3-30.5 | |
| SEQ ID NO: 24 | VL | DIVMTQTPLSLPVTPGEPASISCRSSQSLLDSDDGNTYLDW |
| | humanization | YLQKPGQSPQLLIYTLSYRASGVPDRFSGSGSGTDFTLKIS |
| | acceptor | RVEAEDVGVYYCMQRIEFP-ITFGQGTRLEIKR |
| | sequence: | |
| | germline | |
| | IGKV2-40*01 | |
| SEQ ID NO: 25 | SelK2 VH-Graft | QVQLVESGGGVVQPGRSLRLSCAASGFTFSSFGMHWVRQ |
| | | APGKGLEWVAYISSASSTIYYADTVKGRFTISRDNSKNTLYL |
| | | QMNSLRAEDTAVYYCAKSYGYWGQGTLVTVSS |
| SEQ ID NO: 26 | SelK2 VH-B1 | QVQLVESGGGVVQPGRSLRLSCAASGFTFSSFGMHWVRQ |
| | | APGKGLEWVAYISSASSTIYYADTVKGRFTISRDNSKNTLYL |
| | | QMNSLRAEDTAVYYCASSYGYWGQGTLVTVSS |
| SEQ ID NO: 27 | SelK2 VH-B2 | QVQLVESGGGVVQPGRSLRLSCAASGFTFSSFGMHWVRQ |
| | | APGKGLEWVAYISSASSTIYYADTVKGRFTISRDNPKNTLYL |
| | | QMNSLRAEDTAVYYCASSYGYWGQGTLVTVSS |
| SEQ ID NO: 28 | SelK2 VH-B5 | QVQLVESGGGVVQPGGSRRLSCAASGFTFSSFGMHWVR |
| | | QAPEKGLEWVAYISSASSTIYYADTVKGRFTISRDNPKNTLY |
| | | LQMNSLRAEDTAVYYCASSYGYWGQGTLVTVSS |
| SEQ ID NO: 29 | SelK2 VL-Graft | DIVMTQTPLSLPVTPGEPASISCRSSRSLVHSNGNTYLHWY |
| | | LQKPGQSPQLLIYKVSNRFSGVPDRFSGSGSGTDFTLKISR |
| | | VEAEDVGVYYCSQTTHVPLTFGQGTRLEIKR |
| SEQ ID NO: 30 | SelK2 VL-B2 | DIVMTQTPLSLPVTLGEQASISCRSSRSLVHSNGNTYLHWY |
| | | LQKPGQSPQLLIYKVSNRFSGVPDRFSGSGSGTDFTLKISR |
| | | VEAEDVGVYYCSQTTHVPLTFGQGTRLEIKR |
| SEQ ID NO: 31 | SelK2 VL-B4 | DIVMTQTPLSLPVTLGEQASISCRSSRSLVHSNGNTYLHWY |
| | | LQKPGQSPQLLIYKVSNRFSGVPDRFSGSGSGTDFTLKISR |
| | | VEAEDVGVYFCSQTTHVPLTFGAGTRLEIKR |
| SEQ ID NO: 32 | FR H1 | QVQLVESGGGVVQPGRSLRLSCAAS |
| SEQ ID NO: 33 | FR H2 | WVRQAPGKGLEWVA |
| SEQ ID NO: 34 | FR H3 | RFTISRDNSKNTLYLQMNSLRAEDTAVYYCAS |
| SEQ ID NO: 35 | FR H4 | WGQGTLVTVSS |
| SEQ ID NO: 36 | FR L1 | DIVMTQTPLSLPVTPGEPASISC |
| SEQ ID NO: 37 | FR L2 | WYLQKPGQSPQLLIY |
| SEQ ID NO: 38 | FR L3 | GVPDRFSGSGSGTDFTLKISRVEAEDVGVYYC |
| SEQ ID NO: 39 | FR L4 | FGQGTRLEIKR |
| SEQ ID NO: 40 | Amino acid | ASTKGPSVFPLAPCSRSTSESTAALGCLVKDYFPEPVTVSW |
| | sequence of | NSGALTSGVHTFPAVLQSSGLYSLSSVVTVTSSNFGTQTYT |
| | human IgG2 | CNVDHKPSNTKVDKTVERKCCVECPPCPAPPVAGPSVFLF |
| | heavy chain | PPKPKDTLMISRTPEVTCVVVDVSHEDPEVQFNWYVDGME |
| | constant region | VHNAKTKPREEQFNSTFRVVSVLTVVHQDWLNGKEYKCAV |
| | incorporating | SNKGLPAPIEKTISKTKGQPREPQVYTLPPSREEMTKNQVS |
| | mutation) | LTCLVKGFYPSDIAVEWESNGQPENNYKTTPPMLDSDGSF |
| | | FLYSKLTVDKSRWQQGNVFSCSVMHEALHNHYTQKSLSLS |
| | | PGK |
| SEQ ID NO: 41 | Amino acid | TVAAPSVFIFPPSDEQLKSGTASVVCLLNNFYPREAKVQWK |
| | sequence of | VDNALQSGNSQESVTEQDSKDSTYSLSSTLTLSKADYEKH |
| | human kappa | KVYACEVTHQGLSSPVTKSFNRGEC |
| | light chain | |
| | constant region | |
| SEQ ID NO: 42 | SelK2 heavy | atg ggc tgg tcc tgc atc atc ctg ttt ctg gtc gct acc gcc acc ggc |
| | chain DNA | gtg cac tcc caa gta caa ctg gtc gaa tct ggc ggc gga gtc gta |
| | sequence | caa cct ggc cgg tcc ctg aga ctg tct tgc gcc gcc tcc ggc ttc acc |
| | | ttc tcg agc ttc ggc atg cac tgg gtc cga caa gcc cct ggc aag |
| | | ggc ctg gag tgg gtc gcc tac atc tcc tcc gcc tcg agc acc atc tac |
| | | tac gcc gac acc gtg aag ggg aga ttc acc atc tcc cgg gac aac |
| | | tcc aaa aac acc ctg tac ctg caa atg aac tcc ctg cgg gcc gag |
| | | gac acc gcc gtg tac tac tgc gcc tcc tcc tac ggc tac tgg ggc caa |
| | | ggc acc ctc gtt act gtc tcc tct gcc tcc acc aag ggc ccc tcc gtg |
| | | ttc cct ctg gcc cct tgc tcc cgg tcc acc tcc gag tct acc gcc gct |
| | | ctg ggc tgc ctg gtc aag gac tac ttc ccc gag ccc gtg acc gtg tcc |
| | | tgg aac tct ggc gcc ctt acg agc ggc gtg cac acc ttc cct gcc gtg |
| | | ctg caa tcc tcc ggc ctg tac tcc ctg tcc tcc gtc gtc acc gtg acc |
| | | tcg agc aac ttc ggc acc caa acc tac acc tgt aac gtg gac cac |
| | | aaa ccc tcc aac acc aag gtc gac aaa acc gtg gaa cgg aag tgc |
| | | tgc gtg gag tgc ccc ccc tgc cct gct cct cct gtg gct ggg ccg agc |
| | | gtg ttc ctg ttc ccc cca aaa ccc aag gac acc ctg atg atc tcc cgg |
| | | acc ccc gag gtc acc tgc gtc gtc gtg gac gtg tcc cac gag gac |
| | | ccc gag gtc caa ttc aat tgg tac gtg gac ggc atg gag gtc cac |
| | | aac gcc aaa acc aaa ccg aga gag gaa caa ttc aac tcc acc ttc |
| | | cgc gtc gtg tct gtg ctt acc gtc gtg cac caa gac tgg ctg aac ggc |
| | | aaa gaa tac aag tgc gct gtc tcc aac aag ggc ctg cct gcc ccc |
| | | atc gaa aaa acc atc tcc aaa acc aag ggc caa ccc cgc gag ccc |
| | | caa gtc tac aca ctg ccc ccg agc cgg gag gag atg acc aaa aac |
| | | caa gtc tcc ctt acc tgt ctc gtg aaa ggc ttc tac ccc tcc gat atc |
| | | gcc gtg gag tgg gag tcc aac ggc caa ccc gag aac aac tac aaa |
| | | acc acc ccc ccc atg ctg gac tcc gac ggc tca ttc ttc ctg tac tcc |
| | | aaa ctt acc gtc gac aaa tcc cgc tgg caa caa ggc aac gtg ttc |
| | | tcc tgc tcc gtg atg cac gag gcc ctg cac aac cac tac acc caa |
| | | aaa tcc ctg tcc ctg agc ccc ggc aag tga |
| SEQ ID NO: 43 | SelK2 light | atg ggc tgg tcc tgc atc atc ctg ttt ctg gtg gct acc gcc acc ggc |
| | chain DNA | gtg cac tcc gac atc gtg atg acc cag acc ccc ctg tcc ctg ccc gtg |
| | sequence | aca cct ggc gag cct gcc tcc atc tcc tgc cgg tcc tcc cgg tcc ctg |
| | | gtg cac tcc aac ggc aac acc tac ctg cac tgg tat ctg cag aag |
| | | ccc ggc cag tcc ccc cag ctg ctg atc tac aag gtg tcc aac cgg ttc |
| | | tcc ggc gtg ccc gac aga ttc tcc ggc tcc ggc tct ggc acc gac ttc |
| | | acc ctg aag atc tcc cgg gtg gaa gcc gag gac gtg ggc gtg tac |
| | | tac tgc tcc cag acc acc cac gtg ccc ctg acc ttc ggc cag ggc |
| | | acc cgg ctg gaa atc aag cgg acc gtg gcc gct ccc tcc gtg ttc atc |
| | | ttc cca ccc tcc gac gag cag ctg aag tcc ggc acc gcc tcc gtg |
| | | gtc tgc ctg ctg aac aac ttc tac ccc cgc gag gcc aag gtg cag |
| | | tgg aag gtg gac aac gcc ctg cag tcc ggc aac tcc caa gaa tcc |
| | | gtg acc gag cag gac tcc aag gac agc acc tac tcc ctg tcc tcc |
| | | acc ctg acc ctg tcc aag gcc gac tac gag aag cac aag gtg tac |
| | | gcc tgc gaa gtg acc cac cag ggc ctg tcc agc ccc gtg acc aag |
| | | tcc ttc aac cgg ggc gag tgc tga |
| SEQ ID NO: 44 | Chothia-defined | GFTFSSFGMH |
| | heavy chain | |
| | CDR H1 | |
| SEQ ID NO: 45 | Chothia-defined | SSASST |
| | heavy chain | |
| | CDR H2 | |
| SEQ ID NO: 46 | Chothia-defined | SYGY |
| | heavy chain | |
| | CDR H3 | |
| SEQ ID NO: 47 | Chothia-defined | SRSLVHSNGNTY |
| | light chain CDR | |
| | L1 | |
| SEQ ID NO: 48 | Chothia-defined | KVS |
| | light chain CDR | |
| | L2 | |
| SEQ ID NO: 49 | Chothia-defined | TTHVPL |
| | light chain CDR | |
| | L3 | |

### ASPECTS AND EMBODIMENTS OF THE INVENTION

The following paragraphs do not constitute claims, but do define aspects and embodiments of the present invention in respect of which protection may be sought.
1. A humanised anti-PSGL-1 antibody comprising at least one CDR of the antibody heavy chain set out in SEQ ID NO: 1 and at least one CDR of the antibody light chain set out in SEQ ID NO: 2.
2. The humanised anti-PSGL-1 antibody of paragraph 1, wherein the CDR H1 of the antibody comprises or consist of a sequence selected from the group consisting of: SEQ ID NO: 3; SEQ ID NO: 9; SEQ ID NO: 15; and SEQ ID NO: 44.
3. The humanised anti-PSGL-1 antibody of paragraph 1 or paragraph 2, wherein the CDR H2 of the antibody comprises or consist of a sequence selected from the group consisting of: SEQ ID NO: 4; SEQ ID NO: 10; SEQ ID NO: 16; and SEQ ID NO: 45.
4. The humanised anti-PSGL-1 antibody of any of paragraphs 1 to 3, wherein the CDR H3 of the antibody comprises or consist of a sequence selected from the group consisting of: SEQ ID NO: 5; SEQ ID NO: 11; SEQ ID NO: 17; and SEQ ID NO: 46.
5. The humanised anti-PSGL-1 antibody of any of paragraphs 1 to 4, wherein the CDR L1 of the antibody comprises or consist of a sequence selected from the group consisting of: SEQ ID NO: 6; SEQ ID NO: 12; SEQ ID NO: 18; and SEQ ID NO: 47.
6. The humanised anti-PSGL-1 antibody of any of paragraphs 1 to 5, wherein the CDR L2 of the antibody comprises or consist of a sequence selected from the group consisting of: SEQ ID NO: 7; SEQ ID NO: 13; SEQ ID NO: 19; and SEQ ID NO: 48.
7. The humanised anti-PSGL-1 antibody of any of paragraphs 1 to 6, wherein the CDR L3 of the antibody comprises or consist of a sequence selected from the group consisting of: SEQ ID NO: 8; SEQ ID NO: 14; SEQ ID NO: 20; and SEQ ID NO: 49.
8. The humanised anti-PSGL-1 antibody of any of paragraphs 1 to 7, comprising each of a Kabat/Chothia-defined CDR H1 of SEQ ID NO: 3, a Kabat/Chothia-defined CDR H2 of SEQ ID NO: 4, a Kabat/Chothia-defined CDR H3 of SEQ ID NO: 5, a Kabat/Chothia-defined CDR L1 of SEQ ID NO: 6, a Kabat/Chothia-defined CDR L2 of SEQ ID NO: 7, and a Kabat/Chothia-defined CDR L3 of SEQ ID NO: 8.
9. The humanised anti-PSGL-1 antibody of any of paragraphs 1 to 7, comprising each of a Kabat-defined CDR H1 of SEQ ID NO: 9, a Kabat-defined CDR H2 of SEQ ID NO: 10, a Kabat-defined CDR H3 of SEQ ID NO: 11, a Kabat-defined CDR L1 of SEQ ID NO: 12, a Kabat-defined CDR L2 of SEQ ID NO: 13, and a Kabat-defined CDR L3 of SEQ ID NO: 14.
10. The humanised anti-PSGL-1 antibody of any of paragraphs 1 to 7, comprising each of an IMGT-defined CDR H1 of SEQ ID NO: 15, an IMGT-defined CDR H2 of SEQ ID NO: 16, an IMGT-defined CDR H3 of SEQ ID NO: 17, an IMGT-defined CDR L1 of SEQ ID NO: 18, an IMGT-defined CDR L2 of SEQ ID NO: 19, and an IMGT-defined CDR L3 of SEQ ID NO: 20.
11. The humanised anti-PSGL-1 antibody of any of paragraphs 1 to 7, comprising each of a Chothia-defined CDR H1 of SEQ ID NO: 44, a Chothia-defined CDR H2 of SEQ ID NO: 45, a Chothia-defined CDR H3 of SEQ ID NO: 46, a Chothia-defined CDR L1 of SEQ ID NO: 47, a Chothia-defined CDR L2 of SEQ ID NO: 48, and a Chothia-defined CDR L3 of SEQ ID NO: 49.
12. The humanised anti-PSGL-1 antibody of any of paragraphs 1 to 11, comprising a heavy chain variable region with an amino acid sequence that comprises no more than 10 amino acid alterations as compared to the sequence set out in SEQ ID NO: 26.
13. The humanised anti-PSGL-1 antibody of any of paragraphs 1 to 12, comprising a heavy chain variable region that shares at least 95% identity with the heavy chain variable region amino acid sequence set out in SEQ ID NO: 26.
14. The humanised anti-PSGL-1 antibody of any of paragraphs 1 to 13, comprising a heavy chain comprising a variable sequence as set out in SEQ ID NO: 26.
15. The humanised anti-PSGL-1 antibody of any of paragraphs 1 to 14, comprising a light chain variable region with an amino acid sequence that comprises no more than 10 amino acid alterations as compared to the sequence set out in SEQ ID NO: 29.
16. The humanised anti-PSGL-1 antibody of any of paragraphs 1 to 15, comprising a light chain variable region that shares at least 95% identity with the light chain variable region amino acid sequence set out in SEQ ID NO: 29.
17. The humanised anti-PSGL-1 antibody of any of paragraphs 1 to 16, comprising a light chain comprising a variable sequence as set out in SEQ ID NO: 29.
18. An anti-PSGL-1 antibody:
comprising the heavy chain variable sequence set out in SEQ ID NO: 26 and the light chain variable sequence set out in SEQ ID NO: 29; or
comprising the heavy chain sequence set out in SEQ ID NO: 1 and the light chain sequence set out in SEQ ID NO: 2.
19. The anti-PSGL-1 antibody of any of paragraphs 1 to 18, wherein the antibody has an affinity for PSGL-1 of below 10 nM.
20. The anti-PSGL-1 antibody of paragraph 19, wherein the antibody has an affinity for PSGL-1 of below 5 nM.
21. The anti-PSGL-1 antibody of paragraph 20, wherein the antibody has an affinity for PSGL-1 of approximately 4.29 nM.
22. The anti-PSGL-1 antibody of any of paragraphs 1 to 21, wherein the antibody is able to inhibit and disrupt binding of PSGL-1 to its ligands.
23. The anti-PSGL-1 antibody of any of paragraphs 1 to 22, wherein the antibody is able to improve lung function in asthmatic patients by at least 35% as compared to placebo.
24. The anti-PSGL-1 antibody of paragraph 23, wherein the antibody is able to improve lung function in asthmatic patients by approximately 50% as compared to placebo.
25. A pharmaceutical composition comprising a humanised anti-PSGL-1 antibody comprising at least one CDR of the antibody heavy chain set out in SEQ ID NO: 1 and at least one CDR of the antibody light chain set out in SEQ ID NO: 2 and a pharmaceutically acceptable excipient.
26. The pharmaceutical composition of paragraph 25, wherein the antibody is as defined in any of paragraphs 1 to 24.
27. A method of preventing or treating a disease or condition in a subject in need thereof, the method comprising providing a therapeutically effective amount of an anti-PSGL-1 antibody to the subject, wherein the antibody comprises at least one CDR of the antibody heavy chain set out in SEQ ID NO: 1 and at least one CDR of the antibody light chain set out in SEQ ID NO: 2.
28. The method of preventing or treating a disease or condition of paragraph 27, wherein the antibody is as defined in any of paragraphs 1 to 24.
29. The method of preventing or treating a disease or condition of paragraph 27 or paragraph 2, wherein the antibody is provided in a pharmaceutical composition of paragraph 25 or paragraph 26.
30. The method of preventing or treating a disease or condition of any of paragraphs 27 to 29, for the prevention or treatment of an inflammatory condition or disease selected from the group consisting of: asthma; chronic obstructive pulmonary disease; allergic reactions; inflammatory bowel disease (e.g., Crohn's disease, ulcerative colitis, enteritis); arthritis (e.g., rheumatoid arthritis, osteoarthritis, psoriatic arthritis); graft rejection; graft versus host disease; psoriasis; dermatitis; nephritis; lupus erythematosus; scleroderma; rhinitis; anaphylaxis; diabetes; multiple sclerosis; atherosclerosis; and thyroiditis.
31. The method of treatment of any of paragraphs 27 to 30, for the prevention or treatment of a respiratory disease or condition selected from the group consisting of asthma and COPD.
32. The method of treatment of paragraph 31, for the prevention or treatment of asthma.
33. The method of treatment of paragraph 31, for the prevention or treatment of COPD.
34. The method of treatment of any of paragraphs 27 to 29, for the prevention or treatment of an eosinophilic condition or disease selected from the group consisting of: eosinophilic asthma; allergies; eosinophilic oesophagitis; eosinophilic dermatitis; acute myelogenous leukemia (AML), ascariasis; atopic dermatitis (eczema); bullous pemphigoid; cancer (such as Hodgkin lymphoma, leukemia, and certain myeloproliferative neoplasms); Churg-Strauss syndrome; drug allergy; eosinophilic cardiomyopathy; eosinophilic cellulitis (Wells' syndrome); eosinophilic colitis; eosinophilic enteritis; eosinophilic fasciitis; eosinophilic gastrointestinal diseases; eosinophilic granulomatosis with polyangiitis (EGPA); eosinophilic leukemia; eosinophilic myocarditis; hay fever (allergic rhinitis); Hodgkin's lymphoma (Hodgkin's disease); hypereosinophilic syndromes; idiopathic hypereosinophilic syndrome (HES); IgG4-Related Disease; inflammatory bowel disease (Crohn's disease, ulcerative colitis); lymphatic filariasis; neuromyelitis optica (NMO); ovarian cancer; parasitic infection; primary biliary cirrhosis; primary immunodeficiency; or trichinosis.
35. The method of treatment of paragraph 34, for the prevention or treatment of an eosinophilic condition or disease selected from the group consisting of: eosinophilic asthma; eosinophilic oesophagitis; and eosinophilic dermatitis.
36. An anti-PSGL-1 antibody comprising at least one CDR of the antibody heavy chain set out in SEQ ID NO: 1 and at least one CDR of the antibody light chain set out in SEQ ID NO: 2, for use as a medicament.
37. An anti-PSGL-1 antibody for use according to paragraph 36, wherein the antibody is as defined in any of paragraphs 1 to 24.
38. An anti-PSGL-1 antibody for use according to paragraph 36 or paragraph 37, wherein the antibody is provided in a pharmaceutical composition of paragraph 25 or paragraph 26.
39. An anti-PSGL-1 antibody for use according to any of paragraphs 36 to 38, wherein the antibody is for use in the prevention or treatment of an inflammatory condition or disease selected from the group consisting of: asthma; chronic obstructive pulmonary disease; allergic reactions; inflammatory bowel disease (e.g., Crohn's disease, ulcerative colitis, enteritis); arthritis (e.g., rheumatoid arthritis, osteoarthritis, psoriatic arthritis); graft rejection; graft versus host disease; psoriasis; dermatitis; nephritis; lupus erythematosus; scleroderma; rhinitis; anaphylaxis; diabetes; multiple sclerosis; atherosclerosis; and thyroiditis.
40. An anti-PSGL-1 antibody for use according to any of paragraphs 36 to 39, wherein the antibody is for use in the prevention or treatment of a respiratory disease or condition selected from the group consisting of: asthma and COPD.
41. An anti-PSGL-1 antibody for use according to paragraph 40, wherein the antibody is for use in the prevention or treatment of asthma.
42. An anti-PSGL-1 antibody for use according to paragraph 40, wherein the antibody is for use in the prevention or treatment of COPD.
43. An anti-PSGL-1 antibody for use according to any of paragraphs 36 to 38, wherein the antibody is for use in the prevention or treatment of an eosinophilic condition or disease selected from the group consisting of: eosinophilic asthma; allergies; eosinophilic oesophagitis; eosinophilic dermatitis; acute myelogenous leukemia (AML), ascariasis; atopic dermatitis (eczema); bullous pemphigoid; cancer (such as Hodgkin lymphoma, leukemia, and certain myeloproliferative neoplasms); Churg-Strauss syndrome; drug allergy; eosinophilic cardiomyopathy; eosinophilic cellulitis (Wells' syndrome); eosinophilic colitis; eosinophilic enteritis; eosinophilic fasciitis; eosinophilic gastrointestinal diseases; eosinophilic granulomatosis with polyangiitis (EGPA); eosinophilic leukemia; eosinophilic myocarditis; hay fever (allergic rhinitis); Hodgkin's lymphoma (Hodgkin's disease); hypereosinophilic syndromes; idiopathic hypereosinophilic syndrome (HES); IgG4-Related Disease; inflammatory bowel disease (Crohn's disease, ulcerative colitis); lymphatic filariasis; neuromyelitis optica (NMO); ovarian cancer; parasitic infection; primary biliary cirrhosis; primary immunodeficiency; or trichinosis.
44. An anti-PSGL-1 antibody for use according to paragraph 43, wherein the antibody is for use in the prevention or treatment of an eosinophilic condition or disease selected from the group consisting of: eosinophilic asthma; eosinophilic oesophagitis; and eosinophilic dermatitis.
45. A polynucleotide sequence encoding an immunoglobulin chain of a humanised anti-PSGL-1 antibody comprising at least one CDR of the antibody heavy chain set out in SEQ ID NO: 1 and at least one CDR of the antibody light chain set out in SEQ ID NO: 2.
46. The polynucleotide sequence of paragraph 45, wherein the sequence encodes an immunoglobulin chain of a humanised anti-PSGL-1 antibody according to any of paragraphs 1 to 23.
47. The polynucleotide sequence of paragraph 45 or paragraph 46 comprising SEQ ID NO: 42.
48. The polynucleotide sequence of any of paragraphs 45 to 47 comprising SEQ ID NO: 43.
49. A cell comprising a polynucleotide sequence encoding an immunoglobulin chain of a humanised anti-PSGL-1 antibody comprising at least one CDR of the antibody heavy chain set out in SEQ ID NO: 1 and at least one CDR of the antibody light chain set out in SEQ ID NO: 2.
50. The cell of paragraph 49, wherein the polynucleotide sequence is as defined in any of paragraphs 46 to 48.
51. A method of manufacturing a humanised anti-PSGL-1 antibody comprising at least one CDR of the antibody heavy chain set out in SEQ ID NO: 1 and at least one CDR of the antibody light chain set out in SEQ ID NO: 2, the method comprising:
expressing a first polynucleotide sequence encoding an immunoglobulin chain of a humanised anti-PSGL-1 antibody comprising at least one CDR of the antibody heavy chain set out in SEQ ID NO: 1; and
expressing a second polynucleotide sequence encoding an immunoglobulin chain of a humanised anti-PSGL-1 antibody comprising at least one CDR of the antibody light chain set out in SEQ ID NO: 2.
52. The method of paragraph 51, wherein the first and/or second polynucleotide sequence is as defined in any of paragraphs 46 to 48.

1-18. (canceled)

19. An anti-PSGL-1 antibody
comprising a heavy chain variable domain sequence set out in SEQ ID NO: 26 and a light chain variable domain sequence set out in SEQ ID NO: 29.

20. The anti-PSGL-1 antibody of claim 19, wherein the antibody has an affinity for PSGL-1 of below 10 nM.

21. The anti-PSGL-1 antibody of claim 20, wherein the antibody has an affinity for PSGL-1 of below 5 nM.

22. The anti-PSGL-1 antibody of claim 21, wherein the antibody has an affinity for PSGL-1 of approximately 4.29 nM.

23. The anti-PSGL-1 antibody of claim 19, wherein the antibody is able to inhibit and disrupt binding of PSGL-1 to its ligands.

24. The anti-PSGL-1 antibody of claim 19, wherein the antibody is able to improve lung function in asthmatic patients by at least 35% as compared to placebo.

25. The anti-PSGL-1 antibody of claim 24, wherein the antibody is able to improve lung function in asthmatic patients by approximately 50% as compared to placebo.

26-29. (canceled)

30. A method of preventing or treating a disease or condition in a subject in need thereof, the method comprising providing a therapeutically effective amount of the anti-PSGL-1 antibody of claim 19 to the subject.

31-33. (canceled)

34. The method of claim 30, wherein the disease is an inflammatory condition or disease.

35-37. (canceled)

38. The method of claim 30, wherein the disease or condition is an eosinophilic condition or disease.

39. (canceled)

40. The method of claim 30, wherein the disease or condition is a neutrophilic condition or disease.

41-84. (canceled)

85. The anti-PSGL-1 antibody of claim 19 comprising a heavy chain sequence set out in SEQ ID NO: 1 and a light chain sequence set out in SEQ ID NO: 2.


SEQUENCE LISTING (49 sequences)

SEQ ID NO: 1 - protein, 439 aa, synthetic construct
QVQLVESGGGVVQPGRSLRLSCAASGFTFSSFGMHWVRQAPGKGLEWVAYISSASSTIYYADTVKGRFTISRDNSKNTLYLQMNSLRAEDTAVYYCASSYGYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 219 aa, synthetic construct
DIVMTQTPLSLPVTPGEPASISCRSSRSLVHSNGNTYLHWYLQKPGQSPQLLIYKVSNRFSGVPDRFSGSGSGTDFTLKISRVEAEDVGVYYCSQTTHVPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSSFGMH

SEQ ID NO: 4 - protein, 17 aa, synthetic construct
YISSASSTIYYADTVKG

SEQ ID NO: 5 - protein, 4 aa, synthetic construct
SYGY

SEQ ID NO: 6 - protein, 16 aa, synthetic construct
RSSRSLVHSNGNTYLH

SEQ ID NO: 7 - protein, 7 aa, synthetic construct
KVSNRFS

SEQ ID NO: 8 - protein, 9 aa, synthetic construct
SQTTHVPLT

SEQ ID NO: 9 - protein, 5 aa, synthetic construct
SFGMH

SEQ ID NO: 10 - protein, 17 aa, synthetic construct
YISSASSTIYYADTVKG

SEQ ID NO: 11 - protein, 4 aa, synthetic construct
SYGY

SEQ ID NO: 12 - protein, 16 aa, synthetic construct
RSSRSLVHSNGNTYLH

SEQ ID NO: 13 - protein, 7 aa, synthetic construct
KVSNRFS

SEQ ID NO: 14 - protein, 9 aa, synthetic construct
SQTTHVPLT

SEQ ID NO: 15 - protein, 8 aa, synthetic construct
GFTFSSFG

SEQ ID NO: 16 - protein, 8 aa, synthetic construct
ISSASSTI

SEQ ID NO: 17 - protein, 6 aa, synthetic construct
ASSYGY

SEQ ID NO: 18 - protein, 11 aa, synthetic construct
RSLVHSNGNTY

SEQ ID NO: 19 - skipped

SEQ ID NO: 20 - protein, 9 aa, synthetic construct
SQTTHVPLT

SEQ ID NO: 21 - protein, 113 aa, Mus musculus
DVQLVESGGGLVQPGGSRKLSCAASGFTFSSFGMHWVRQAPEKGLEWVAYISSASSTIYYADTVKGRFTISRDNPKNTLFLQMTSLRSEDTAMYYCASSYGYWGQGTTLTVSS

SEQ ID NO: 22 - protein, 113 aa, Mus musculus
DVVMTQTPLSLPVSLGDQASISCRSSRSLVHSNGNTYLHWYLQKPGQSPQLLIYKVSNRFSGVPDRFSGSGSGTDFTLKISRVEAEDLGVYFCSQTTHVPLTFGAGTRLELKR

SEQ ID NO: 23 - protein, 113 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKYFDYWGQGTLVTVSS

SEQ ID NO: 24 - protein, 114 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPITFGQGTRLEIKR

SEQ ID NO: 25 - protein, 113 aa, synthetic construct
QVQLVESGGGVVQPGRSLRLSCAASGFTFSSFGMHWVRQAPGKGLEWVAYISSASSTIYYADTVKGRFTISRDNSKNTLYLQMNSLRAEDTAVYYCAKSYGYWGQGTLVTVSS

SEQ ID NO: 26 - protein, 113 aa, synthetic construct
QVQLVESGGGVVQPGRSLRLSCAASGFTFSSFGMHWVRQAPGKGLEWVAYISSASSTIYYADTVKGRFTISRDNSKNTLYLQMNSLRAEDTAVYYCASSYGYWGQGTLVTVSS

SEQ ID NO: 27 - protein, 113 aa, synthetic construct
QVQLVESGGGVVQPGRSLRLSCAASGFTFSSFGMHWVRQAPGKGLEWVAYISSASSTIYYADTVKGRFTISRDNPKNTLYLQMNSLRAEDTAVYYCASSYGYWGQGTLVTVSS

SEQ ID NO: 28 - protein, 113 aa, synthetic construct
QVQLVESGGGVVQPGGSRRLSCAASGFTFSSFGMHWVRQAPEKGLEWVAYISSASSTIYYADTVKGRFTISRDNPKNTLYLQMNSLRAEDTAVYYCASSYGYWGQGTLVTVSS

SEQ ID NO: 29 - protein, 113 aa, synthetic construct
DIVMTQTPLSLPVTPGEPASISCRSSRSLVHSNGNTYLHWYLQKPGQSPQLLIYKVSNRFSGVPDRFSGSGSGTDFTLKISRVEAEDVGVYYCSQTTHVPLTFGQGTRLEIKR

SEQ ID NO: 30 - protein, 113 aa, synthetic construct
DIVMTQTPLSLPVTLGEQASISCRSSRSLVHSNGNTYLHWYLQKPGQSPQLLIYKVSNRFSGVPDRFSGSGSGTDFTLKISRVEAEDVGVYYCSQTTHVPLTFGQGTRLEIKR

SEQ ID NO: 31 - protein, 113 aa, synthetic construct
DIVMTQTPLSLPVTLGEQASISCRSSRSLVHSNGNTYLHWYLQKPGQSPQLLIYKVSNRFSGVPDRFSGSGSGTDFTLKISRVEAEDVGVYFCSQTTHVPLTFGAGTRLEIKR

SEQ ID NO: 32 - protein, 25 aa, Homo sapiens
QVQLVESGGGVVQPGRSLRLSCAAS

SEQ ID NO: 33 - protein, 14 aa, Homo sapiens
WVRQAPGKGLEWVA

SEQ ID NO: 34 - protein, 32 aa, Homo sapiens
RFTISRDNSKNTLYLQMNSLRAEDTAVYYCAS

SEQ ID NO: 35 - protein, 11 aa, Homo sapiens
WGQGTLVTVSS

SEQ ID NO: 36 - protein, 23 aa, Homo sapiens
DIVMTQTPLSLPVTPGEPASISC

SEQ ID NO: 37 - protein, 15 aa, Homo sapiens
WYLQKPGQSPQLLIY

SEQ ID NO: 38 - protein, 32 aa, Homo sapiens
GVPDRFSGSGSGTDFTLKISRVEAEDVGVYYC

SEQ ID NO: 39 - protein, 11 aa, Homo sapiens
FGQGTRLEIKR

SEQ ID NO: 40 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 1377 bp, synthetic construct
  source 1..1377: mol_type = other DNA
atgggctggtcctgcatcatcctgtttctggtcgctaccgccaccggcgtgcactcccaagtacaactggtcgaatctggcggcggagtcgtacaacctggccggtccctgagactgtcttgcgccgcctccggcttcaccttctcgagcttcggcatgcactgggtccgacaagcccctggcaagggcctggagtgggtcgcctacatctcctccgcctcgagcaccatctactacgccgacaccgtgaaggggagattcaccatctcccgggacaactccaaaaacaccctgtacctgcaaatgaactccctgcgggccgaggacaccgccgtgtactactgcgcctcctcctacggctactggggccaaggcaccctcgttactgtctcctctgcctccaccaagggcccctccgtgttccctctggccccttgctcccggtccacctccgagtctaccgccgctctgggctgcctggtcaaggactacttccccgagcccgtgaccgtgtcctggaactctggcgcccttacgagcggcgtgcacaccttccctgccgtgctgcaatcctccggcctgtactccctgtcctccgtcgtcaccgtgacctcgagcaacttcggcacccaaacctacacctgtaacgtggaccacaaaccctccaacaccaaggtcgacaaaaccgtggaacggaagtgctgcgtggagtgccccccctgccctgctcctcctgtggctgggccgagcgtgttcctgttccccccaaaacccaaggacaccctgatgatctcccggacccccgaggtcacctgcgtcgtcgtggacgtgtcccacgaggaccccgaggtccaattcaattggtacgtggacggcatggaggtccacaacgccaaaaccaaaccgagagaggaacaattcaactccaccttccgcgtcgtgtctgtgcttaccgtcgtgcaccaagactggctgaacggcaaagaatacaagtgcgctgtctccaacaagggcctgcctgcccccatcgaaaaaaccatctccaaaaccaagggccaaccccgcgagccccaagtctacacactgcccccgagccgggaggagatgaccaaaaaccaagtctcccttacctgtctcgtgaaaggcttctacccctccgatatcgccgtggagtgggagtccaacggccaacccgagaacaactacaaaaccaccccccccatgctggactccgacggctcattcttcctgtactccaaacttaccgtcgacaaatcccgctggcaacaaggcaacgtgttctcctgctccgtgatgcacgaggccctgcacaaccactacacccaaaaatccctgtccctgagccccggcaagtga

SEQ ID NO: 43 - DNA, 717 bp, synthetic construct
  source 1..717: mol_type = other DNA
atgggctggtcctgcatcatcctgtttctggtggctaccgccaccggcgtgcactccgacatcgtgatgacccagacccccctgtccctgcccgtgacacctggcgagcctgcctccatctcctgccggtcctcccggtccctggtgcactccaacggcaacacctacctgcactggtatctgcagaagcccggccagtccccccagctgctgatctacaaggtgtccaaccggttctccggcgtgcccgacagattctccggctccggctctggcaccgacttcaccctgaagatctcccgggtggaagccgaggacgtgggcgtgtactactgctcccagaccacccacgtgcccctgaccttcggccagggcacccggctggaaatcaagcggaccgtggccgctccctccgtgttcatcttcccaccctccgacgagcagctgaagtccggcaccgcctccgtggtctgcctgctgaacaacttctacccccgcgaggccaaggtgcagtggaaggtggacaacgccctgcagtccggcaactcccaagaatccgtgaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgctga

SEQ ID NO: 44 - protein, 10 aa, synthetic construct
GFTFSSFGMH

SEQ ID NO: 45 - protein, 6 aa, synthetic construct
SSASST

SEQ ID NO: 46 - protein, 4 aa, synthetic construct
SYGY

SEQ ID NO: 47 - protein, 12 aa, synthetic construct
SRSLVHSNGNTY

SEQ ID NO: 48 - skipped

SEQ ID NO: 49 - protein, 6 aa, synthetic construct
TTHVPL